(19) United States
(12) Patent Application Publication
(10) Pub. No.: US 2010/0144543 A1
(43) Pub. Date: Jun. 10, 2010

(54) Epigenetic silencing of tumor suppressor genes

(75) Inventors: Michael Witcher, San Diego, CA (US); Beverly M. Emerson, San Diego, CA (US)

Correspondence Address: QUINE INTELLECTUAL PROPERTY LAW GROUP, P.C., P O BOX 458, ALAMEDA, CA, 94501, US

(73) Assignee: The Salk Institute for Biological Studies, La Jolla, CA (US)

(21) Appl. No.: 12/387,455

(22) Filed: Apr. 30, 2009

Related U.S. Application Data
(60) Provisional application No. 61/126,236, filed on May 1, 2008.

(51) Int. Cl. C40B 30/04; C12Q 1/68; C12N 5/10

(52) U.S. Cl. 506/9; 435/7.1; 435/6; 435/325

(57) ABSTRACT

Provided are methods of identifying a compound that binds to or modulates an activity of a CTCF polypeptide or CTCF polypeptide complex. Also provided are methods of monitoring a cancer state of a cell by detecting a chromatin boundary proximal to a tumor suppressor gene of the cell and by monitoring the formation of a gene-specific CTCF polypeptide complex in the cell. In addition, methods of selecting a treatment or determining a prognosis for a cancer related disease are provided. Provided are recombinant cells comprising recombinant CTCF genes, recombinant cells comprising CTCF knock downs or knock outs, and recombinant laboratory animals comprising CTCF knock downs or knock outs.

### CROSS-REFERENCE TO RELATED APPLICATION

This application claims priority to and benefit of United States Provisional

Patent Application Ser. No. 61/126,236, filed May 1, 2008, the contents of which are hereby incorporated by reference in their entirety for all purposes.

### FIELD OF THE INVENTION

This invention is in the field of epigenetic regulation and cancer biology.

### BACKGROUND OF THE INVENTION

Genomic instability leading to deregulated gene expression is characteristic of human cancers and age-related diseases, e.g., Alzheimer's disease, Parkinson's disease, diabetes mellitus, and others. For example, aberrant transcriptional silencing of tumor suppressor genes by epigenetic deregulation is a common occurrence in human malignancies. This is characterized by altered patterns of DNA hypermethylation in specific promoter regions and acquisition of histone modifications that are characteristic of repressed chromatin, such as deacetylation of histones 3 and 4 and methylation of specific lysine residues like H3K9 and H3K27 (Feinberg et al. (2006) “The epigenetic progenitor origin of human cancer.” Nat Rev Genet. 7: 21-33; Feinberg (2008) “Epigenetics at the epicenter of modern medicine.” JAMA 299: 1345-1350; Jenuwein (2006) “The epigenetic magic of histone lysine methylation.” FEBS J 273: 3121-3135; Jones and Baylin (2007) “The epigenomics of cancer.” Cell 128: 683-692). Transcriptional inactivation and chromatin repression usually precede DNA hypermethylation at silenced promoters (Bachman et al. (2003). “Histone modifications and silencing prior to DNA methylation of a tumor suppressor gene.” Cancer Cell 3: 89-95; Strunnikova et al. (2005) “Chromatin inactivation precedes de novo DNA methylation during the progressive epigenetic silencing of the RASSF1A promoter.” Mol Cell Biol 25: 3923-3933). Several seminal studies have revealed insights into the interplay between methyl-DNA binding proteins, such as MeCP2, and complexes with histone deacetylase or methyltransferase activities indicating that these proteins act in concert to form repressive chromatin structures through targeted recruitment to specific promoters (Harikrishnan et al. (2005) “Brahma links the SWI/SNF chromatin-remodeling complex with MeCP2-dependent transcriptional silencing.” Nat Genet. 37: 254-264; Jones et al. (1998) “Methylated DNA and MeCP2 recruit histone deacetylase to repress transcription.” Nat Genet. 19: 187-191; Nan et al. (1998) “Transcriptional repression by the methyl-CpG-binding protein MeCP2 involves a histone deacetylase complex.” Nature 393: 386-389; Zhang et al. (1999) “Analysis of the NuRD subunits reveals a histone deacetylase core complex and a connection with DNA methylation.” Genes Dev 13: 1924-1935).

Because of its importance in cell proliferation, the human INK4 gene locus is a frequent target of inactivation by deletion or aberrant DNA methylation in a wide variety of human cancers (Kim and Sharpless (2006) “The regulation of INK4/ARF in cancer and aging.” Cell 127: 265-275; Lowe and Sherr (2003) “Tumor suppression by Ink4a-Arf: progress and puzzles.” Curr Opin Genet Dev 13: 77-83). This locus encompasses approximately 42 kb on chromosome 9 and encodes three distinct tumor suppressor proteins, p15^{INK4b}, p14^{ARF} and p16^{INK4a} (referred to hereafter as p15, p14 and p16). p16 is a key regulator of G1 phase cell cycle arrest and senescence, which it achieves primarily through inhibiting the cyclin-dependent kinases CDK4 and CDK6. Inactivation of these CDKs maintains Rb in a hypophosphorylated form enabling it to repress genes required for transition to S phase. In fact, inactivation of the p16 gene by promoter methylation or genetic change is one of the earliest losses of tumor suppressor function in numerous types of human cancers, such as breast, lung, colorectal cancers and multiple myeloma (Belinsky et al. (1998) “Aberrant methylation of p16(INK4a) is an early event in lung cancer and a potential biomarker for early diagnosis.” Proc Natl Acad Sci USA 95: 11891-11896; Esteller et al. (2001) “A gene hypermethylation profile of human cancer.” Cancer Res 61: 3225-3229; Foster et al. (1998) “Inactivation of p16 in human mammary epithelial cells by CpG island methylation.” Mol Cell Biol 18: 1793-1801; Ng et al. (1997) “Frequent hypermethylation of p16 and p15 genes in multiple myeloma.” Blood 89: 2500-2506). Notably, p16 promoter methylation and transcriptional silencing have been shown to exist in histologically normal mammary tissue of cancer-free women (FIG. 10, arrows point to some of the stained areas in the tissue). This suggests that these aberrant epigenetic changes may represent a cancerous pre-condition and an early event in promoting genomic instability that leads to tumorigenesis (Hoist et al. (2003) “Methylation of p16(INK4a) promoters occurs in vivo in histologically normal human mammary epithelia.” Cancer Res 63: 1596-1601) and the onset of aging-related diseases.

Although the precise mechanisms underlying epigenetic loss-of-function of the p16 gene remain unresolved, an examination of proteins important for its regulation may provide insight into the cause of aberrant silencing. The transcription factors Ets, JunB and Sp1 have each been shown to directly activate p16 expression through cis elements (Ohtani et al. (2001) “Opposing effects of ETS and ID proteins on p16INK4a expression during cellular senescence.” Nature 409: 1067-1070; Passegue and Wagner (2000) “JunB suppresses cell proliferation by transcriptional activation of p16(INK4a) expression.” EMBO J. 19: 2969-2979; Wu et al. (2007) “Sp1 is essential for p16 expression in human diploid fibroblasts during senescence.” PLoS ONE 2: e164). Therefore, dysfunction or aberrant recruitment of repressor complexes by these activators could result in p16 gene inactivation. However, at present little evidence exists that these factors are deregulated at the p16 promoter in cancer cells. The p38 signaling cascade has also been demonstrated to regulate p16 expression, albeit through an unknown mechanism (Bulavin et al. (2004) “Inactivation of the Wip1 phosphatase inhibits mammary tumorigenesis through p38 MAPK-mediated activation of the p16(Ink4a)-p19(Arf) pathway.” Nat Genet 36: 343-350). Constitutive activation of p38 in mice null for the phosphatase Wip-1 significantly reduces tumor formation in mouse models of breast cancer. Furthermore, this reduction is abrogated by deletion of the p16 and p14 genes. Thus, p38 signaling serves as a potential therapeutic target whereby p16 could be reactivated by stimulation of this pathway. Recently, a captivating study revealed that transcription of all three INK4/ARF genes is controlled by a common Cdc6-binding regulatory element (Gonzalez et al. (2006) “Oncogenic activity of Cdc6 through repression of the INK4/ARF locus.” Nature 440: 702-706). When this putative origin-of-replication is heterochromatinized by targeted RNA-interference, transcriptional repression of the three tumor suppressor genes ensues. Moreover, the oncogenic activity of Cdc6 is greatly reduced in INK4/ARF^{-/-} MEFs and a reciprocal abundance of p16 and Cdc6 proteins is found in non-small cell lung carcinomas. While this study is decidedly intriguing, inactivation of the INK4/ARF locus through one governing element is unlikely to be the sole cause of aberrant silencing of these tumor suppressors. This is supported by RNA expression and DNA methylation profiles of p15, p14 and p16 genes in a variety of tumors and cancer cell lines that show no obvious coupling of p16 silencing with the two other genes (Bisogna et al. (2001) “Molecular analysis of the INK4A and INK4B gene loci in human breast cancer cell lines and primary carcinomas.” Cancer Genet Cytogenet 15: 131-138; Paz et al. (2003) “A systematic profile of DNA methylation in human cancer cell lines.” Cancer Res 63: 1114-1121).

In addition to possible dysfunctional activation, p16 silencing could also result from gain-of-function or aberrant targeting of repressor proteins that modulate epigenetic processes. In this regard, several known repressors of the p16 gene may be involved. First, the ID family member IDI plays a critical role in p16 regulation during senescence in human fibroblasts through exchange of ID for ETS activators (Ohtani et al. (2001) “Opposing effects of ETS and ID proteins on p16INK4a expression during cellular senescence.” Nature 409: 1067-1070). However, it is unclear if ID-mediated repression contributes to p16 deregulation during tumorigenesis. Another repressor of p16, the polycomb group member BMI1, has been shown to have oncogenic activity (Haupt et al. (1991) “Novel zinc finger gene implicated as myc collaborator by retrovirally accelerated lymphomagenesis in E mu-myc transgenic mice.” Cell 65: 753-763; van Lohuizen et al. (1991) “Identification of cooperating oncogenes in E mu-myc transgenic mice by provirus tagging.” Cell 65: 737-752) and to control cell proliferation and senescence through the INK4a locus (Jacobs et al. (1999) “The oncogene and Polycomb-group gene BMI1 regulates cell proliferation and senescence through the ink4a locus.” Nature 397: 164-168; Smith et al. (2003) “BMI1 regulation of INK4A-ARF is a downstream requirement for transformation of hematopoietic progenitors by E2a-Pbx1.” Mol Cell 12: 393-400). BMI1 directly interacts with the p16 gene and maintains low levels of its expression in early passage proliferating fibroblasts while in senescent cells BMI1 association is lost. In primary breast tumors, however, no correlation between BMI1 and p16 expression is observed (Silva et al. (2006) “Implication of polycomb members BMI1, MeI-18, and Hpc-2 in the regulation of p16INK4a, p14ARF, h-TERT, and c-Myc expression in primary breast carcinomas.” Clin Cancer Res 12: 6929-6936). In addition to BMI1, other polycomb members such as EZH2 and Suz12 also interact with p16 in proliferating fibroblasts (Bracken et al. (2007) “The Polycomb group proteins bind throughout the INK4A-ARF locus and are disassociated in senescent cells.” Genes Dev 21: 525-530; Kotake et al. (2007) “pRB family proteins are required for H3K27 trimethylation and Polycomb repression complexes binding to and silencing p16INK4alpha tumor suppressor gene.” Genes Dev 21:49-54). Recent evidence indicates that EZH2 can recruit DNA methyltransferases to target promoters and maintain methylation patterns at silenced genes in cancer cells (Vire et al. (2006) “The Polycomb group protein EZH2 directly controls DNA methylation.” Nature 439: 871-874). As EZH2 was shown to bind the p16 gene in fibroblasts, deregulation of EZH2 may represent a direct link to epigenetic changes occurring at the p16 locus during oncogenesis.

Further understanding of the epigenetic mechanisms of gene silencing would be useful, e.g., to provide methods and compositions for assessing the cancer state of a cell, thereby providing diagnostic and prognostic tools for the clinician. In addition, if the epigenetic mechanisms were better understood, it would provide new therapeutic drug targets, and would allow for the identification of modulators of these targets. The present invention provides these and other features that will become apparent upon complete review.

### SUMMARY OF THE INVENTION

The deregulation of epigenetic modifications can contribute to the pathogenesis of many cancers and other gene regulation disorders, e.g., age-related diseases such as Alzheimer's disease, Parkinson's disease, cardiovascular disease, diabetes mellitus, and others. One of the epigenetic characteristics that can be altered in malignant cells is the maintenance of higher-order chromosomal domains through appropriate chromosomal boundary formation, e.g., by the binding of CTCF insulator elements. When the integrity of a chromosomal boundary is compromised, e.g., by destabilized CTCF binding, the loss of long-range epigenetic organization can be accompanied by the silencing of tumor suppressor genes. Thus, the spread of repressive heterochromatin and DNA hypermethylation from a transcriptionally inactive domain into a neighboring region of active genes can result in aberrant gene silencing, commonly found in human cancers and aging cells. The invention is generally directed to methods and compositions that can be used to identify compounds that modulate the stability of chromosomal boundaries. The invention also provides methods that can be used to detect the destabilization of chromatin boundaries as a means to monitor the disease state of the cell, to select a treatment, and/or to determine the prognosis of, e.g., a cancer-related disease.

In a first aspect, the invention provides methods of identifying a compound that binds to or modulates an activity of a CTCF polypeptide or CTCF complex. The methods include contacting a biological or biochemical sample comprising the CTCF polypeptide or complex with a test compound and detecting either binding of the test compound to the CTCF polypeptide or complex or modulation of the activity of the CTCF polypeptide or complex by the test compound. The modulator, e.g., test compound that affects the activity of a CTCF polypeptide or complex, can optionally induce, or potentiate, the activity of the polypeptide or complex, e.g., by promoting the poly(ADP-ribosyl)ation of CTCF or a CTCF-associated cofactor, or it can inhibit the activity of the polypeptide or complex, e.g., by preventing the poly(ADP-ribosyl)ation of CTCF or CTCF-associated cofactor.

The biological or biochemical sample that includes the CTCF polypeptide or CTCF complex can optionally comprise a cancer cell, a multiple myeloma cell, a U266 cell, a KMS12 cell, a breast cancer cell, a T4D7 cell, a primary breast epithelial cancer cell, a vHMEC cell, a cervical cancer cell, a normal human mammary epithelial cell (HMEC), a HeLa cell, a non-transformed fibroblast cell, an MDA-MB-435 cell, an LMR90 cell, a primary cancer cell from a patient, or, e.g., a cell derived through culture from a primary cancer cell from a patient. In a particular embodiment, the sample that comprises the CTCF polypeptide or complex can comprise a tumor suppressor gene, and the activity of the CTCF polypeptide or complex that is modulated by the test compound can optionally comprise suppression of the tumor suppressor gene, e.g., gene silencing of the tumor suppressor gene, or restoration of tumor suppressor gene expression.

The test compound screened in the methods can optionally modulate any of a number of activities of the CTCF polypeptide or CTCF complex in a biological or biochemical sample. The modulated activity can optionally include the induction or loss of tumorigenesis in a cell present in the biological or biochemical sample or the binding of CTCF to a histone, a post-translationally modified histone, a chromatin, or a chromatin boundary in the biological or biochemical sample. The modulated activity can optionally include chromatin boundary stabilization, insulation or formation, or suppression of a loss of a chromosome boundary during gene silencing in the biological or biochemical sample that comprises the CTCF polypeptide or CTCF polypeptide complex.

The activity of the CTCF polypeptide or CTCF polypeptide complex that is modulated by the test compound and monitored by the methods can optionally include binding of the CTCF polypeptide or CTCF complex to a chromatin boundary within or proximal to an INK4/ARF gene locus, a p16^{INK4a} gene, a RASSF1a gene, a CDH1 gene or a C-Myc gene present in the biological or biochemical sample. Optionally, the modulated activity can include activation of a p16^{INK4a} gene, a RASSF1a gene, a CDH1 gene or a C-Myc gene present in the biological or biochemical sample. The modulated activity that is monitored in the methods can optionally include stabilization of tumor suppressor gene reactivation for a tumor suppressor gene present in the biological or biochemical sample.

The activity of the CTCF polypeptide or complex that is modulated by a test compound can optionally comprise one or more of activities that include: an increase or decrease in aberrant methylation in or proximal to a promoter or gene of interest; an increase or decrease in H2A.Z binding proximal to or within a promoter or gene of interest; an increase or decrease in trimethylation of H3K4 proximal to or within a promoter or gene of interest; an increase or decrease in monomethylation of H4K20 proximal to or within a promoter or gene of interest; an increase or decrease in dimethylation of H3K27 proximal to or within a promoter or gene of interest; or an increase or decrease in trimethylation of H3K9 proximal to or within a promoter or gene of interest.

In a particular embodiment, the activity of the CTCF polypeptide or complex that is monitored in the methods comprises the formation of an active CTCF polypeptide complex, e.g., a gene specific complex, in the biological or biochemical sample. The active complex can optionally comprise CHD8, YB-1, Topoisomerase IIα, Topoisomerase IIβ, Nucleolin, Nucleophosmin, Poly(ADP-ribose) polymerase (PARP1), Importin alpha3/alpha1, Lamin A/C, YB-1, YY1, a DNA repair enzyme, RAD50, MRE11, XRCC6/KU80, a SWI/SNF chromatin remodeling enzyme, TFII-i, and/or H2A.Z. The active complex can optionally comprise one or more post-translational modification, e.g., PARlation and/or phosphorylation. For example, where the gene is p16, the CTCF complex can include Topoisomerse IIβ.

The methods of identifying a compound that binds to or modulates the activity of a CTCF polypeptide or complex can comprise screening a plurality of test compounds, which can optionally be prescreened for bioavailability, oral availability, toxicity, and/or transport to the nucleus. The compound screened for its effects on the activity of a CTCF polypeptide or complex can optionally be a kinase inhibitor, a phosphatase inhibitor, a post-translational modification reagent, a nucleoside analogue, a nucleotide analogue, a methylation reagent, a hypomethylating nucleoside analogue, an HDAC inhibitor, a polypeptide, a naturally occurring compound, a small organic molecule, or the like.

Any of the compounds screened by the methods can optionally be members of a combinatorial compound library. The combinatorial compound library that is screened by the methods can optionally be selected or pre-selected for any features or properties of interest, as noted above. For example, the compounds can be selected to comprise a majority of members that conform to Lipinski's rule of 5, e.g., by providing that each member of the majority comprise not more than 5 hydrogen bond donors, not more than 10 hydrogen bond acceptors, a molecular weight under 500 g/mol and a partition coefficient log P less than 5. The combinatorial compound library screened by the methods can optionally be based upon, e.g., at least one pharmacophore scaffold. Optionally, the combinatorial compound library can be based upon up to about 45 different pharmacophore scaffolds, where each scaffold is represented in the library by a plurality of members, and the overall library comprises at least about 4,000 unique compounds. Each scaffold can optionally represent, on average, about 96 members.

The invention also provides methods of monitoring a cancer or age-related disease state of a cell, which include detecting destabilization of a chromatin boundary proximal to a gene of the cell, e.g., a tumor suppressor gene, wherein destabilization of the chromatin boundary correlates with genomic instability or a tumorigenesis process in the cell. The methods can optionally be performed on cells from a cancer cell culture, primary cells from a patient, or cells that are derived from primary cells from a patient. The methods can optionally be used with, e.g., a cell from a cancer cell culture, a primary cell from a patient, or a cell that is derived from a primary cell from a patient. Optionally, the methods can be used with any of the cells or cells lines described previously.

In the methods of monitoring a cancer or other disease state of a cell, the destabilization of the chromatin boundary can optionally be detected by detecting binding of a CTCF protein or protein complex to the chromatin boundary, e.g., a chromatin boundary within or proximal to an INK4/ARF gene locus, a p16^{INK4a} gene, a RASSF1a gene, a CDH1 gene, or a C-Myc gene. In a particular embodiment, the destabilization of the chromatin boundary is measured by performing a chromatin immunoprecipitation assay using an antibody specific for a CTCF protein, thereby identifying chromatin regions bound by the CTCF protein. Destabilization of the chromatin boundary can also be detected by detecting an un poly(ADP-ribosyl)ated CTCF protein or by detecting a stable CTCF/PARP-1 complex. In one exemplary embodiment, this can be measured by performing a co-immunoprecipitation using an antibody specific for CTCF protein and detecting PARlation of the CTCF protein with an antibody specific for poly(ADP-ribose) polymer. As noted above, the absence of CTCF PARlation indicates destabilization of the chromatin boundary.

In a related aspect, the invention provides methods of selecting a treatment or determining a prognosis for a cancer- or age-related disease. The methods include measuring CTCF protein or CTCF complex binding within or proximal to a gene, e.g., a tumor suppressor gene, in a patient, wherein CTCF protein or complex binding within or proximal to the gene is correlated with disease progression, or treatment selection. The methods also include providing a patient prognosis based upon the CTCF protein or complex binding, or selecting a treatment course based upon the CTCF protein or complex binding. The binding of the CTCF protein or complex can optionally be measured by performing a chromatin immunoprecipitation using an antibody specific for the CTCF protein to identify chromatin regions bound by the CTCF protein.

The binding of the CTCF protein or complex can optionally correlate with long term reestablishment of the gene's activity by an epigenetic therapeutic agent, and the lack of binding of said CTCF protein or complex can optionally correlate with failure in long term reestablishment of the gene's activity by an epigenetic therapeutic agent.

Where the CTCF protein or complex fails to bind within or proximal to the gene, the methods can optionally further include testing CTCF protein or complex binding within or proximal to a second gene, e.g., a second tumor suppressor gene, wherein the CTCF protein or complex binds to a second gene. Optionally, binding of the CTCF protein or complex within or proximal to the second gene can provide an indication that a disease cell of the patient displays CTCF activity or expression, with a defect in either gene-specific CTCF complex formation or activity, or a cis-defect in gene-specific CTCF protein or complex binding. The determination that the disease cell displays CTCF binding to the second gene can optionally provide an indication regarding which gene activity should be therapeutically targeted in the patient.

The invention provides a related method of selecting a treatment or determining a prognosis for a cancer related disease. The method includes measuring CTCF polypeptide poly(ADP-ribosyl)ation in a patient, wherein decreased or absent CTCF poly(ADP-ribosyl)ation is correlated with disease progression, or treatment selection, and providing a patient prognosis based upon said CTCF poly(ADP-ribosyl)ation or selecting a treatment course based upon said CTCF poly(ADP-ribosyl)ation. A method of selecting a treatment or determining a prognosis for a cancer- or age-related disease that is provided by the invention includes determining a protein PARlation profile of a biological sample derived from a patient, and providing a patient prognosis based upon said protein PARlation profile.

The invention provides a related method of monitoring a cancer state of a cell that comprises detecting the formation, or lack of formation, of a gene-specific CTCF polypeptide complex in the cell. For example, where the gene is p16, the CTCF complex can include a Topoisomerse IIβ.

Compositions provided by the invention include recombinant cells, e.g., cells present as cells of a recombinant non-human laboratory animal, which include a recombinant gene comprising a gene encoding CTCF under the control of a heterologous promoter and a recombinant gene comprising a tumor suppressor promoter operably linked to a reporter. The heterologous promoter can optionally comprise an inducible promoter or a constitutive promoter. The reporter can optionally be homologously recombined into a chromosome of the cell, e.g., a cell of a recombinant non-human laboratory animal, at a position corresponding to the tumor suppressor gene, preserving epigenetic programming of the tumor suppressor promoter and proximal chromosomal regions.

In addition, the invention provides recombinant cells, or recombinant laboratory animals, that comprise a CTCF gene knock down or knock out. In these cells or laboratory animals, CTCF gene expression can optionally be knocked down by expression of a recombinant antisense RNA, siRNA or shRNA against the CTCF gene in the cell or animal.

The invention provides methods of identifying members of a CTCF protein complex that include providing a cellular extract derived from a target biological sample, performing gel filtration on the extract and collecting eluted fractions, performing western analysis on the eluted fractions with a CTCF antibody to detect the CTCF protein complex in the fractions, electrophoresing the fractions comprising the CTCF protein complex on an SDS-PAGE to resolve individual protein bands, and excising the individual protein bands and performing MALDI-TOF. Another method of identifying members of a CTCF protein complex that is provided by the invention includes providing a cellular extract derived from a target biological sample, immunoprecipitating the extract with an antibody specific for CTCF to precipitate the CTCF protein complex, electrophoresing the CTCF protein complex on an SDS-PAGE to resolve individual protein bands, and excising the individual protein bands and performing MALDI-TOF, thereby identifying members of the CTCF protein complex.

The invention provides methods useful for determining a protein PARlation profile of a biological sample. The methods include providing a cellular extract derived from the biological sample, incubating the extract with a microarray comprising a plurality of full-length recombinant proteins in the presence of fluorescent β-NAD⁺, and washing and scanning the microarray with a fluorescent microarray scanner to measure incorporation of β-NAD⁺, thereby determining the protein PARlation profile.

Those of skill in the art will appreciate that that the methods and compositions provided as described herein can be used alone or in combination to, e.g., for identifying compounds that bind or modulate an activity of a CTCF protein or complex, monitor the disease state of a cell, or to develop therapeutic agents to treat disease states that result from, e.g., epigenetic deregulation. Modulators of an activity of a CTCF protein or complex that are identified using the methods described herein are likewise a feature of the invention.

Library screening systems that include any of the methods or compositions described herein are also a feature of the invention. In addition to modulator libraries, and CTCF related reagents, such systems can optionally additionally include data processing and control software (e.g., for automated detection of CTCF activity or binding), liquid handling devices (e.g., for flowing CTCF and modulator reagents), detectors (for detecting on or more CTCF activity or binding event, e.g., in the presence of a modulator), and/or the like.

Kits that permit a practitioner to use the methods described herein, e.g., to monitor the cancer state of a cell, or to select a treatment and/or determine a prognosis for a cancer-related disease in a subject are also a feature of this invention. The kits can include modulators of an activity of a CTCF protein or complex, recombinant CTCF, recombinant constructs comprising genes encoding, e.g., CTCF or other complex components, modulators of CTCF protein or complex activity, an INK4/ARF locus, a p16^{INK4a} gene or gene region, a RASSF1A gene or gene region, a CDH1 gene or gene region, a c-Myc gene or gene region, and/or the like. The kits can also include additional useful reagents, such as antibodies, buffers, and the like. Such kits also typically include, e.g., instructions for use of the compounds and other reagents, e.g., to practice the methods of the invention, as well as any packaging materials for packaging the components of the kits.

### BRIEF DESCRIPTION OF THE FIGURES

FIG. 1 provides a diagram of gene organization at the INK4/ARF chromosomal locus and depicts the results of experiments performed to analyze histone modifications at the p16 gene.

FIG. 2 depicts the results of experiments performed to show that CTCF associates with the active p16 gene but not silent p16 gene.

FIG. 3 provides the results of experiments that were performed to determine whether CTCF knockdown results in transcriptional silencing of the p16 gene and/or in the acquisition of repressive chromatin modifications.

depicts the results of experiments performed to show that CTCF binding correlates with p16 expression in multiple types of human cancer cells.

FIG. 4 provides the results of experiments that were performed to determine whether CTCF is differentially Poly(ADP-ribosyl)ated in p16-expressing and p16 non-expressing breast cancer cells.

FIG. 5 depicts the results of experiments performed to determine the pattern of PARlation at the p16 promoter region changes in p16-silenced cells.

FIG. 6 depicts the results of experiments performed to show that CTCF binding is lost at loci at or near genes that are commonly silenced in human cancers.

FIG. 7 provides a model that illustrates the role of CTCF in aberrant tumor suppressor gene silencing in human cancers.

FIG. 8 provides a diagram that shows the frequency with which the promoters of certain tumor suppressor genes are silenced in tumors derived from different tissues.

FIG. 9 provides a diagram of gene organization at the INK4/ARF chromosomal locus and a schematic of how p16 inhibits the transition into S phase.

FIG. 10 depicts a region of histologically normal mammary epithelia in which p16 promoter hypermethylation is detected and adjacent stromal fibroblasts in which p16 promoter hypermethylation is not detected.

FIG. 11 provides a model of a CpG island-containing promoter in an active or silenced state.

FIG. 12 provides a schematic of the p16/Ink4a promoter and the putative response elements that participate in is transcriptional activation and repression.

FIG. 13 provides a table of various CTCF interaction partners.

FIG. 14 provides a schematic of a model of CTCF binding in the Igf2/H19 imprinting control region.

FIG. 15 provides a schematic diagram showing the results of bisulphate sequencing of the CTCF-associated region upstream of the p16 gene in p16-expressing and p16 non-expressing cells.

FIG. 16 shows the results of experiments that were performed to show that the inhibition of p16 and RASSF1A transcription does not impact CTCF binding.

FIG. 17 provides the results the analysis of BORIS expression in human cancer cells, the results of qPCR of p16 mRNA levels in CTCF knockdown cells, and qPCR of p16 mRNA levels T4D7 cells treated with AZA or trichostatin A.

FIG. 18 provides the results of experiments performed to analyze the expression and PARlation of full-length recombinant CTCF in T4D7 cells.

FIG. 19 provides a table of primer sets used in ChIP experiments described herein.

FIG. 20 provides the results of experiments performed to analyze CTCF binding and cellular localization.

FIG. 21 depicts the results of additional experiments performed to show that CTCF binding correlates with p16 expression in multiple types of human cancer cells.

FIG. 22A provides the results of ChIP analysis of CTCF binding in p16-expressing and non-expressing breast cancer cells. FIG. 22B provides a western blot of CTCF protein expression.

FIG. 23 provides a schematic model of CTCF and tumor suppressor gene silencing.

FIG. 24 provides a list CTCF interacting proteins identified via mass spectrometry.

### DETAILED DESCRIPTION

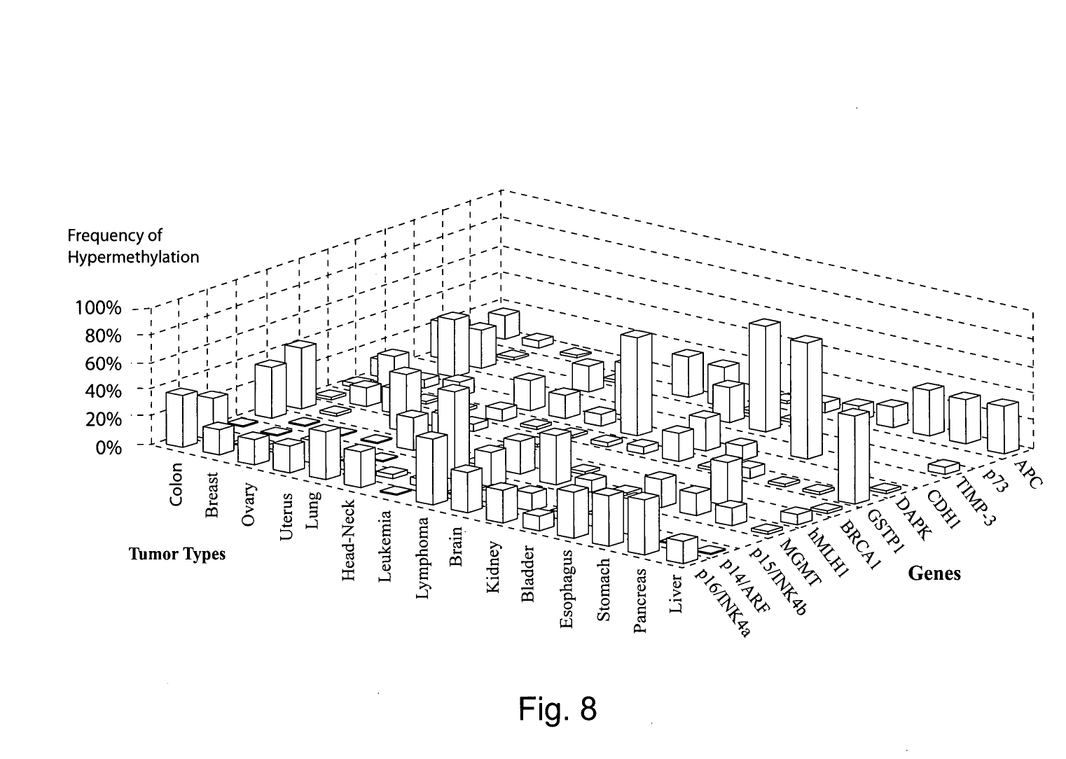

Tumor suppressor genes are inactivated in many human cancers, and, in many instances, the silencing of tumor suppressor genes is correlated with hypermethylation of the promoters from which they are transcribed (see FIG. 8). The spread of repressive chromatin and DNA hypermethylation from a transcriptionally inactive domain into a neighboring region of transcriptionally active genes can result in aberrant gene silencing that is also a hallmark of aging-related diseases such as, e.g., Alzheimer's disease, diabetes mellitus, and others. This invention describes chromatin boundaries upstream of tumor suppressor genes, such as p16^{INK4a}, that are lost when tumor suppressor genes are aberrantly silenced (e.g., resulting in a cascade of events that leads to aberrant gene activation and unregulated cell proliferation). The multifunctional protein CTCF (and/or complexes thereof) associates in the vicinity of this boundary. Loss of CTCF/complex binding and/or loss of CTCF PARlation strongly coincide with gene silencing (e.g., tumor suppressor gene silencing) in multiple cancers. A causal role for CTCF in epigenetic programming and activation of tumor suppressor genes is also demonstrated herein. Similarly, CTCF binding and/or CTCF PARlation correlates with activation of tumor suppressor genes such as RASSF1a and CDH1 genes, with these characteristics being absent when these genes are methylated and silenced. Thus, destabilization of specific chromosomal boundaries is a general mechanism to inactivate tumor suppressor genes and to initiate tumorigenesis in numerous forms of human cancers. Furthermore, treatment with a hypomethylation agent such as AZA does not automatically restore CTCF binding. This can lead to long term gene silencing, even following AZA or other hypomethylation treatment(s). Thus, CTCF binding status proximal to a tumor suppressor gene and CTCF PARlation state have a variety of diagnostic and prognostic implications. The cancer state of cells can usefully be considered with reference to binding; defects in CTCF binding that are not restored following, e.g., AZA treatment are more likely to require additional treatment, as defects in CTCF binding correlate with long term silencing. In addition, the cancer state of cells can also be monitored by determining the PARlation state of CTCF, e.g., wherein loss of CTCF PARlation correlates with long term tumor suppressor gene silencing.

This identification of CTCF in a causal role in tumor suppressor gene inactivation also provides a target for the identification of compounds that modulate an activity of CTCF or a CTCF complex. Modulators and libraries of potential modulators are formed from any of a variety of components, e.g., pharmacophore scaffolds, e.g., pre-selected for bioavailability (e.g., oral availability), or the like. Such modulators can activate or suppress gene silencing of, e.g., tumor suppressor genes, and/or can restore tumor suppressor gene expression. CTCF modulators can also be selected to stabilize tumor suppressor gene reactivation. Other modulator activities to be selected for include: an increase or decrease in aberrant methylation in or proximal to a promoter or gene of interest; an increase or decrease in histone (e.g., H2A.Z) binding proximal to or within a promoter or gene of interest; an increase or decrease in trimethylation of H3K4 proximal to or within a promoter or gene of interest; an increase or decrease in monomethylation of H4K20 proximal to or within a promoter or gene of interest; an increase or decrease in dimethylation of H3K27 proximal to or within a promoter or gene of interest; an increase or decrease in trimethylation of H3K9 proximal to or within a promoter or gene of interest, an increase or decrease in CTCF PARlation, and/or the like.

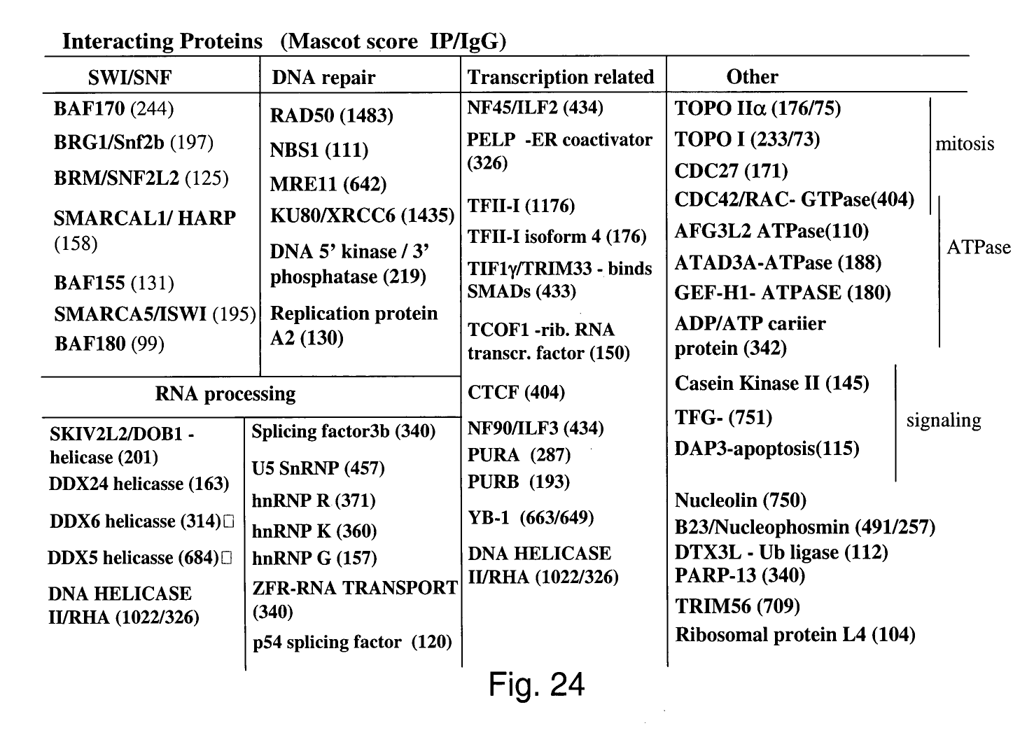

The active CTCF complex can include, e.g., CHD8, YB-1, Nucleolin, Topoisomerase IIα, Topoisomerase IIβ, Nucleophosmin, Poly(ADP-ribose) polymerase (PARP1), Importin alpha3/alpha1, Lamin A/C, YY1, a DNA repair enzyme, RAD50, MRE11, XRCC6/KU80, a SWI/SNF chromatin remodeling enzyme, TFII-i and/or H2A.Z. (See FIG. 24 for a list of CTCF interacting proteins identified via mass spectrometry.) Several of these CTCF complex components are newly described as components of CTCF complexes herein. The relevant complex (or CTCF polypeptide) can be post translationally modified, e.g., by phosphorylation or PARlation; modulators can be selected to affect or effect any such post-translational modification, e.g., as a kinase or phosphatase inhibitor or activator or a PARlation activator or inhibitor. CTCF activity/binding and PARlation state can be measured in a variety of cells, e.g., various normal and cancer cells and cell cultures as noted herein. A variety of assays for measuring CTCF activity can be performed according to the invention, including, e.g., chromatin immunoprecipitation (ChIP) assays (e.g., to measure chromatin boundary destabilization), immunoprecipitation assays using an antibody specific for Poly(ADP-ribose) polymers, expression assays to monitor expression of a gene of interest (e.g., a tumor suppressor, or a reporter localized to a relevant chromatin region) and/or the like.

Also provided are methods of selecting treatment and/or determining a prognosis for a cancer related disease. In these methods, CTCF protein or CTCF complex binding and/or CTCF PARlation is measured, e.g., within or proximal to a tumor suppressor gene in a patient. The CTCF protein or complex binding within or proximal to the tumor suppressor gene and/or CTCF PARlation state is correlated with disease progression or treatment selection, and a patient prognosis or treatment course is identified based upon the correlation. For example, binding of the CTCF protein or complex and/or CTCF PARlation correlates with long term reestablishment of tumor suppressor activity by an epigenetic therapeutic agent (e.g., an agent that re-establishes normal methylation and/or CTCF binding to a relevant chromosomal region). In contrast, lack of CTCF binding and/or loss of CTCF PARlation indicates that reestablishment of tumor suppressor expression is likely to be short term, indicating a relatively poor prognosis and/or that additional treatments can be appropriate.

Specificity of any CTCF binding defect can also be tested, e.g., to determine whether a cell (e.g., derived from a patient) displays normal CTCF binding to one or more tumor suppressor genes, while displaying abnormal binding to another. This allows for the identification of a specific epigenetic lesion(s) at issue for a patient, providing a clinician with the ability to target treatments against the epigenetic lesion(s) at issue. Abnormal versus normal CTCF binding profiles also provide an indication of whether lack of binding of the CTCF complex is a cis- or a trans-defect (or both), providing an additional indication as to underlying cause of a cancer or other gene regulatory disorder. Such increased treatment and diagnostic specificity provides improved clinical outcomes for the patient, by allowing the clinician to tailor patient treatments to match the underlying cause of a disease. Similarly, modulator screening can be used to identify modulators that target particular epigenetic lesions, cis-versus trans-defects, or the like.

Furthermore, by providing an ability to specifically profile the CTCF binding/CTCT PARlation/epigenetic defects of a cell, the invention provides a general method for monitoring the cancer state of a cell (e.g., by determining which defects appear in which cell types). Here again, this improves a clinician's ability to identify modulators and/or to specifically target the cancer cell type. This improves both the specificity of patient treatment and the specificity of any drug screening platform designed to target particular cell types (having particular cancer states).

Recombinant cells and non-human laboratory animals, useful in screening modulators for CTCF binding or activity modulation are also a feature of the invention. For example, such recombinant cells can include a recombinant gene comprising a gene encoding CTCF under the control of a heterologous promoter (e.g., an inducible or constitutive promoter, depending, e.g., on the format of the relevant assay), along with, e.g., a recombinant gene that has a tumor suppressor promoter operably linked to a reporter. Homologous recombination can also be used for the cell or animal to place the reporter gene under the control of the actual tumor suppressor promoter, e.g., to position the reporter in the same chromosomal location as the tumor suppressor, providing an easy readout of the transcriptional activity state of a chromatin region. CTCF knock down or knock out cells and animals can also be created as model systems for studying CTCF (or CTCF modulator) function.

### Activities of a CTCF Protein or CTCF Complex

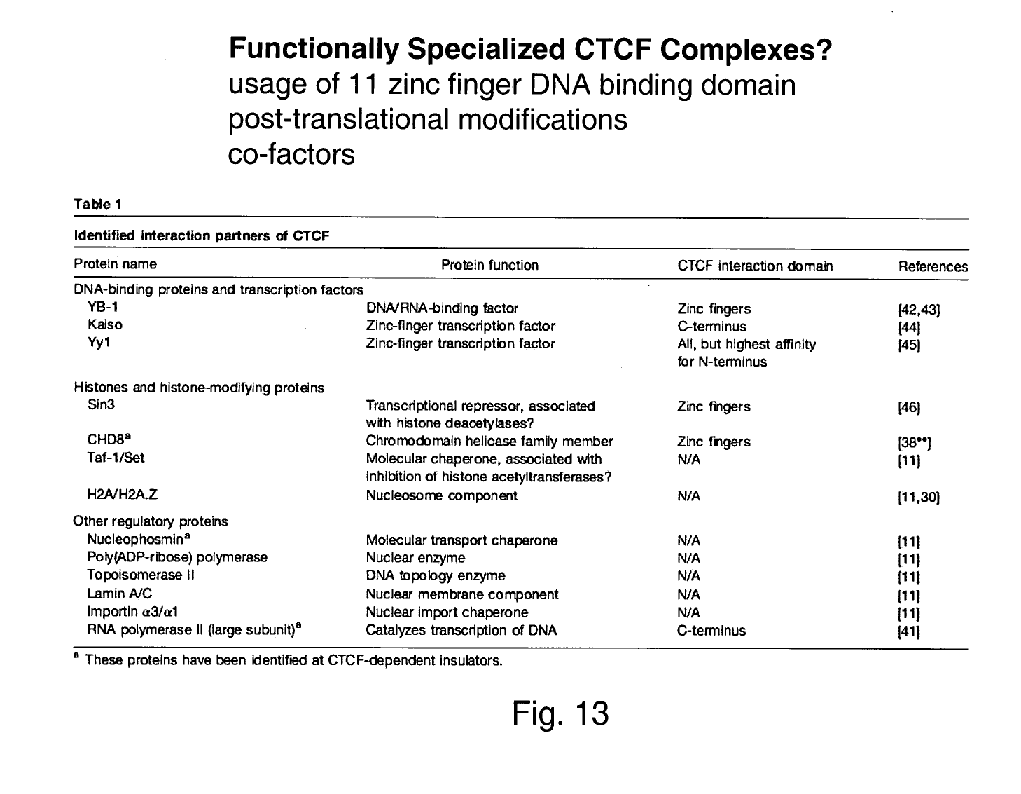

CTCF is a multifunctional zinc finger protein that plays a role in the establishment and maintenance of higher-order chromosomal domains. It also serves as either a positive or negative transcription factor on numerous target genes such as c-MYC and IGF2/H19 (Feinberg (2008) “Epigenetics at the epicenter of modern medicine.” JAMA 299: 1345-1350; Jones and Baylin (2007) “The epigenomics of cancer.” Cell 128: 683-692). CTCF associates at many genomic locations (Kim et al. (2007) “Analysis of the vertebrate insulator protein CTCF-binding sites in the human genome.” Cell 128: 1231-1245) and can form scaffolds to assemble long-range chromosomal loop Structures bringing distal genes into close proximity (Li, et al. (2008) “CTCF regulates allelic expression of Igf2 by orchestrating a promoter-polycomb repressive complex 2 intrachromosomal loop.” Mol Cell Biol 28: 6473-6482). Deregulation of CTCF leads to loss of imprinting for IGF2, a paracrine growth factor, which is found in normal aging tissues and is important for cancer progression (Vu et al. (2008)“Aging and cancer-related loss of insulin-like growth factor 2 imprinting in the mouse and human prostate.” Cancer Res. 68: 6797:6802). The diverse functions of CTCF can be imparted by its ability to be post-translationally modified by phosphorylation and poly(ADP-ribosyl)ation (PARlation) and cooperate with co-factors such as Topoisomerase H, Nucleophosmin, YY1, and PARP-1 (Wallace and Felsenfeld (2007) “We gather together: insulators and genome organization.” Curr Opin Genet Dev 17: 400-407; Filippova (2008) “Genetics and epigenetics of the multifunctional protein CTCF.” Curr Top Dev Biol 80: 337-360). The functions of a CTCF complex can likewise be affected by the post-translational modification, e.g., phosphorylation and/or PARlation, of CTCF-associated cofactors (see FIGS. 13 and 24 for lists of CTCF-associated cofactors). As described in the Example below, Nucleolin, as well as CTCF, undergoes defective PARlation in p16-silenced cells, indicating that aberrantly modified cofactors can also impact the function of an entire CTCF polypeptide complex.

One aspect of the invention is the discovery that CTCF protein and/or complex binding at chromatin boundaries and CTCF PARLation permits the long-term expression of a variety of tumor suppressor genes, e.g., p16, RASSF1a, CDH1, and c-Myc. Methods provided by the invention, e.g., methods of identifying a modulator of a CTCF protein or a CTCF complex, methods of selecting a treatment for or determining the prognosis of a cancer-related disease, and methods of monitoring the cancer state of a cell, each can entail monitoring an activity of a CTCF protein or complex.

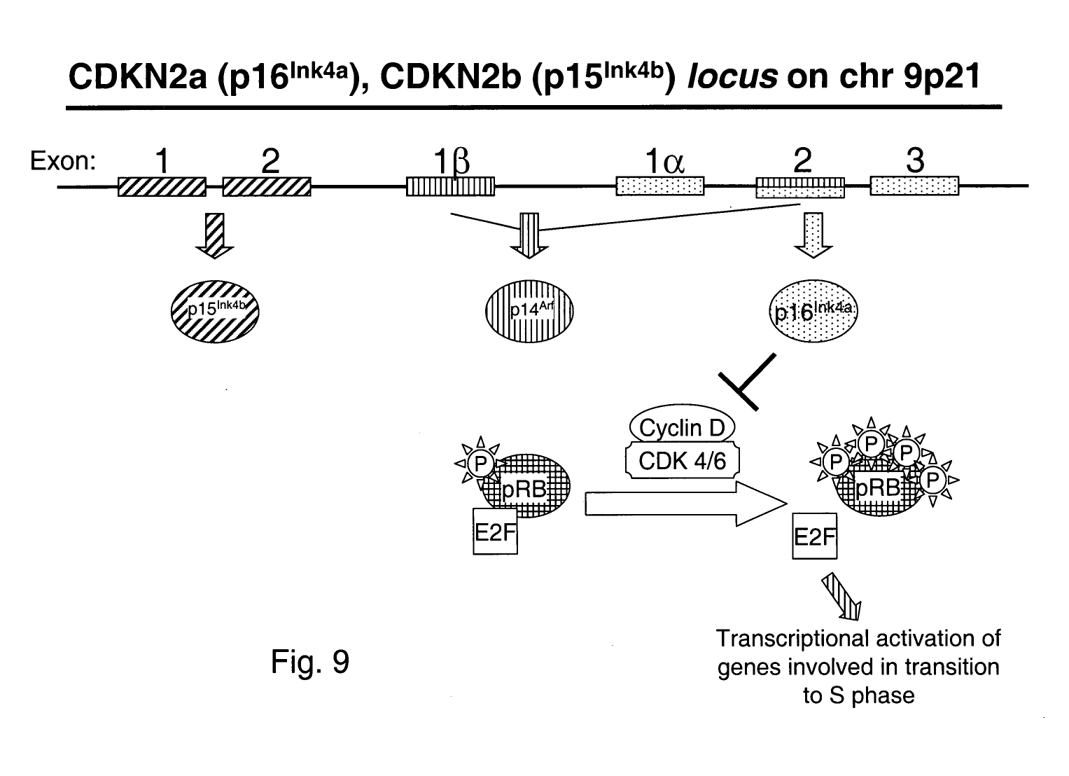

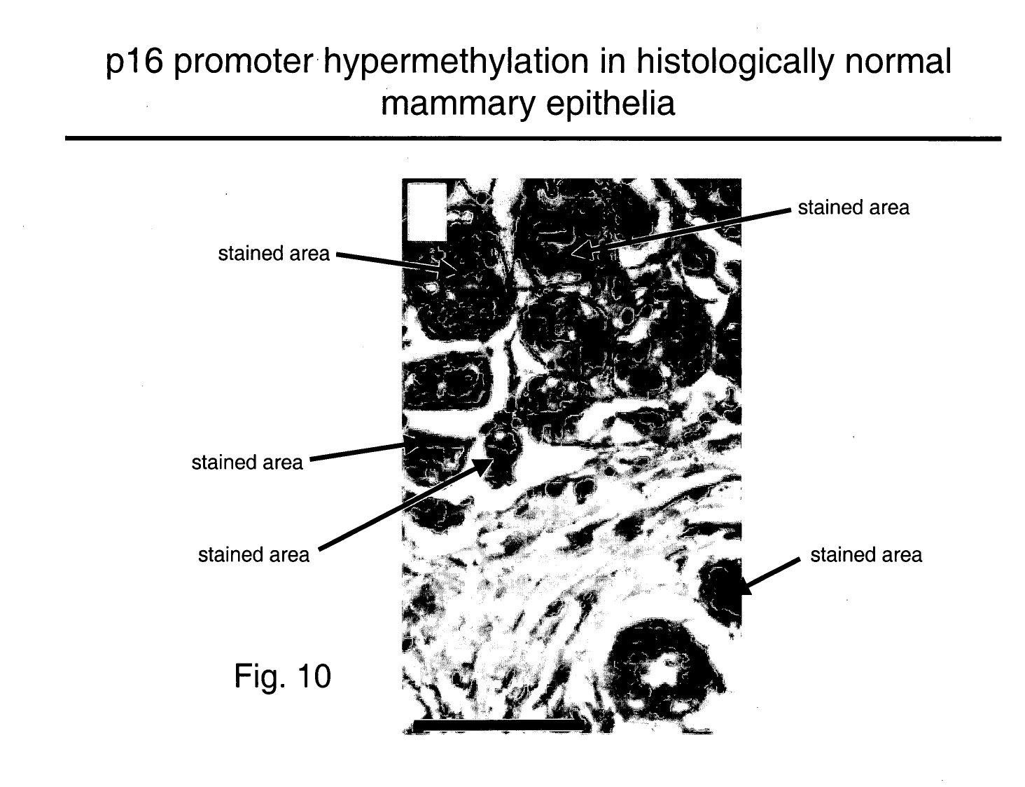

Activities of a CTCF protein or complex, e.g., that can be assayed in methods of identifying modulators of a CTCF protein or complex and/or in methods of monitoring the cancer state of a cell, include the induction or loss of tumorigenesis or tumorigenesis potential in a cell, the CTCF PARlation state, and/or the binding of a CTCF protein or complex to a histone, a post-translationally modified histone, a chromatin, or a chromatin boundary, e.g., proximal to a tumor suppressor gene present in a biological or biochemical sample. In addition, the activities of a CTCF protein or complex that can be monitored in these methods include the stabilization, insulation and/or formation of a chromatin boundary, and/or the suppression of a loss of a chromosome boundary during gene silencing or activation in a target sample. Other cellular activities influenced by a CTCF protein or complex, e.g., that can be beneficially observed in any one or more of the methods provided by the invention, are the binding of the CTCF protein or complex to a chromatin boundary proximal to or within the INK4/ARF locus (FIG. 9), the p16^{INK4a} gene, the RASSF1a gene, the CDH1 gene, the c-Myc gene and/or activation of the aforementioned genes, determining the PARlation state of a CTCF derived from a target biological sample (e.g., those described below), and/or the stabilization of tumor suppressor gene reactivation for a tumor suppressor gene present in a biological or biochemical sample comprising a CTCF protein or complex.

Insulator function and IGF2 imprinting have been shown to require post-translational modification of CTCF by PARlation (Yu et al. (2004) “Poly(ADP-ribosyl)ation regulates CTCF-dependent chromatin insulation.” Nat Genet. 36: 1105-1110) and crosstalk between PARP-1 and CTCF strongly affects DNA methylation (Guastafierro et al. (2008) “CCCTC-binding factor activates PARP-1 affecting DNA methylation machinery.” J Biol Chem 283: 21873-21880). Thus, activities of a CTCF protein or complex, e.g., that can be monitored in the methods described herein, also include an increase or decrease in aberrant methylation in or proximal to a promoter or gene of interest using e.g., a real-time PCR-based assay (described in Cottrell et al. (2004) “A real-time PCR assay for DNA-methylation using methylation-specific blockers.” Nucl Acids Res 32: e10) or LUMA (described in Karimi et al. (2006) “LUMA (Luminometric Methylation Assay)—a high throughput method to the analysis of genomic DNA methylation.” Exp Cell Res 312: 1989-1995).

A variety of histone modifications in the vicinity of, e.g., a tumor suppressor gene of interest, can be assayed to monitor the activity of a CTCF protein or complex as well. For example, an increases in H2A.Z binding and/or trimethylated H3K4 binding proximal to or within a promoter or gene of interest correlate with mammalian gene activation and can indicate an increase in CTCF protein or complex activity at a chromosomal locus. Conversely, an increase the binding of monomethylated H4K20, dimethylated H3K27, and/or trimethylated H3K9 proximal to or within a promoter or gene of interest is typically associated with repressed chromatin, and can indicated decreased CTCF activity, e.g., at the chromosomal locus of, e.g., a tumor suppressor gene of interest. The binding of H2A.Z, trimethylated H3K4, monomethylated H4K20, dimethylated H3K27, and/or trimethylated H3K9 can be assayed by chromatin immunoprecipitations (ChIP), which are descried elsewhere herein.

In a particular embodiment, the activity of the CTCF polypeptide or complex that is monitored in the methods comprises the formation of an active CTCF polypeptide complex, e.g., a gene specific complex, in the biological or biochemical sample. As described elsewhere herein, active CTCF complex can optionally comprise CHD8, Topoisomerase IIα, Topoisomerase IIβ, Nucleolin, Nucleophosmin, Poly(ADP-ribose) polymerase (PARP1), Importin alpha3/alpha1, Lamin A/C, YB-1, YY1, a DNA repair enzyme, RAD50, MRE11, XRCC6/KU80, a SWI/SNF chromatin remodeling enzyme, TFII-i, and/or H2A.Z, as well as one or more post-translational modification. Functionally distinct CTCF complexes, e.g., distinguished by differences in cofactor interactions, can be found to associate with specific tumor suppressor genes. For example, a complex comprising CTCF and YB-1 can negatively regulate the transcription of c-myc (Chemukhin et al. (2000) “Physical and functional interaction between two pluripotent proteins, the Y-box DNA/RNA-binding factor, YB-1, and the multivalent zinc finger factor, CTCF.” J Biol Chem 275: 29915-29921), and a complex comprising CTCF and the chromodomain helicase protein CDH8 can form complexes that affect CpG methylation and histone acetylation proximal to the BRCA1 and c-myc genes (Ishihara et al. (2006) “CTCF-dependent chromatin insulator is linked to epigenetic remodeling.” Mol Cell 23: 733-742; Yusufzai et al. (2004) “CTCF tethers an insulator to subnuclear sites, suggesting shared insulator mechanisms across species.” Mol Cell 13: 291-298). In addition, where the gene is p16, an active CTCF complex can comprise a Topoisomerase IIβ. In addition, poly(ADP-ribosyl)ated CTCF has been found to bind to more than 140 mouse CTCF target sites (Yu et al. (2004) “Poly(ADP-ribosyl)ation regulates CTCF-dependent chromatin insulation.” Nat Genet. 36: 1105-1110). As described in the Example, active CTCF complexes comprise a PARlated CTCF polypeptide.

Accordingly, methods of identifying and characterizing additional protein components of CTCF complexes and the PARP-1 enzymatic machinery from a variety of target biological samples, e.g., those described below, are also a feature of the invention. CTCF and PARP-1 protein complexes can be isolated from cellular extracts derived from, e.g., one or more target biological samples described below, by gel filtration (Sephacel S-300) chromatography followed by Western analysis with CTCF or PARP-1 antibodies to detect the presence of either protein across the column. Alternately, such complexes can be immunoprecipitated directly from extracts derived from, e.g., target biological samples described below, using CTCF or PARP-1 antibodies. Complexes can then be electrophoresed on SDS-PAGE, individual protein bands can be excised, and individual members of the complexes can be identified by MALDI-TOF. Gel filtration is a gentle way to isolate multi-subunit protein complexes that might otherwise be unstable to high ionic strength buffers used in ion-exchange chromatography. Beneficially, the presence of different CTCF and PARP-1 complexes and their molecular weights from each biological sample can be identified. Moreover, gel filtration, or immunoprecipitation, coupled with MALDI-TOF analyses circumvent the need for epitope-tagging CTCF or PARP-1 enzymes, as epitope-tagging can interfere with subtle functions of the aforementioned complexes. Once the mass spec information is obtained, known interactors of either CTCF complexes or PARP-1 complexes can be verified by co-IP from cell extracts and Western analysis, using the appropriate antibodies.

As PARP-1 is known to be PARlated and phosphorylated, it is useful to characterize isolated PARP-1 complexes from each cell type for their post-translational modification status by Western analysis using antibodies to ADP-ribose polymers (PAR), phospho-serine-threonine, and phospho-tyrosine. The enzymatic activity of newly identified PARP-1 complexes can be assayed in vitro using published conditions with recombinant PARP-1 as a positive control (Guastafierro et al. (2008) “CCCTC-binding factor activates PARP-1 affecting DNA methylation machinery.” J Biol Chem 283: 21873-21880). These methods can reveal whether differences in the subunit composition or post-translational modification status of CTCF complexes and PARP-1 complexes exist between, e.g., p16 expressing and non-expressing human cancer cells, which may provide insight into the distinct PARlation reactivities towards CTCF.

Further Details Regarding Target Biological Samples

The invention provides methods, e.g., of identifying a modulator of a CTCF protein or a CTCF complex, of selecting a treatment for or determining the prognosis of a cancer-related disease, and of monitoring the cancer state of a cell. These methods each comprise monitoring an activity of a CTCF protein or CTCF complex in a biological or biochemical sample. The biological samples that can be used in various embodiments of these methods can include primary cells, e.g., cells obtained directly from a patient, e.g., from a tumor, or can include secondary cells, e.g., cells derived through the culture of cells obtained from a patient, or even well-known established cell lines, e.g., HeLa or other tumor cells.

Primary cells include cells that have been obtained directly from a human or veterinary patient, e.g., from a biopsy performed to obtain sample tissue and/or cultures thereof. Culturing primary cells in vitro can comprise disaggregating biopsy tissue, e.g., via proteolytic digestion, chemical disruption, and/or mechanical disruption. Because cell populations obtained from a biopsy can comprise more than one cell type(s), single cells can be isolated from an initial biological sample (tissue biopsy, blood sample, stool sample, sperm, urine sample, vaginal secretion, saliva, or the like), e.g., for use in the methods of the invention, e.g., with the cells being stored or grown, in an appropriate growth or storage media and can be incubated under appropriate environmental conditions (e.g., at an appropriate temperature and/or gas mixture in a sterile environment). Alternately, a mixed cell population can be fractionated, e.g., by cell type, e.g., via an appropriate flow cytometry method, e.g., fluorescence activated cell sorting (FACS), or by gravity sedimentation, centrifugation, sieving, and/or the like. Alternately, pieces of sterile biopsy tissue can be placed in growth media and incubated to produce an explant culture, and individual progenitor cells that migrate out of the explanted tissue onto the surface of the culture vessel can be transferred into fresh medium and cultured further.

Primary cell cultures are optionally formed from the cells that survive the desegregation process, attach to the cell culture vessel (and/or survive in suspension) and proliferate. Temperature, gas mixture, media composition, and other incubation conditions in which primary cell cultures are grown can vary and are typically optimized according to the source from which the biopsy was obtained, the type of tissue biopsied, the phenotype of the tissue, the cell's proliferative potential, the cell's nutritional requirements, or the like. Primary cells derived from, e.g., a tumor, a cancer cell of a patient, a multiple myeloma cell, a breast epithelial cancer cell, a cervical cancer cell, or the like, can be used in various embodiments of methods provided by the invention, e.g., methods of monitoring the cancer state of a cell, methods of selecting a treatment for or determining the prognosis of a cancer-related disease, and/or methods of identifying modulators of an activity of a CTCF protein or complex.

The cells in a primary culture are, typically, terminally differentiated, e.g., morphologically and physiologically similar to the parental tissues from which they were derived, and can retain the same capacity for biotransformation as the biopsied tissue. These characteristics make primary cell cultures desirable for use as biological samples in, e.g., methods of monitoring the cancer state of a cell and methods of selecting a treatment for or determining the prognosis of a cancer-related disease. However, with the exception of some cultures derived from tumors, most cultures of primary cells have a finite lifespan. In general, these cells will proliferate in culture for a limited number of cell divisions, e.g., depending on the source of the cell, the tissue type of the cell, and the like, after which they will senesce.

Further details regarding the facilities, reagents, and supplies for the maintenance and growth of primary cell cultures are detailed in, e.g., Schantz and Ng (2004) A Manual for Primary Human Cell Culture World World Scientific Publishing Company, Hackensack, N.J.; Freshney (2005) Culture of Animal Cells: A Manual of Basic Technique fifth edition, Wiley-Liss, New York; Boulton, Baker, and Walz (1992) Practical Cell Culture Technique, 1^{st} ed. Humana Press, NJ; and in Helgason and Miller (2004) Basic Cell Culture Protocols Springer-Verlag, NY. Media, equipment, and other cell culture reagents are commercially available from, e.g., Sigma Aldrich (St. Louis, Mo.), Invitrogen (Carlsbad, Calif.), and PromoCell (Heidelberg, Germany).

A secondary cell culture is typically derived through the culture of primary cells and which, in contrast to primary cell cultures, can divide and grow in culture for some time, e.g., 50-100 generations or more, before they senesce. Secondary cells can arise spontaneously in a primary cell culture, or, alternately, the establishment of a secondary cell line can be induced. Secondary cells can be distinguished from primary cells by a number of morphological, physiological and cytological criteria, including, e.g., abnormal chromosome number, loss of contact inhibition for adherent cells, shorter doubling times, an increase in the ratio of nuclear volume to cytoplasmic volume, etc. Using secondary cells, rather than primary cells, e.g., in the methods described herein, can be advantageous in that the technical difficulties of culturing and analyzing a homogenous population of primary cells can be reduced. Secondary cell lines, e.g., derived from a tumor or patient, provide a semi-renewable source of homogenous cells that can exhibit better retention of specialized functions than primary cells obtained from biopsy tissue. As such, cultures of secondary cells can also be beneficially used as biological samples in any one or more of the methods provided by the invention, e.g., methods of identifying modulators of an activity of a CTCF protein or complex, methods of selecting a treatment for or determining the prognosis of a cancer-related disease, and/or methods of monitoring the cancer state of a cell.

Individual cells in a secondary cell culture that grow more rapidly can eventually dominate a culture, thereby producing a population of cells that is less representative of the original tissue. Accordingly, it is useful for secondary cell cultures to be monitored to insure that the physiological, morphological, cytological, and metabolic characteristics of the cells in the secondary culture reflect those of the cells in the primary culture. Further details regarding the culture of secondary cells and the establishment of cell lines from such cells are elaborated in, e.g., the references cited above and in Langdon (2004) Cancer Cell Culture: Methods and Protocols Humana Press, NJ.

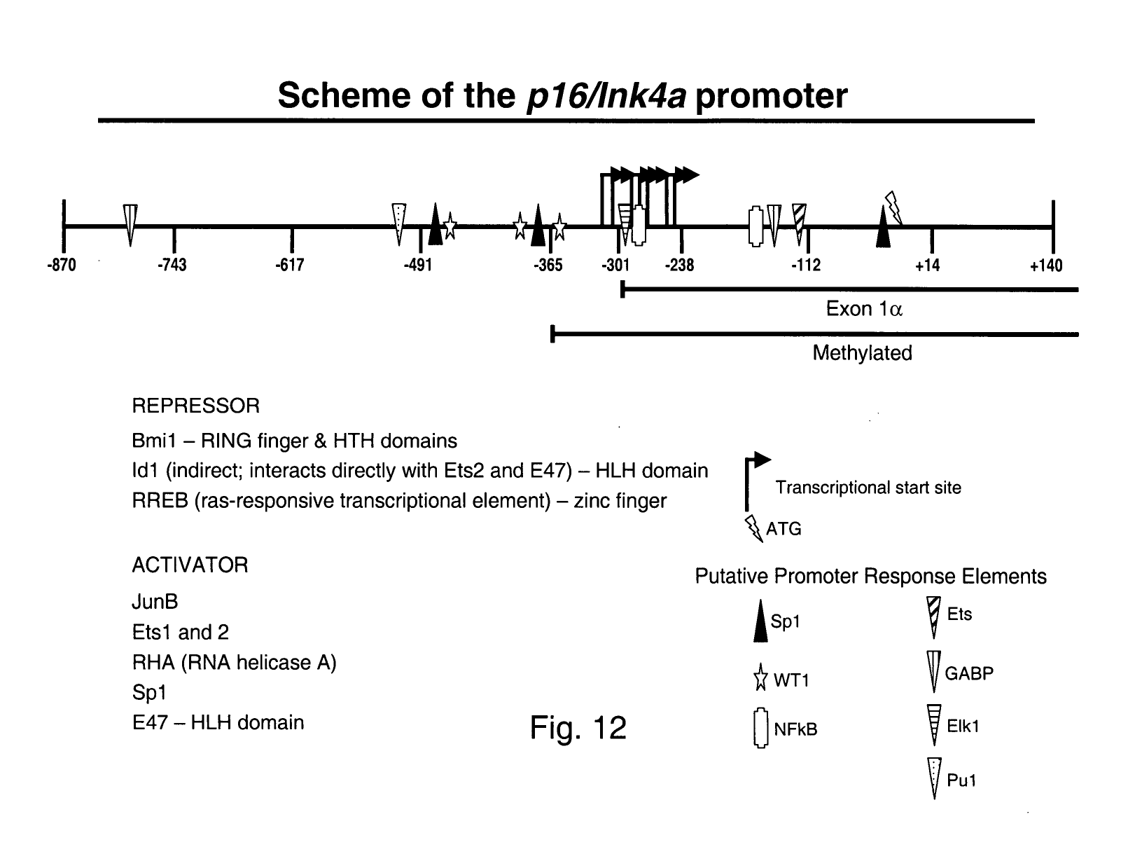

In addition, a variety of immortalized human cell lines, e.g., including, but not limited to U266, KMS12, TD47, MD-MB-435, vHMEC, HeLa, and IMR90, can be used with any one or more of the methods provided by the invention to detect an activity of a CTCF protein or CTCF complex. Cell lines can be selected, e.g., for use in the methods described elsewhere herein, on the basis of a variety of desirable criteria, including tissue type, pathology, genotypic properties, phenotypic properties such as proliferation rates, migration capacity, etc., or epigenetic properties, e.g., silenced or transcriptionally active p16 (a schematic of the p16 promoter and putative response elements involved in its transcriptional activation and repression are shown in FIG. 12). Such cell lines can continue to grow and divide indefinitely in vitro for as long as the correct culture conditions are maintained. Immortalized cell lines, e.g., those described above, are also known as transformed cells, e.g., cells whose growth properties have been altered via exposure to radiation, exposure to mutagens, infection with SV40 or polyomavirus, etc.

The cell lines described above can be obtained from ATCC (Manassas, Va.). Additional cell lines are also available from ATCC and from the World Federation for Culture Collections (Japan), the European Collection of Cell Cultures (from Sigma-Aldrich in St. Louis, Mo.) States and the National. Cancer Institute (Fredericksburg, Md.). A variety of cell lines are commercially available from, e.g., Invitrogen (Carlsbad, Calif.). Custom cell lines can also be produced by various commercial sources, e.g., ReaMetrix (San Carlos, Calif.) or GenWay Biotech, Inc. (San Diego, Calif.). Details regarding the culture and propagation of cell lines can be found in the references cited above, as well as in Ozturk and Hu (2005) Cell Culture Techniques for Pharmaceutical and Cell-Based Technologies CRC Press, Boca Raton, Fla., and in Lehr (2002) Cell Culture Models for biological Barrier: In Vitro Test Systems for Drug Absorption and Delivery CRC Press, Boca Raton, Fla.

### Screening Test Compounds for Modulators of an Activity of a CTCF Protein or CTCF Complex

Several drugs that reverse aberrant gene inactivation by modulating epigenetic processes, such as those that inhibit histone deacetylation or reverse DNA methylation have been identified. Unfortunately, these compounds are quite non-specific with regard to their targets and, even when combined, fail to stably restore gene function. Other targeted drugs, Gleevec (leukemia), Herceptin (breast cancer), Avastin and Erbitux (colon cancer) have been very beneficial; however, cancers often become resistant to these treatments, which generally must be applied with standard chemotherapies. In one aspect, the invention provides methods of identifying a compound that binds to or modulates an activity of a CTCF polypeptide (or complex). In these methods, a biological or biochemical sample comprising the polypeptide or complex is contacted with a test compound and binding of the test compound to the polypeptide or complex, or modulation of an activity of the polypeptide or complex by the test compound is detected, thereby identifying a CTCF modulator. Modulator compounds identified by these methods are also a feature of the invention.

Desirably, a modulator can be, e.g., a potentiator or enhancer of an activity of a CTCF polypeptide or complex, or an inhibitor of the CTCF polypeptide or complex. Such modulators can include, but are not limited to, polypeptides, e.g., phosphatase inhibitors, kinase inhibitors, small organic molecules, naturally occurring compounds, post-translational modification reagents, nucleotide analogs, nucleoside analogs, methylation reagents, hypomethylating nucleoside analogs, HDAC inhibitors, or the like. Modulators can include compounds that specifically bind to the CTCF polypeptide or complex. Modulators of interest can also include compounds that restore CTCF PARlation in human cancer cells and/or compounds that inhibit CTCF PARlation in non-cancerous cells. Such compounds can be tested for their ability to reestablish unstable chromosomal boundaries and reverse silencing of p16 and other deregulated genes to preserve genomic integrity, e.g., using any one or more of the screening formats described herein.

Compounds that modulate the activity of PAR polymerases and PARG hydrolases towards a variety of protein targets, e.g., other than CTCT, are also desirable.

Additional Details Regarding Assay Formats and Screening Methods

High throughput screening formats are particularly useful in identifying modulators of CTCF polypeptide (or complex) activity. Generally in these methods, one or more biological sample that includes a CTCF polypeptide or complex is contacted, serially or in parallel, with a plurality of test compounds comprising putative modulators (e.g., the members of a modulator library). Binding to or modulation of the activity of the polypeptide or complex by a test compound is detected, thereby identifying one or more modulator compound that binds to or modulates activity of the polypeptide, complex and/or gene.

Essentially any available compound library, e.g., a peptide library, a kinase inhibitor library, a phosphatase inhibitor library, a PARlation inducer library, a PARlation inhibitor library, or any one or combination of compound libraries described herein, can be screened to identify putative modulators in a high-throughput format against a biological or biochemical sample. As noted, the sample can include, e.g., a cancer cell, a multiple myeloma cell, a U266 cell, a KMS12 cell, a breast cancer cell, a TD47 cell, a primary breast epithelial cancer cell, a vHMEC, a MDA-MB-435 cell, a cervical cancer cell, a normal HMEC cell, a HeLa cell, a non-transformed fibroblast cell, an IMR90 cell, a primary cancer cell from a patient, or cell derived through culture from a primary cancer cell from a patient, and/or the like. The library members can then be assayed, optionally in a high-throughput fashion, for the ability to bind or modulate an activity of a CTCF polypeptide or complex.

Modulators of an activity of a CTCF protein or complex can optionally be identified, e.g., using the methods described herein, in, e.g., a combinatorial compound library. Such libraries typically include compounds sharing a common scaffold, with one or more scaffold substituents being varied (randomly or in a selected manner). The efficiency with which such modulators are identified can be optimized by prescreening or pre-selecting a library's constituents for desirable properties, e.g., oral availability, reduced toxicity, bioavailability, chemical structure, known activity, nuclear localization, ingestibility, and/or the like, to insure that compounds with the greatest potential for development, e.g., as therapeutic agents are highly represented in any library to be screened.

In a particular embodiment of the methods, a combinatorial compound library, e.g., a library comprising a variety of diverse, but structurally similar molecules synthesized by combinatorial chemistry methodologies, can be selected to comprise a majority of members that conform, e.g., to Lipinski's Rule of 5, a set of criteria by which the oral availability of a combinatorial compound can be evaluated. The rule states that an orally active drug, e.g., exhibiting desirable pharmacokinetic properties, will likely have i) no more than 5 hydrogen bond donors, ii) no more than 10 hydrogen bond acceptors, iii) a molecular weight under 500 g/mol, and iv) a partition coefficient log P less than 5, e.g., the compound will be lipophilic. Lipinski's Rule is useful in drug development and is typically applied at an early stage of drug design in order to select against putative modulators with poor absorption, distribution, metabolism, and excretion properties.

The efficiency of a screen to identify modulators of a CTCF protein or complex, e.g., in a combinatorial compound library, can also be enhanced by the use of in silico techniques to prioritize compounds with desirable characteristics, e.g., those described above, to be used in the methods provided herein, from the universe of compounds that can be synthesized and tested. For example, a ‘virtual library’, e.g., a computational enumeration of all possible structures with a given set of desirable biological properties, can be screened for promising candidates for use, e.g., in the methods described herein. For example, a pharmacophore can be used as a query to screen a database of compounds for molecules that share a distinct repertoire of structural and chemical features. As used herein, a “pharmacophore” is a three-dimensional configuration of steric and electronic properties common to all compounds that exhibit a particular biological activity.

Pharmacophore models are typically computationally-derived and are generally based on molecules, e.g., proteins, ligands, small organic compounds, and/or the like, that are known to bind the target of interest, e.g., a CTCF protein or complex, e.g., a CTCF complex comprising any one or more of a CTCF, a CHD8, a YB-1, a nucleophosmin, a Topoisomerase Ha, a Topoisomerase 1113, a Nucleolin, a Poly(ADP-ribose) polymerase (PARP1), an Importin alpha3/alpha1, a Lamin A/C, a YY-1, a DNA repair enzyme, a RAD50, an MRE11, an XRCC6/KU80, a SWI/SNF chromatin remodeling enzyme, and/or a TFII-i. Additional targets of interest to which, e.g., a protein, ligand, small organic molecule, and/or the like, can bind include, e.g., the product of a tumor suppressor gene, the promoter of a tumor suppressor gene, and/or a chromatin boundary.

Pharmacophore models developed in this manner can be refined using algorithms to search structural databases to identify ligands with similar three-dimensional features, which can have a greater-than-average probability of being active against the target, e.g., a CTCF protein or complex. Further details regarding pharmacophore identification are described in Khedkar et al. (2007) “Pharmacophore modeling in drug development and discovery: an overview.” Med Chem 3: 187-197; Reddy et al. (2007) “Virtual screening in drug discovery a computational perspective.” Curr Protein Pept Sci 8: 329-51; McInnes (2007) “Virtual screening strategies in drug discovery.” Curr Opin Chem Biol 11: 494-502; and Balakin et al. (2006) “Rational design approaches to chemical libraries for hit identification.” Curr Drug Discov Technol 3: 49-65.

Because a pharmacophore describes compounds based on their biological activity, using a pharmacophore to query a three-dimensional structure database can lead to the identification of new, structurally diverse candidate compounds, e.g., that can be synthesized and used in the methods described herein to identify modulators of an activity of a CTCF protein or complex. Computational screening can be most beneficial when a number of structurally diverse compounds, or “scaffolds”, are found for a given pharmacophore.

A combinatorial compound library can be based upon any number of scaffolds. For example, in some embodiments of the methods of identifying modulators of an activity of a CTCF protein or complex, a combinatorial compound library can optionally be based upon at least one pharmacophore scaffold. Alternately, a combinatorial compound library used in the methods can be based upon between, e.g., between about 1 and about 1000 or more different pharmacophore scaffolds, e.g., between about 1 and about 100 different pharmacophore scaffolds, e.g., up to about 45 different pharmacophore scaffolds, e.g., where each scaffold is represented in the library by a plurality of members. A combinatorial compound library can comprise any number of unique compounds, e.g., at least 500, at least 1000, at least 2500, at least 5000, at least 10,000, or at least 25,000 unique compounds. In one representative class of embodiments, the combinatorial compound library comprises at least about 4,000 unique compounds.

The number of members by which each scaffold can be represented is not particularly limited. Combinatorial compound libraries can conveniently be formatted into available micro-well plates such that each scaffold is represented by, e.g., 384 members or 96 members (or multiples thereof). Similarly, microfluidic or other available formats can be used, in which case the relevant library is formatted into arrays of members that fit available instrumentation.

In one convenient example embodiment, each scaffold can be represented by at least about 96 members, e.g., chemical variants that comprise the same basic chemical architecture as the scaffold, but which are each distinguished by unique side chains and R-groups. Including a wide variety of diverse scaffolds in an overall combinatorial compound library can improve the probability that a screen, e.g., to identify modulators of an activity of a CTCF protein or complex, will uncover desirable “lead” compounds, e.g., compounds with advantageous pharmacological and or biological properties whose chemical structures can be used as scaffolds for in vitro screens to, e.g., identify modulators of a CTCF protein or complex. Identifying multiple diverse desirable lead compounds can also be useful in managing the risk of compound attrition during subsequent screens to optimize potency, selectivity and/or pharmacokinetic properties, and during clinical development.

Various criteria, such as ADME (described in Balani et al. (2005) “Strategy of utilizing in vitro and in vivo ADME tools for leaf optimization and drug candidate selection.” Curr Top Med Chem 5: 1033-8), statistical methods, such as QSAR (described in Patani et al. (1996) “Bioisosterism: A Rational Approach in Drug Design.” Chem. Rev 96: 3147-3176 and Freyhult et al. (2003) “Structural modeling extends QSAR analysis of antibody-lysozyme interactions to 3D-QSAR.” J Biophys 84: 2264-2272), and algorithms, (reviewed in, e.g., Dror et al. (2006) “Predicting molecular interactions in silico: A guide to pharmacophore identification and its applications to drug design.” Curr Med Chem 11: 71-90), can be helpful in selecting the most beneficially useful compounds and scaffolds in a virtual library, e.g., of compounds that modulate the activity of a CTCF protein or complex, for actual synthesis. Other useful strategies for compound selection are described in, e.g., Olah et al. (2004) “Strategies for compound selection.” Curr Drug Discov Technol 1: 211-220.

Additional Details Regarding Libraries and Screening Platforms

In one aspect, the invention includes screening of libraries of modulator compounds, e.g., based upon pharmacophore models. Many three-dimensional structural databases of compounds, suitable for construction of pharmacophore compounds are commercially available, e.g., from the Sigma Chemical Company (Saint Louis, Mo.), Aldrich chemical company (St. Louis Mo.), Chembridge (San Diego, Calif.), Inte:Ligand (Austria), and others. Virtual compound library screening services can be performed by, e.g., Quantum Pharmaceuticals (Moscow, Russia), BIOMOL, and Chembridge, and others.

Libraries of synthesized compounds, which also may be screened for CTCF activity, are readily available, e.g., from TimTec (Newark, Del.), ArQule (Medford, Mass.), Exclusive Chemistry, LLC (Russia), and many others. Many companies, including those mentioned above, can custom synthesize compound libraries and/or offer library screening services, e.g., of proprietary compound libraries.

A variety of peptide libraries are commercially available from, e.g., Princeton BioMolecules (Langhorne, Pa.) and Cambridge Peptides (Cambridge, UK).

Kinase inhibitor libraries, phosphatase inhibitor libraries, and HDAC inhibitor libraries are available from EMD Biosciences (Germany), BIOMOL International (Plymouth Meeting, Pa.), TopoTarget (Denmark), and many others.

The source of modulator test compound for such systems and in the practice of the methods of the invention can optionally be any commercially available or proprietary library of materials, including compound libraries from the companies noted above, as well as typical compound and compound library suppliers such as Sigma (St. Louis Mo.), Aldrich (St. Louis Mo.), Agilent Technologies (Palo Alto, Calif.) or the like. The format of the library will vary depending on the system to be used. Libraries can be formatted in typical liquid phase arrays, e.g., using microtiter trays, can be formatted onto sets of beads, and/or can be formatted for microfluidic screening in either solid or liquid phase arrays.

Automated systems adapted to detection of CTCF activity can be used to assess any of a variety of relevant biological phenomena, including, e.g., expression levels of genes in response to selected stimuli (Service (1998) “Microchips Arrays Put DNA on the Spot.” Science 282: 396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like. Many such automated systems are commercially available and can be adapted to the detection of CTCF polypeptides. Examples of automated systems that can be adapted according to the invention include those from Caliper Technologies (including the former Zymark Corporation, Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). A number of automated approaches to high-throughput activity screening are provided by the Genomics Institute of the Novartis Foundation (La Jolla, Calif.); See GNF.org on the world-wide web. Microfluidic screening applications are also commercially available from Caliper Technologies Corp. For example, (e.g., LabMicrofluidic Device® high throughput screening system (HTS) by Caliper Technologies, Mountain View, Calif. or the HP/Agilent technologies Bioanalyzer using LabChip™ technology by Caliper Technologies Corp. can be adapted for use in the present invention.

In one illustrative embodiment, libraries of sample materials are arrayed in microwell plates (e.g., 96, 384 or more well plates), which can be accessed by standard fluid handling robotics, e.g., using a pipettor or other fluid handler with a standard ORCA robot (Optimized Robot for Chemical Analysis) available from Beckman Coulter (Fullerton, Calif.). Standard commercially available workstations such as the Caliper Life Sciences (Hopkinton, Mass.) Sciclone ALH 3000 workstation and Rapidplate™ 96/384 workstation provide precise 96 and 384-well fluid transfers in a small, highly scalable format. Plate management systems such as the Caliper Life Sciences Twister® II Advanced Capability Microplate Handler for End-Users, OEM's and Integrators provide plate handling, storage and management capabilities for fluid handling, while the Presto™ AutoStack provides fast reliable access to consumables presenting trays of tips, reagents, microplates or deep wells to an automated device (e.g., the ALH 3000) without robotic arm intervention.

In another illustrative embodiment, microfluidic systems for handling and analyzing microscale fluid samples, including cell based and non-cell based approaches that can be used for analysis of test compounds on biological samples in the present invention are also available, e.g., the Caliper Life Sciences various LabChip® technologies (e.g., LabChip® 90 and 3000) and related Agilent Technologies (Palo Alto, Calif.) 2100 and 5100 devices. Similarly, interface devices between microfluidic and standard plate handling technologies are also commercially available. For example, the Caliper Technologies LabChip® 3000 uses “sipper chips” as a “chip-to-world” interface that allows automated sampling from microtiter plates. To meet the needs of high-throughput environments, the LabChip® 3000 employs four or even twelve sippers on a single chip so that samples can be processed, in parallel, up to twelve at a time. Solid phase libraries of materials can also be conveniently accessed using sipper or pipetting technology, e.g., solid phase libraries can be gridded on a surface and dried for later rehydration with a sipper or pipette and accessed through the sipper or pipette.

As already noted, with regard to the systems and methods of the invention, the particular libraries of compounds can be any of those that now exist, e.g., those that are commercially available, or that are proprietary. A number of libraries of test compounds exist, e.g., those from Sigma (St. Louis Mo.), and Aldrich (St. Louis Mo.). Other current compound library providers include Actimol (Newark Del.), providing e.g., the Actiprobe 10 and Actiprobe 25 libraries of 10,000 and 25,000 compounds, respectively; BioMol (Philadelphia, Pa.), Enamine (Kiev, Ukraine) which produces custom libraries of billions of compounds from thousands of different building blocks, TimTec (Newark Del.), which produces general screening stock compound libraries containing >100,000 compounds, as well as template-based libraries with common heterocyclic lattices, libraries for targeted mechanism based selections, including kinase modulators, etc., privileged structure libraries that include compounds containing chemical motifs that are more frequently associated with higher biological activity than other structures, diversity libraries that include compounds pre-selected from available stocks of compounds with maximum chemical diversity, plant extract libraries, natural products and natural product-derived libraries, etc; AnalytiCon Discovery (Germany) including NatDiverse (natural product analogue screening compounds) and MEGAbolite (natural product screening compounds); Chembridge (San Diego, Calif.) including a wide array of targeted or general and custom or stock libraries; ChemDiv (San Diego, Calif.) providing a variety of compound diversity libraries including CombiLab and the International Diversity Collection; Comgenix (Hungary) including ActiVerse™ libraries; MicroSource (Gaylordsville, Conn.) including natural libraries, agro libraries, the NINDS custom library, the genesis plus library and others; Polyphor (Switzerland) including privileged core structures as well as novel scaffolds; Prestwick Chemical (Washington D.C.), including the Prestwick chemical collection and others that are pre-screened for biotolerance; Tripos (St. Louis, Mo.), including large lead screening libraries; and many others. Academic institutions such as the Zelinsky Institute of Organic Chemistry (Russian Federation) also provide libraries of considerable structural diversity that can be screened in the methods of the invention.

Additional Details Regarding Screening Formats

The activities of a CTCF protein and/or complex, e.g., the activities described above that can be monitored in methods provided by the invention, can be evaluated using any one or more well-known molecular biological techniques. For example, suppression or gene silencing of a tumor suppressor gene, or restoration of the expression of a tumor suppressor gene, e.g., a p16, a RASSF1a, a CDH1, and/or a c-Myc gene, can be assayed via standard mRNA quantitation assays, e.g., including, but not limited to northern blot analysis, reverse transcriptase coupled-polymerase chain reaction (RT-PCR), RNAse protection assays, and the like.

Northern blotting entails fractionating total RNA species on the basis of size by denaturing gel electrophoresis followed by transfer of the RNA onto a membrane by capillary, vacuum or pressure blotting. The RNA is then permanently bound to the membrane via exposure to short wave ultraviolet light or via exposure to heat at 80° C. in a vacuum oven. mRNA sequences of interest are detected on the blot by the hybridization of a specific, labeled nucleotide probe to the blot. Probes for northern blot detection generally contain full or partial cDNA sequences and may be labeled by enzymatic incorporation of ³²P- or ³³P-radiolabeled nucleotides or with nucleotides conjugated to haptens, e.g., biotin, for subsequent chemiluminescent detection. After probe hybridization, the blot is washed to remove nonspecific label. The hybridization signal is generally detected by exposing blots to X-ray film or phosphor storage plates, after prior incubation with chemiluminescent substrates, if necessary. The resulting position of the signal on the blot indicates the size of the mRNA to which the probe hybridized, and the intensity of the signal corresponds to the relative abundance of the mRNA of interest, indicating the expression level of, e.g., a p16, a RASSF1a, a CDH1, and/or a c-Myc gene in a biological sample of interest. Autoradiograph band intensities can be quantified by densitometry, by direct measurement of hybridized radiolabeled probe via storage phosphor imaging or by scintillation counting of excised bands.

Ribonuclease protection assays (RPAs) are also based on the hybridization of a labeled probe a target mRNA, e.g., a tumor suppressor gene mRNA. However, in the RPA, hybridization takes place in a solution containing both the target mRNA and the labeled probe, e.g., a probe that is complementary to the sequence of the target mRNA, without prior gel fractionation or blotting. After incubation for several hours, unhybridized probe and unhybridized sample RNA are enzymatically degraded and the remaining probe:RNA hybrids are electrophoresed through a denaturing polyacrylamide gel and visualized, e.g., by autoradiography or phosphorimaging. Alternatively, the RNase-resistant hybrids may be precipitated and bound to filters for direct quantitation by scintillation counting. Furthermore, by performing titration reactions with unlabeled RNA transcripts corresponding to the mRNA sense strand, absolute RNA levels in a sample of interest can be determined. RPA can offer at least 10-fold higher sensitivity than northern blot analysis, allowing the detection of low abundance mRNAs (see, e.g., Sambrook et al., Molecular Cloning—A Laboratory Manual (3rd Ed.), Vol. 1-3, Cold Spring Harbor Laboratory, Cold Spring Harbor, N.Y., 2000 (“Sambrook”)). The sensitivity and specificity of RPA can be attributed to the use of single-stranded RNA antisense probes which hybridize to a defined region of the target mRNA and are labeled to high specific activity.

The sensitivity of PCR has made RT-PCR useful in quantifying low abundance mRNA species and/or in detecting mRNAs of interest in small numbers of cells, e.g., primary cells from a tumor or patient, secondary cells derived from the culture of cells derived from a tumor or patient, or cells from an immortalized cell line. In short, RNA is harvested from biological sample(s) of interest, e.g., target samples described elsewhere herein, and optionally treated with DNAse. An mRNA species of interest, e.g., a p16, a RASSF1a, a CDH1, and/or a c-Myc mRNA, is then reverse transcribed into its DNA complement (cDNA), and the resulting cDNA is amplified using traditional PCR techniques, which are described further in Sambrook or Current Protocols in Molecular Biology, F. M. Ausubel et al., eds., Current Protocols, a joint venture between Greene Publishing Associates, Inc. and John Wiley & Sons, Inc. (“Ausubel”).

As the PCR amplification products accumulate, the exponential phase eventually enters a saturation phase where the products may approach similar levels irrespective of initial template concentration. Thus, in order for results to be meaningful, quantitative comparisons of amplified product are typically made during the exponential phase of a PCR reaction. In some implementations of RT-PCR, aliquots are removed from the PCR reaction following every few cycles, beginning at a point in the PCR reaction where product is undetectable, and extending through the entire exponential phase. Products are then resolved electrophoretically and quantitated by, e.g., densitometry, fluorescence or phosphorimaging.

In cases in which the tumor suppressor gene of interest encodes a transcription factor, e.g., p53, WT1, and others, reporter gene assays can be useful in monitoring CTCF protein or complex activity. For example, a reporter gene, e.g., CAT, lacZ, etc., can be site-specifically recombined into the genome of a cell of interest, e.g., any one or more of the cell types described in Details Regarding Target Biological Samples, downstream of a promoter of a tumor suppressor gene of interest via, e.g., Cre-Lox recombination (described in Sauer and Henderson (1988) “Site-specific DNA recombination in mammalian cells by the Cre 1 recombinase of bacteriophage P1.” Proc Natl Acad Sci USA 85: 5166-5170) or a similar system. Determining the activity of the reporter gene product can provide a direct and quantitative measurement of the level of transcription from the promoter of, e.g., a tumor suppressor gene of interest, and thereby indicate the ability of CTCF to stabilize, insulate, and/or maintain a chromosomal boundary proximal to the gene of interest.

In an alternate implementation, a reporter gene that has been placed under the transcriptional control of a constitutive promoter can be site-specifically recombined into a chromosomal locus, e.g., a transcriptionally active locus proximal to a chromosomal boundary maintained or stabilized by a CTCF protein or complex, as described above. Decreased reporter gene activity can indicate a defect in the ability of a CTCF protein or complex in preventing the spread of repressive nucleosomal modifications from a neighboring domain. This implementation can also be useful in a time course performed to track the reestablishment, or lack of reestablishment, of a chromatin domain boundary.

Any of the techniques described above can be used to measure the transcriptional expression or activity of a tumor suppressor gene, e.g., including, but not limited to a p16, a RASSF1a, a CDH1, and/or a c-Myc gene, thereby assaying the suppression, gene silencing, or restoration of tumor suppressor gene expression and providing a metric by which to monitor, e.g., the induction, or loss, of tumorigenesis in a biological sample of interest. Further details regarding primer design, probe production, reagents, and protocols for the aforementioned mRNA quantitation techniques, and other mRNA quantitation techniques, are described in Sambrook, Ausubel, Kaufman et al. (2003) Handbook of Molecular and Cellular Methods in Biology and Medicine Second Edition Ceske (ed) CRC Press (“Kaufman”); and The Nucleic Acid Protocols Handbook Ralph Rapley (ed) (2000) Cold Spring Harbor, Humana Press Inc (“Rapley”).

The binding of a CTCF protein or complex to a histone, a post-translationally modified histone, a chromatin, or a chromatin boundary, e.g., a chromatin boundary proximal to or within an INK4/ARF locus, a p16 gene, a RASSF1a gene, a CDH1 gene, and/or a c-Myc gene, is an activity that can be monitored via chromatin immunoprecipitation (ChIP). The activity of a CTCF protein or complex can also be indirectly monitored by using ChIP to analyze a variety of histone modifications within the vicinity of, e.g., p16 or other gene that is maintained in a transcriptionally active state by CTCF-mediated chromatin boundary formation. Histone modifications that can be analyzed, e.g., as an indirect measurement of CTCF protein or complex activity, include an increase or decrease in aberrant methylation, in H2A.Z binding, in the trimethylation of H3K4, in the monomethylation of H4K20, in the dimethylation of H3K27, or in the trimethylation of H3K9.

ChIP is based on the principle that DNA-bound proteins, e.g., a CTCF protein, a CTCF complex, or a histone modification proximal to or within a gene of interest, can be chemically crosslinked to the chromatin in living cells, e.g., primary cells derived from a patient or tumor, secondary cells derived from the culture of primary cells, or immortalized cell lines, thereby permitting the analysis of chromatin remodelling at chromosomal loci of interest. In addition, this assay provides another metric by which the induction or loss of tumorigenicity of a cell in a target sample can be evaluated

The crosslinking is usually accomplished by formaldehyde fixation, although it can be advantageous to use the reversible crosslinker DTBP. Following fixation, the cells are lysed and their DNA is sonicated to produce fragments that can be approximately 0.2-1 kb. Once the proteins are immobilized on the chromatin and the chromatin is fragmented, whole protein-DNA complexes can be immunoprecipitated using an an antibody specific for the protein in question, e.g., monomethylated H4K20, trimethylated H3K9, dimethylated H3K27, trimethylated H3K4, a CTCF protein or a CTCF complex that can optionally comprise any one or more of, e.g., CHD8, Topoisomerase IIα, Topoisomerase IIβ, Nucleolin, Nucleophosmin, Poly(ADP-ribose) polymerase (PARP1), Importin alpha3/alpha1, Lamin A/C, YB-1, YY1, a DNA repair enzyme, RAD50, MRE11, XRCC6/KU80, a SWI/SNF chromatin remodeling enzyme, TFII-i, and/or H2A.Z, as well as one or more post-translational modification. The DNA from the isolated protein/DNA fraction can then be purified, and the identity of the DNA fragments isolated in complex with, e.g., a CTCF protein or CTCF complex, can then be determined by PCR using primers specific for the DNA regions that the protein in question is hypothesized to bind.

Alternately, ChIP-on-chip, or ChIP-chip, analysis, e.g., chromatin immunoprecipitation using a DNA microarray, can be performed to determine where, e.g., a CTCF protein or CTCF complex, binds across the whole genome, thus permitting the characterization of a CTCF cistrome, e.g., the genome-wise set of cis-acting targets of a trans-acting factor such as a CTCF protein or CTCF complex. ChIP-sequencing, a system that combines ChIP with massively parallel DNA sequencing, used to map global genomic CTCF protein or CTCF complex binding sites in the genome of a target sample of interest, e.g., a primary cell derived from a tumor or a patient, a secondary cell derived from the culture of primary cells, or an immortalized cell Me, in a high-throughput, cost-effective fashion.

ChIP-chip analysis and/or ChIP-sequencing can assist a practitioner in determining whether a change in CTCF protein or complex activity is the result of a cis-acting defect or a trans-acting defect. For example, patients that display a lack of CTCF protein or complex binding proximal to or within a particular tumor suppressor gene, but normal CTCF protein or complex binding proximal to or within other tumor suppressor genes are likely to possess a cis-acting defect at the chromosomal locus at which CTCF binding is not detected. Patients that exhibit global, e.g., genome-wide, abberations in CTCF binding, e.g., relative to a normal subject, are likely to possess a trans-acting defect in the CTCF protein or complex. Such results can inform a diagnosis, or a disease prognosis, and can influence the selection of a treatment for a cancer-related disease or other disease arising from aberrant gene silencing.

Further details describing ChIP and ChIP-chip analysis are elaborated in Sambrook, in Ausubel, in Pells (2005) Nuclear Reprogramming: Methods and Protocols Humana Press, NJ, and in Negre et al. (2006) “Mapping the distribution of chromatin proteins by Chip in chip.” Methods in Enzymology (Elsevier, Amsterdam), Vol. 410: 315-41. Chip-sequencing is further described in Euskirschen et al. (2007) “Mapping of transcription factor binding regions in mammalian cells by ChIP: comparison of array- and sequencing-based technologies.” Genome Res 17: 898-909 and in Fredlake et al. (2008) “Ultrafast DNA sequencing on a microchip by a hybrid separation mechanism that gives 600 bases in 6.5 minutes.” Proc Natl Acad Sci USA 105: 476-81.

Another assay that can be performed to monitor the activity of a CTCF protein or CTCF complex, and to evaluate the induction, or loss, of tumorigenicity in a cell, is a time course tracking the reestablishment, or lack of reestablishment, of a chromatin domain boundary, e.g., a boundary that is maintained by the binding of a CTCF protein or complex, proximal to or within a gene of interest, e.g., p16 or other tumor suppressor gene. In this assay, a biological sample of interest, e.g., comprising primary cells from a tumor or a patient, secondary cells derived from a primary culture, or cells from an immortalized cell line, is treated with a hypomethylating-nucleoside analog, e.g., 5′AZA-2′-deoxycytidine (AZA), which reverses DNA methylation, permits chromatin remodeling, and reverses gene silencing. ChIP analysis can be performed on aliquots taken from the sample at designated time points following AZA treatment to monitor the recruitment and binding of a CTCF protein or complex. Failure to recruit a CTCF protein or complex can indicate advanced tumorigenicity in a cell, e.g., irreversible chromatin boundary instability proximal to or within, e.g., a tumor suppressor gene, and can also inform a diagnosis, a prognosis, or the selection of a treatment for cancer, a cancer-related disease, or an aging-related disease.

Functionally distinct CTCF complexes, e.g., distinguished by differences in cofactor interactions, can be found to associate with specific tumor suppressor genes. An active CTCF complex can optionally include any one or more CHD8, Topoisomerase Ha, Topoisomerase Nucleolin, Nucleophosmin, Poly(ADP-ribose) polymerase (PARP1), Importin alpha3/alpha1, Lamin A/C, YB-1, YY1, a DNA repair enzyme, RAD50, MRE11, XRCC6/KU80, a SWI/SNF chromatin remodeling enzyme, TFII-i, and/or H2A.Z, as well as one or more post-translational modification. (Methods of identifying additional members of a CTCF complex are described elsewhere herein.) Changes in chromatin boundary stabilization, insulation, or formation can be the result of defects in the formation of a specific CTCF complex near or in a tumor suppressor gene of interest. Distinct CTCF complexes can be distinguished, e.g., using antibody-based assays. For example, a CTCF complex can be immunoprecipitated from, e.g., lysates prepared from one or more target biological samples described elsewhere herein, using an antibody that recognizes the CTCF protein. Proteins bound to CTCF during its immunoprecipitation can then be eluted from the complex and analyzed (as described above).

In one implementation, western blotting can be used to identify proteins that can form a complex with CTCF. Western blotting entails separating the proteins that coimmunoprecipitate with CTCF via polyacrylamide gel electrophoresis (PAGE). The proteins are then transferred to a membrane, typically nitrocellulose or PVDF, which is then incubated with one or more antibody which can detect one or more target proteins of interest, e.g., proteins in the coimmmunoprecipitated complex other than CTCF. Other cofactors that coimmunoprecipitate with CTCF can also be identified via, e.g., mass spectrometry, protein microsequencing, etc.

To detect the PARlation state of a CTCF from one or more target biological samples, co-immunoprecipitations and/or westerns can be performed using an antibody specific for Poly(ADP-ribose) polymers.

### CTCF Screening for Diagnosis and Prognosis

An aspect of the invention is the discovery that CTCF binding at chromatin boundaries is useful for long-term gene expression for a variety of tumor suppressor genes. That is, the loss of CTCF/complex binding and/or the loss of CTCF PARlation coincide with gene silencing (e.g., tumor suppressor gene silencing) in multiple cancers.

Furthermore, while reversal of methylation by treatment with hypomethylation reagents such as AZA can lead to gene expression (e.g., of a tumor suppressor gene), this does not automatically lead to CTCF recruitment, and may lead to long term silencing. This lack of CTCF binding and/or CTCF PARlation after hypomethylation treatment can explain the inability to sustain long term expression of p16 and other tumor suppressors after reversal of epigenetic silencing by, e.g., AZA That is, failure to reestablish upstream chromatin boundaries by CTCF can lead to long term silencing.

This leads to a variety of diagnostic and prognostic assays. Specifically, the cancer state of a cell can be characterized in a variety of useful dimensions. First, the gene expression state of the cell can be characterized with respect to any of a variety of tumor suppressors, including an INK4/ARF gene locus, a p16^{INK4a} gene, a RASSF1a gene, a CDH1 gene or a C-Myc gene. Silencing of these tumor suppressors (or reduction in their level of expression compared to a control) provides an indication that the cancer cell is abnormal with respect to one or more of these genes. Second, the level of expression of CTCF and/or CTCF PARlation provides a similar indication (abnormal expression of CTCF and/or loss of CTCF PARlation can lead to gene misregulation, including tumor suppressor silencing). Third, abnormal methylation patterns within or proximal to a tumor suppressor gene provides an indication of epigenetic status of the gene. Fourth, binding of CTCF or complexes thereof, e.g., to chromatin boundaries proximal to or within a tumor suppressor gene provides a second indication of the epigenetic status of the gene.

The cancer state of a cell can also be additionally characterized in any of a variety of additional dimensions, e.g., by considering proliferative activity, expression or tumor markers, or any other cancer biology indicators that are currently in use.

Once the cancer cell has been characterized in one or more dimensions, e.g., including binding of CTCF and/or CTCF PARlation state, the information can be used in any of a variety of ways to assist the practitioner. For example, patients that are negative for CTCF binding to one or more tumor suppressor gene or proximal chromatin boundary and are negative for CTCF PARlation are at risk of long term gene silencing. If those same patient display lack of CTCF expression, then it can be possible to treat the disorder using gene therapy to deliver a CTCF-coding nucleic acid to the relevant cell, or by administering an agent that boosts CTCF expression. Similarly, if the patient displays normal CTCF expression and/or normal CTCF PARlation, but abnormal or reduced binding to several genes/chromatin boundaries, then a defect in expressed CTCF can be present; here again, treatment with a CTCF expression enhancing agent or gene therapy construct can be beneficial. Patients that display lack of CTCF binding to a particular tumor suppressor gene/region, but normal binding to other tumor suppressors most likely have a cis-defect in the relevant tumor suppressor. Treatment with a gene therapeutic that expresses the tumor suppressor and/or an agent that up regulates expression can be beneficial.

In addition to enhanced diagnostic capabilities, the information regarding cancer state is also prognostic of the cancer. For example, if a patient displays lack of CTCF binding and/or CTCF PARlation, and an epigenetic agent is administered that restores normal methylation to a gene or proximal region, it is useful to know whether CTCF binding and/or PARlation is restored. If CTCF binding and/or PARlation is restored, this can indicate that tumor suppressor expression can be restored longer term, providing an improved prognosis as compared to a patient that displays a lack of CTCF binding.

The ability to analyze cancer cell state in a multidimensional manner that takes account of CTCF binding status at one or more tumor suppressor genes or chromatin boundary regions also provides an ability to more specifically determine prognosis and to tailor treatment. In addition to the issues noted above, it is also possible to consider multidimensional data regarding CTCF binding, CTCF PARlation state, methylation, tumor suppressor expression, tumor marker expression and any other cancer state indicators in a statistical framework to improve the accuracy of diagnosis, prognosis, and treatment effects. Such multidimensional information can be fit into statistical and/or heuristic models to further refine diagnosis, prognosis, and treatment effects. For example, hidden Markov models (HMIVIs), partial least squares analysis, principle component analysis (PCA), projection to latent structures (PLS), genetic algorithms (GAs), and neural networks can all be used to assess multidimensional data and to refine correlations between CTCF binding and any other cancer state indicator and/or any combination of indictors and prognosis, diagnosis and treatment efficacy. Such statistical methods of correlating multidimensional data are well known and can be found, e.g., in Koski (2002) Hidden Markov Models of Bioinformatics (Computational Biology) Springer; 1st edition ISBN-10: 1402001363; Jones and Pevzner (2004) An Introduction to Bioinformatics Algorithms (Computational Molecular Biology) The MIT Press; 1st edition; Mount (2004) Bioinformatics: Sequence and Genome Analysis 2nd edition, ISBN-10: 0879697121; Eriksson et al. (2006) Multivariate and Megavariate Data Analysis Basic Principles and Applications (Parts I and II) Umetrics ISBN-10: 9197373028; Sivanandam and Deepa Introduction to Genetic Algorithms Springer; 1st edition ISBN-10: 354073189X.

Analysis of Differential Protein PARlation Profiles

Enzymatic defects in the PAR pathway that prevent the PARlation of CTCF, and, potentially, of other, as-yet unidentified critical protein targets, e.g., CTCF-associated cofactors, can contribute to the initiation of, e.g., human cancers and/or aging-related diseases, by deregulating chromosomal boundaries of tumor suppressor genes and silencing their expression. Thus, it can be useful to determine the protein PARlation profiles of samples derived from, e.g., target biological samples of interest (described elsewhere herein), to identify PARlation profiles that correlate with, e.g., cancer or aging-related diseases. Furthermore, such profiles can be of beneficial use not only in the diagnosis of such diseases, but also in their treatment and in the prediction of a prognosis. For example, the efficacy of a drug or agent that is administered to a patient diagnosed with, e.g., cancer or an aging-related disease, can be determined by whether or not the drug or agent restores a normal PARlation profile, e.g., in a biological sample derived from the patient. If a normal PARlation profile is restored, this can indicate an improved prognosis as compared to a patient that displays a lack of normal PARlation after the administration of the drug or agent.

On-Chip PARlation analyses can be performed to identify differential protein PARlation profiles from a variety of target biological samples. For example, a microarray that contains over 8,000 unique full-length recombinant human proteins that are expressed in a baculovirus system and purified under native conditions can be used in such an analysis. The proteins can be arrayed in duplicate on a nitrocellulose-coated glass slide (ProtoArray® Human Protein Microarray v. 4.0; Invitrogen), which slide also includes positive and negative PARlation controls. To identify protein targets of PARlation, cellular extracts from, e.g., target biological samples described elsewhere herein, can be incubated with the microarrays in the presence of fluorescent β-NAD⁺. The arrays can then be washed, dried, and scanned using commercially available fluorescent microarray scanners to measure incorporation of β-NAD⁺. This method can reveal the identity of PARlated proteins and the spectrum of PARP activity in a given sample. The protein PARlation profiles among the different biological samples examined can identify novel targets of this modification and reveal the frequency of loss of CTCF PARlation and whether other critical proteins are similarly affected.

This information can then be verified by immunoprecipitation of relevant tissue/cell extracts of CTCF and other known proteins, e.g., with the appropriate antibodies, followed by Western analysis with an anti-PAR antibody to confirm the PARlation status of the newly-identified target protein(s). If antibodies are not available, total cellular PARlated proteins can be immunoprecipitated with an anti-PAR antibody and resolved on SDS-PAGE. Individual proteins can then be excised and identified by MALDI-TOF.

### Recombinant Cells and Non-Human Animals

Transgenic Laboratory Animals

Transgenic (non-human) laboratory animals such as mice and other rodents are useful tools for studying gene function and for testing CTCF modulators. Human (or other selected) tumor suppressor genes can also be introduced in place of endogenous genes of a laboratory animal, making it possible to study function of the human (or other) tumor suppressor in the easily manipulated and studied laboratory animal. It will be appreciated that there is not precise correspondence between gene structure and function of different animals, making the ability to study the human or other tumor suppressor particularly useful. Although similar genetic manipulations can be performed in tissue culture, the interaction of tumor suppressors with recombinant, e.g., human CTCF and/or complexes thereof, in the context of an intact organism, provides a more complete and physiologically relevant picture of tumor suppressor/CTCF function than can be achieved in simple cell-based screening assays. Accordingly, one feature of the invention is the creation of transgenic animals comprising heterologous CTCF and/or tumor suppressor genes.

In general, such a transgenic animal is typically an animal that has had appropriate CTCF and/or tumor suppressor genes (or partial genes, e.g., comprising coding sequences coupled to a promoter) introduced into one or more of its cells artificially. This is most commonly done in one of two ways. First, a DNA encoding the relevant genes (or fragments thereof) can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing the (heterologous) DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted insertion of DNA is to make knock-out mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Further Details Regarding Cells Comprising CTCF and/or Tumor Suppressor Genes

As already noted, for several embodiments, biological samples to be tested for CTCF expression/activity, CTCF PARlation, or tumor suppressor expression/activity are cells or are derived from cell preparations. The cells can be those associated with CTCF or tumor suppressor expression (or lack thereof, e.g., cancer cells) in vivo. Alternately, the cells can be derived from such a cell, e.g., through primary or secondary culture.

However, one feature of the invention is the production of recombinant cells, e.g., expressing a heterologous CTCF and/or tumor suppressor gene. It is worth noting that recombinant cells expressing both recombinant CTCF and tumor suppressor genes such as a gene of an INK4/ARF gene locus, a p16^{INK4a} gene, a RASSF1a gene, a CDH1 gene or a C-Myc gene are a feature of the invention that arises out of the determination that CTCF regulates epigenetic programming of such genes, which was not previously known. Co-expression in a recombinant cell is particularly useful when screening for modulators of CTCF and/or tumor suppressor genes. By co-expressing CTCF (or complexes thereof) from a therapeutically relevant target (such as a human) along with a target tumor suppressor, it is possible to appropriately screen for activity in a model cell (chromosome position can also be controlled, e.g., using homologous recombination).

In these recombinant cell embodiments, the biological sample to be tested is derived from the recombinant cell, which is selected, e.g., for ease of culture and manipulation. The cells can be, e.g., human, rodent, insect, Xenopus, etc. and will typically be a cell in culture (or an oocyte in the case of Xenopus).

CTCF, CTCF complex and/or tumor suppressor nucleic acids are typically introduced into cells in cloning and/or expression vectors to facilitate introduction of the nucleic acid and expression of encoded proteins. Vectors can include, e.g., plasmids, cosmids, viruses, YACs, bacteria, poly-lysine, etc. A “vector nucleic acid” is a nucleic acid molecule into which a heterologous nucleic acid is optionally inserted that can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (e.g., in yeast and bacteria) artificial chromosomes. “Expression vectors” are vectors that comprise elements that provide for or facilitate the transcription of nucleic acids that are cloned into such vectors. Such elements can include, e.g., promoters and/or enhancers operably coupled to a nucleic acid of interest.

In general, appropriate expression vectors are known in the art. For example, pET-14b, pcDNA1 Amp, and pVL1392 are available from Novagen and Invitrogen and are suitable vectors for expression in E. coli, COS cells and baculovirus infected insect cells, respectively. pcDNA-3, pEAK, and vectors that permit the generation of PKD2L1 RNA for in vitro and in vivo expression experiments (e.g., in vitro translations and Xenopus oocyte injections) are also useful. These vectors are simply illustrative of those that are known in the art, with thousands of suitable vectors being available. Suitable host cells can be, e.g., any cell capable of growth in a suitable media and allowing purification of an expressed protein. Examples of suitable host cells include bacterial cells, such as E. coli, Streptococci, Staphylococci, Streptomyces and Bacillus subtilis cells; fungal cells such as yeast cells, Pichia, and Aspergillus cells; insect cells such as Drosophila S2 and Spodoptera Sf9 cells, mammalian cells such as CHO, COS, and HeLa; and even plant cells.

Cells are transformed with relevant genes (CTCF, tumor suppressor, etc.) according to standard cloning and transformation methods. Such genes can also be isolated from resulting recombinant cells using standard methods. General texts which describe molecular biological techniques for making nucleic acids, including the use of vectors, promoters and many other relevant topics, include Berger and Kimmel, Guide to Molecular Cloning Techniques, Methods in Enzymology volume 152 Academic Press, Inc., San Diego, Calif. (Berger); Sambrook; Ausubel; Kauman; and Rapley (above).

In addition, a plethora of kits are commercially available for the preparation, purification and cloning of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms, or the like.

As noted, typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Gillam & Smith (1979) “Site-specific mutagenesis using synthetic oligodeoxyribonucleotide primers: I. Optimum conditions and minimum oligodeoxyribonucleotide length.” Gene 8: 81-97; Roberts et al. (1987) “Generation of an antibody with enhanced affinity and specificity for its antigen by protein engineering.” Nature 328: 731-734; Schneider et al. (1995) “Functional Purification of a Bacterial ATP-Binding Cassette Transporter Protein (MalK) from the Cytoplasmic Fraction of an Overproducing Strain.” Protein Expr. Purif. 6435: 10-14; Ausubel, Sambrook, and Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others.

Other useful references, e.g., for cell isolation and culture (e.g., for subsequent nucleic acid isolation) include Freshney (2005) Culture of Animal Cells, a Manual of Basic Technique, fifth edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla.

RNAi and Antisense

In addition to increasing expression of CTCF as noted above (e.g., by expressing one or more recombinant copies of CTCF in a cell), expression or activity of endogenous CTCF or specific complex components can also be reduced. This can be accomplished using transcription factors (or inhibitors thereof) or, more typically, by using antisense or RNAi against the relevant transcript (e.g., directed to an mRNA of CTCF or a CTCF complex component). Thus, antisense and RNAi against CTCF or CTCF complex proteins represent one useful class of modulators that can be made and used in the present invention.

For example, the use of antisense nucleic acids is well known in the art. An antisense nucleic acid has a region of complementarity to a target nucleic acid, e.g., a target CTCF or CTCF complex protein's coding mRNA or DNA. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. No. 6,242,258 to Haselton and Alexander (Jun. 5, 2001) entitled, “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al. (1991) “Using antisense RNA to study gene function.” Symp Soc. Exp Biol 45: 117-127; de Lange et al. (1995) “Suppression of flavonoid flower pigmentation genes in Petunia hybrids by the introduction of antisense and sense genes.” Curr Top Microbiol Immunol 197: 57-75; Hamilton et al. (1995) “Sense and antisense inactivation of fruit ripening genes in tomato.” Curr Top Microbiol Immunol 197: 77-89; Finnegan et al. (1996) “Reduced DNA methylation in Arabidopsis thaliana results in abnormal plant development.” Proc Natl Acad Sci USA 93: 8449-8454; Uhlmann and Peyman (1990) “Antisense oligonucleotides: a new therapeutic principle.” Chem. Rev. 90: 543-584; P. D. Cook (1991) “Medicinal chemistry of antisense oligonucleotides—future opportunities.” Anti-Cancer Drug Design 6: 585-607; Goodchild (1990) “Conjugates of oligonucleotides and modified oligonucleotides: a review of their synthesis and properties” Bioconjugate Chem. 1: 165-187; Beaucage and Iyer (1993) “The synthesis of modified oligonucleotides by the phosphoramidite approach and their applications” Tetrahedron 49: 6123-6194; and F. Eckstein, Ed. (1991), Oligonucleotides and Analogues—A Practical Approach, IRL Press.

Gene expression can also be inhibited by RNA silencing or interference. “RNA silencing” refers to any mechanism through which the presence of a single-stranded or, more typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein.

The mechanism of RNAi has been and is being extensively investigated in a number of eukaryotic organisms and cell types. See, for example, the following reviews: McManus and Sharp (2002) “Gene silencing in mammals by small interfering RNAs.”Nature Reviews Genetics 3: 737-747; Hutvagner and Zamore (2002) “RNAi: Nature abhors a double strand.” Curr Opin Genet & Dev 200: 225-232; Hannon (2002) “RNA interference.” Nature 418: 244-251; Agami (2002) “RNAi and related mechanisms and their potential use for therapy.” Curr Opin Chem Biol 6: 829-834; Tuschl and Borkhardt (2002) “Small interfering RNAs: A revolutionary tool for the analysis of gene function and gene therapy.” Molecular Interventions 2: 158-167; Nishikura (2001) “A short primer on RNAi: RNA-directed RNA polymerase acts as a key catalyst.” Cell 107: 415-418; and Zamore (2001) “RNA interference: Listening to the sound of silence” Nature Structural Biology 8: 746-750. RNAi is also described in the patent literature; see, e.g., CA 2359180 by Kreutzer and Limmer entitled, “Method and medicament for inhibiting the expression of a given gene”; WO 01/68836 by Beach et al. entitled, “Methods and compositions for RNA interference”; WO 01/70949 by Graham et al. entitled, “Genetic silencing”; and WO 01/75164 by Tuschl et al. entitled, “RNA sequence-specific mediators of RNA interference.”

In brief, double-stranded RNA introduced into a cell (e.g., into the cytoplasm) is processed, for example by an RNAse III-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target gene in a cell can thus be specifically inhibited by introducing an appropriately chosen double-stranded RNA into the cell. Guidelines for design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czauderna et al. (2003) “Structural variations and stabilizing modifications of synthetic siRNAs in mammalian cells.” Nucl Acids Res 31: 2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems.” Proc. Natl. Acad. Sci. USA 98: 9742-9747; Elbashir et al. (2001) “Duplexes of 21-nucleotide RNAs mediate RNA interference in cultured mammalian cells.” Nature 411: 494-498; and Elbashir et al. (2002) “Analysis of gene function in somatic mammalian cells using small interfering RNAs.”Methods 26: 199-213 describe the use of 21 nucleotide siRNAs to specifically inhibit gene expression in mammalian cell lines, and Kim et al. (2005) “Synthetic dsRNA Dicer substrates enhance RNAi potency and efficacy.” Nature Biotechnology 23: 222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 by (base pairs) of complementarity between the antisense strand and the target mRNA in the context of a 21 mer with a 19 by double-stranded region has been shown to result in a functional siRNA (see, e.g., Czauderna et al. (2003) “Structural variations and stabilizing modifications of synthetic siRNAs in mammalian cells.” Nucl Acids Res 31: 2705-2716). Any overhangs can but need not be complementary to the target mRNA; for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi.” Cell 110: 563-574; Amarzguioui et al. (2003) “Tolerance for mutations and chemical modifications in a siRNA.” Nucl. Acids Res. 31: 589-595; Holen et al. (2003) “Similar behavior of single-strand and double-strand siRNAs suggests that they act through a common RNAi pathway.” Nucl. Acids Res. 31: 2401-2407; and Schwarz et al. (2002) “Evidence that siRNAs Function as Guides, Not Primers, in the Drosophila and Human RNAi Pathways.” Mol. Cell 10: 537-548.

Due to differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers.” Nature Biotechnology 23: 227-231).

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing.” Science 301: 1069-1074; Kawasaki and Taira (2004) “Induction of DNA methylation and gene silencing by short interfering RNAs in human cells.” Nature 431: 211-217; and Morris et al. (2004) “Small interfering RNA-induced transcriptional gene silencing in human cells.” Science 305: 1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA. The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms.” Proc. Natl. Acad. Sci. USA 100: 9779-9784; Doench et al. (2003) “siRNAs can function as miRNAs.” Genes & Dev. 17: 438-442; Bartel and Bartel (2003) “MicroRNAs: At the root of plant development?” Plant Physiology 132: 709-717; Schwarz and Zamore (2002) “Why do miRNAs live in the miRNP?” Genes & Dev. 16: 1025-1031; Tang et al. (2003) “A biochemical framework for RNA silencing in plants.” Genes & Dev. 17: 49-63; Meister et al. (2004) “Sequence-specific inhibition of microRNA- and siRNA-induced RNA silencing.” RNA 10: 544-550; Nelson et al. (2003). “The microRNA world: Small is mighty.” Trends Biochem. Sci. 28: 534-540; Scacheri et al. (2004) “Short interfering RNAs can induce unexpected and divergent changes in the levels of untargeted proteins in mammalian cells.” Proc. Natl. Acad. Sci. USA 101: 1892-1897; Sempere et al. (2004) “Expression profiling of mammalian microRNAs uncovers a subset of brain-expressed microRNAs with possible roles in murine and human neuronal differentiation.” Genome Biology 5: R13; Dykxhoorn et al. (2003) “Killing the messenger: Short RNAs that silence gene expression.” Nature Reviews Molec. and Cell Biol. 4: 457-467; McManus (2003) “MicroRNAs and cancer.” Semin Cancer Biol. 13: 253-288; and Stark et al. (2003) “Identification of Drosophila microRNA targets.” PLoS Biol. 1: E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression.” Genes Dev. 18: 504-511; Rehmsmeier et al. (2004) “Fast and effective prediction of microRNA/target duplexes.” RNA 10: 1507-1517; Robins et al. (2005) “Incorporating structure to predict microRNA targets.” Proc Natl Acad Sci USA 102: 4006-4009; and Mattick and Makunin (2005) “Small regulatory RNAs in mammals.” Hum. Mol. Genet. 14: R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against the target mRNA to determine which is most effective at repressing translation of the target mRNA.

Additional Details Regarding Protein Purification and Handling

Purification of CTCF and/or complexes thereof, tumor suppressor proteins, or the like can be accomplished using known techniques. In one embodiment, transformed cells expressing such proteins are lysed, crude purification occurs to remove debris and some contaminating proteins, followed by chromatography to further purify the protein to the desired level of purity. Such purified components can be used in modulator screening assays (e.g., to detect modulator binding), to raise antibodies against the proteins (e.g., for in situ labeling, or as modulators), and the like.

Cells can be lysed by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, polypeptides can be purified, either partially (e.g., achieving a 5×, 10×, 100×, 500×, or 1000× or greater purification), or even substantially to homogeneity (e.g., where the protein is the main component of a solution, typically excluding the solvent (e.g., water or DMSO) and buffer components (e.g., salts and stabilizers) that the polypeptide is suspended in, e.g., if the polypeptide is in a liquid phase), according to standard procedures known to and used by those of skill in the art. Accordingly, polypeptides of the invention can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against the relevant polypeptide (CTCF, complex, tumor suppressor, or the like) are used as purification reagents, e.g., for affinity-based purification. Once purified, partially or to homogeneity, as desired, the polypeptides are optionally used e.g., as assay components (e.g., to test putative modulators), as therapeutic reagents or as immunogens for antibody production.

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

Those of skill in the art will recognize that, after synthesis, expression and/or purification, proteins can possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art (see, the references above, and Debinski et al. (1993) “A wide range of human cancers express interleukin 4 (IL4) receptors that can be targeted with chimeric toxin composed of IL4 and Pseudomonas exotoxin.” J. Biol. Chem. 268: 14065-14070; Kreitman and Pastan (1993) “Purification and characterization of IL6-PE4e, a recombinant fusion of interleukin 6 with Pseudomonas exotoxin.” Bioconjug. Chem. 4: 581-585; and Büchner et al. (1992) “A method for increasing the yield of properly folded recombinant fusion proteins: single-chain immunotoxins from renaturation of bacterial inclusion bodies.” Anal. Biochem. 205: 263-270). Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

CTCF, CTCF complex and/or tumor suppressor nucleic acids optionally comprise a coding sequence fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, et al. (1984) “The structure of an antigenic determinant in a protein.” Cell 37: 767-778), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the sequence of the invention is useful to facilitate purification.

Antibodies to CTCF, Complexes and Tumor Suppressors

Antibodies against CTCF, CTCF complexes (or individual components thereof), methylated proteins (histones, etc.), tumor suppressors, and/or the like, are available, or can be made using available methods. Such antibodies are useful in the methods as noted herein, and/or as affinity purification reagents. Antibodies can optionally discriminate between different CTCF complexes or different post translational modifications. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein. General details regarding the production of antibodies can be found, e.g., in Howard and Kaser (Eds) (2006) Making and Using Antibodies: A Practical Handbook CRC; 1st edition ISBN-10: 0849335280; Maher Albitar (2007) Monoclonal Antibodies: Methods and Protocols (Methods in Molecular Biology); Humana Press; 1st edition ISBN-10: 158829567; and Zola (1999) Monoclonal Antibodies Basics (Bios Scientific Publishers). Garland Science; 1st edition ISBN-10: 1859960928, as well as in a variety of modern textbooks such as Paul (ed) (2008) Fundamental Immunology Lippincott Williams & Wilkins; 6th edition # ISBN-10: 0781765196.

For example, in the production of antibodies to any of the components noted (CTCF, complexes, tumor suppressors, etc.), various host animals may be immunized by injection with the relevant component, or a portion thereof. Such host animals include, but are not limited to, rabbits, mice and rats. Various adjuvants may be used to enhance the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guérin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique, which provides for the production of antibody molecules by continuous cell lines in culture. These include, but are not limited to, the hybridoma technique of Kohler and Milstein (1975) “Continuous cultures of fused cells secreting antibody of predefined specificity.” Nature 256: 495-497 and U.S. Pat. No. 4,376,110; the human B-cell hybridoma technique (described in Kosbor et al. (1983) “The Production of Monoclonal Antibodies from Human Lymphocytes.”Immunology Today 4: 72-79 and Cote et al. (1983) “Generation of human monoclonal antibodies reactive with cellular antigens.” Proc. Nat'l. Acad. Sci. USA 80: 2026-2030), and the EBV-hybridoma technique (Cole et al., Monoclonal Antibodies and Cancer Therapy, Alan R. Liss, Inc., pp. 77-96, 1985). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. (1984) “Chimeric human antibody molecules: mouse antigen-binding domains with human constant region domains.” Proc. Nat'l. Acad. Sci. USA 81: 6851-6855; Neuberger et al. (1984) “Recombinant antibodies possessing novel effector functions.” Nature 312: 604-608; Takeda et al. (1985) “Construction of chimaeric processed immunoglobulin genes containing mouse variable and human constant region sequences.”Nature 314: 452-454) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird et al (1988) “Single-chain antigen-binding proteins.” Science 242: 423-426; Huston et al. (1988) “Protein engineering of antibody binding sites: recovery of specific activity in an anti-digoxin single-chain Fv analogue produced in Escherichia coli.” Proc. Nat'l. Acad. Sci. USA 85: 5879-5883; and Ward et al. (1989) “Binding activities of a repertoire of single immunoglobulin variable domains secreted from E. coli.” Nature 341: 544-546) can be adapted to produce differentially expressed gene-single chain antibodies. Single chain antibodies are formed by linking the heavy and light chain fragments of the Fv region via an amino acid bridge, resulting in a single-chain polypeptide.

In one aspect, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al. (1989) “Generation of a large combinatorial library of the immunoglobulin repertoire in phage lambda.” Science 246: 1275-1281) to allow rapid and easy identification of monoclonal Fab fragments with the desired specificity.

Protocols for detecting and measuring the expression of CTCF and/or complexes, and/or tumor suppressors as noted herein, using the above mentioned antibodies, can be performed according to methods well known in the art. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

One method, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody that is specific for the protein expressed by the gene of interest.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of PLAB that is present in the serum sample.

Alternately, fluorescent compounds, such as fluorescein and rhodamine, can be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

### Cell Rescue-Treatment

In one aspect, the invention includes rescue of a cell that is defective in function of one or more endogenous CTCF, CTCF complex, or tumor suppressor gene(s), polypeptides or complexes thereof. This can be accomplished simply by introducing a new copy of the gene(s) (or a heterologous nucleic acid(s) that expresses the relevant protein(s)) into a cell. Other approaches, such as homologous recombination to repair a defective gene (e.g., via chimeraplasty) can also be performed. In any event, rescue of function can be measured, e.g., in any of the assays noted herein. Indeed, this can be used as a general method of screening cells in vitro for activity. Accordingly, in vitro rescue of function is useful in this context for the myriad in vitro screening methods noted above, e.g., for the identification of modulators in cells. The cells that are rescued can include cells in culture, (including primary or secondary cell culture from patients, as well as cultures of well-established cells). Where the cells are isolated from a patient, this has additional diagnostic utility in establishing which sequence is defective in a patient that presents with, e.g., a cancer, and/or to determine whether the defect is a cis- or a trans-defect.

In another aspect, gene rescue occurs in a patient, e.g., a human or veterinary patient, e.g., to remedy a genetic or epigenetic defect. Thus, one aspect of the invention is gene therapy to remedy tumor suppressor expression defects, in human or veterinary applications. In these applications, the nucleic acids of the invention are optionally cloned into appropriate gene therapy vectors (and/or are simply delivered as naked or liposome-conjugated nucleic acids), which are then delivered (site-specifically, e.g., to a tumor, or, optionally systemically), optionally in combination with appropriate carriers or delivery agents. Proteins can also be delivered directly, but delivery of the nucleic acid is typically preferred in applications where stable expression is desired.

Vectors for administration typically comprise CTCF, CTCF complex or tumor suppressor genes under the control of a promoter that is expressed in target cells. These can include, e.g., native promoters (e.g., for CTCF, a tumor suppressor such as p16^{INK4a} gene, a RASSF1a gene, a CDH1 gene or a C-Myc, or other cell-specific promoters that are known to be active in the target cell.

Compositions for administration, e.g., comprise a therapeutically effective amount of the gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention.

Therapeutic compositions comprising one or more nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells of interest (taste bud, tongue, palate epithelium, neuronal cells, kidney cells, etc.). Practitioners can select an administration route of interest based on the cell target. For example, topical administration (e.g., for skin cancers) or direct injection into tumors is simplest and therefore can be preferred for these targets. However, systemic introduction, e.g., using target cell-specific vectors can also be performed. Suitable methods of administering such nucleic acids in the context of the present invention to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there are a wide variety of suitable formulations of pharmaceutical compositions of the present invention. Compositions can be administered by a number of routes including, but not limited to: oral, intravenous, intraperitoneal, intramuscular, transdermal, subcutaneous, topical, sublingual, spinal or rectal administration. Compositions can be administered via liposomes (e.g., topically), or via topical delivery of naked DNA or viral vectors. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

The dose administered to a patient, in the context of the present invention, is sufficient to effect a beneficial therapeutic response in the patient over time, or, e.g., to provide sweet or glutamate tastant discrimination as perceived by the patient in an objective sweet or glutamate tastant test. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression in the taste buds, or circulating plasma levels, formulation toxicities, progression of the relevant disease, and/or where relevant, the production of antibodies to proteins encoded by the polynucleotides. The dose administered, e.g., to a 70 kilogram patient, is typically in the range equivalent to dosages of currently-used therapeutic proteins, adjusted for the altered activity or serum half-life of the relevant composition. The vectors of this invention can supplement treatment conditions by any known conventional therapy (e.g., diet restriction, etc.).

For administration, formulations of the present invention are administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

### Further Details Regarding Term Definitions

As used herein, an aging-related disease or an age-related disorder refers to a disease that is seen with increasing frequency with advanced age, e.g., during organismal senescence. Aging-related diseases are not necessarilty a consequence of the ageing process itself, as not all adults experience all age-associated diseases. Examples of aging-related diseases include, e.g., Alzheimer's disease, Parkinson's disease, Huntington's disease, cardiovascular disease, diabetes mellitus, metabolic syndrome, dementia, senile dementia, many cancers, and others.

As used herein, a biological or biochemical sample comprising the CTCF polypeptide or a CTCF polypeptide complex polypeptide includes any sample comprising the polypeptide or polypeptide complex that is derived from a biological source, e.g., cells, tissues, organisms, etc. These samples can include, e.g., cells expressing the polypeptides or complexes, lysates or cell extracts containing the polypeptides or complexes, polypeptides or complexes bound to a chemical matrix, polypeptides or complexes bound to solid surface (e.g., for plasmon resonance), etc. A biochemical source can include biological sources and/or non-biological sources, such as purely synthetic preparations of materials.

As used herein, a CTCF polypeptide is a polypeptide that is the same as a naturally occurring CTCF protein (sometimes termed “CCCTC binding factor”), or a polypeptide that is homologous to such a naturally occurring CTCF protein (e.g., a protein derived from a CTCF protein through mutation or artificial manipulation). Naturally occurring CTCFs include conserved zinc finger polypeptides that bind DNA and that can act, e.g., as noted herein. A variety of splicing variants and mutants are known and characterized and are included within the meaning of the term, unless context indicates otherwise. A CTCF polypeptide complex is a complex that forms between CTCF and other polypeptides (e.g., described elsewhere herein) or nucleic acids (or both), e.g., at or proximal to a promoter or a chromatin boundary. FIGS. 13 and 24 provide additional details regarding various CTCF binding partners.

As used herein, “Lipinski's Rule of 5” refers to a set of criteria by which the oral availability of a combinatorial compound can be evaluated. The rule states that an orally active drug, e.g., exhibiting desirable pharmacokinetic properties, will likely have i) no more than 5 hydrogen bond donors, ii) no more than 10 hydrogen bond acceptors, iii) a molecular weight under 500 g/mol, and iv) a partition coefficient log P less than 5, e.g., the compound will be lipophilic. Lipinski's Rule is useful in drug development and is typically applied at an early stage of drug design in to select against putative modulators with poor absorption, distribution, metabolism, and excretion properties.

As used herein, a “modulator” is a compound that modulates an activity of a CTCF polypeptide or CTCF polypeptide complex. The term “modulate” with respect to a CTCF polypeptide or complex refers to a change in an activity or property of the polypeptide or complex. For example, modulation can cause an increase or a decrease in a protein or complex activity, a binding characteristic, or any other biological, functional, or immunological properties of a CTCF protein or complex. The change in activity can arise from, for example, an increase or decrease in expression of one or more genes that encode these proteins, the stability of an mRNA that encodes the protein, translation efficiency, or from a change in activity of the protein itself. For example, a molecule that binds to a CTCF polypeptide or complex can cause an increase or decrease in a biological activity of the polypeptide or complex.

As used herein, “PARlation” refers to a post-translational protein modification that is produced by ADP-ribosyltransferase enzymes, which transfer the ADP-ribose group from nicotinamide adenine dinucleotide (NAD^{±}) onto acceptors such as arginine, glutamic acid or aspartic acid residues in their substrate protein. ADP-ribose can also be transferred to proteins in long branched chains, in a reaction called poly(ADP-ribosyl)ation. This protein modification is carried out by the poly ADP-ribose polymerases (PARPs) which are found in most eukaryotes, but not prokaryotes or yeast. Poly(ADP-ribosyl)ation regulates multiple biological processes including DNA repair, genotoxic stress, telomere maintenance, and epigenetic programming by post-translational modification of critical regulatory proteins and chromatin components (reviewed in D′Amours et al. (1999) “Poly(ADP-ribosyl)ation reactions in the regulation of nuclear functions.” Biochem J 342: 249-268 and Burkle (2005) “Poly(ADP-ribose): The most elaborate metabolite of NAD⁺.” FEBS J 272: 4576-4589; Kraus (2008) “Transcriptional control of PARP-1: chromatin modulation, enhancer-binding, coregulation, and insulation.” Curr Opin Cell Biol 20: 294-302; and Klenova and Ohlsson (2005) “Poly(ADP-ribosyl)ation and epigenetics. Is CTCF PARt of the plot?” Cell Cycle 4:96-101.) The β-NAD+-dependent addition of ADP-ribose polymers to a variety of proteins, e.g., including p53, NF-□B, and CTCF, is catalyzed by the abundant, ubiquitous nuclear enzyme PARP-1.

As used herein, a “pharmacophore” refers to a three-dimensional configuration of steric and electronic properties common to all compounds that exhibit a particular biological activity. Pharmacophore models are typically computationally-derived and are generally based on molecules, e.g., proteins, ligands, small organic compounds, and/or the like, that are known to bind the target of interest.

As used herein, a “prescreened” compound is a compound that is pre-selected for a property of interest, such as toxicity, lack of toxicity, bioavaliability, chemical structure, type of molecule (kinase inhibitor, phosphatase inhibitor, post-translational modification reagent, nucleoside analog, nucleotide analog, methylation reagent, hypomethylating nucleoside analog, HDAC inhibitor, polypeptide, a naturally occurring compound, a small organic molecule, etc.), or the like.

As used herein, a “scaffold” refers to one of the structurally diverse chemcial compounds that comprise a pharmacophore. A chemical scaffold is typically the common structural subunit of a given family of molecules, e.g., a combinatorial compound library, wherein each member of the family comprises the same basic chemical architecture as the scaffold, but is distinguished by unique side chains and R-groups

### EXAMPLE

The following example is offered to illustrate, but not limit, the claimed invention. One of skill will recognize a variety of non-critical parameters that can be modified to achieve essentially similar results.

### Overview

The p16^{INK4a} tumor suppressor gene is a frequent target of epigenetic inactivation in human cancers, which is considered to be an early event in breast carcinogenesis. Here we describe the existence of a chromatin boundary upstream of the p16^{INK4a} gene that is lost when this gene is aberrantly silenced. We show that the multifunctional protein CTCF associates in the vicinity of this boundary and that loss of CTCF binding strongly coincides with p16^{INK4a} silencing in multiple types of cancer cells. A causal role for CTCF in epigenetic programming and activation of the p16^{INK4a} gene is demonstrated by CTCF knockdown experiments. CTCF binding also correlates with activation of the RASSF1A and CDH1 genes, and this interaction is absent when these genes are methylated and silenced. Interestingly, defective poly(ADP-ribosyl)ation of CTCF and dissociation from the molecular chaperone Nucleolin occurs in p16-silenced cells, abrogating its proper function. Thus, destabilization of specific chromosomal boundaries through aberrant crosstalk between CTCF, poly(ADP-ribosyl)ation, and DNA methylation may be a general mechanism to inactivate tumor suppressor genes and initiate tumorigenesis in numerous forms of human cancers.

Introduction

Aberrant transcriptional silencing of tumor suppressor genes by epigenetic deregulation is a common occurrence in human malignancies. This is characterized by altered patterns of DNA hypermethylation in specific promoter regions and acquisition of histone modifications that are characteristic of repressed chromatin, such as deacetylation of histones 3 and 4 and methylation of specific lysine residues like H3K9 and H3K27 (Feinberg et al. (2006) “The epigenetic progenitor origin of human cancer.” Nat Rev Genet. 7: 21-33; Feinberg (2008) “Epigenetics at the epicenter of modern medicine.” JAMA 299: 1345-1350; Jenuwein (2006) “The epigenetic magic of histone lysine methylation.” FEBS J 273: 3121-3135; Jones and Baylin (2007) “The epigenomics of cancer.” Cell 128: 683-692). Transcriptional inactivation and chromatin repression usually precede DNA hypermethylation at silenced promoters (Bachman et al. (2003) “Histone modifications and silencing prior to DNA methylation of a tumor suppressor gene.” Cancer Cell 3: 89-95; Strunnikova et al. (2005) “Chromatin inactivation precedes de novo DNA methylation during the progressive epigenetic silencing of the RASSF1A promoter.” Mol Cell Biol 25: 3923-3933). Several seminal studies have revealed insights into the interplay between methyl-DNA binding proteins, such as MeCP2, and complexes with histone deacetylase or methyltransferase activities indicating that these proteins act in concert to form repressive chromatin structures through targeted recruitment to specific promoters (Harikrishnan et al. (2005) “Brahma links the SWI/SNF chromatin-remodeling complex with MeCP2-dependent transcriptional silencing.” Nat Genet. 37: 254-264; Jones et al. (1998) “Methylated DNA and MeCP2 recruit histone deacetylase to repress transcription.” Nat Genet. 19: 187-191; Nan et al. (1998) “Transcriptional repression by the methyl-CpG-binding protein MeCP2 involves a histone deacetylase complex.” Nature 393: 386-389; Zhang et al. (1999) “Analysis of the NuRD subunits reveals a histone deacetylase core complex and a connection with DNA methylation.” Genes Dev 13: 1924-1935).

Because of its importance in cell proliferation, the human INK4 gene locus is a frequent target of inactivation by deletion or aberrant DNA methylation in a wide variety of human cancers (Kim and Sharpless (2006) “The regulation of INK4/ARF in cancer and aging.” Cell 127: 265-275; Lowe and Sherr (2003) “Tumor suppression by Ink4a-Arf: progress and puzzles.” Curr Opin Genet Dev 13: 77-83). This locus encompasses approximately 42 kb on chromosome 9 and encodes three distinct tumor suppressor proteins, p15^{INK4b}, p14^{ARF} and p16^{INK4a} (referred to hereafter as p15, p14 and p16). p16 is a key regulator of G1 phase cell cycle arrest and senescence, which it achieves primarily through inhibiting the cyclin-dependent kinases CDK4 and CDK6. Inactivation of these CDKs maintains Rb in a hypophosphorylated form enabling it to repress genes required for transition to S phase. In fact, inactivation of the p16 gene by promoter methylation or genetic change is one of the earliest losses of tumor suppressor function in numerous types of human cancers, such as breast, lung, colorectal cancers and multiple myeloma (Belinsky et al. (1998) “Aberrant methylation of p16(INK4a) is an early event in lung cancer and a potential biomarker for early diagnosis.” Proc Natl Acad Sci USA 95: 11891-11896; Esteller et al. (2001) “A gene hypermethylation profile of human cancer.” Cancer Res 61: 3225-3229; Foster et al. (1998) “Inactivation of p16 in human mammary epithelial cells by CpG island methylation.” Mol Cell Biol 18: 1793-1801; Ng et al (1997) “Frequent hypermethylation of p16 and p15 genes in multiple myeloma.” Blood 89: 2500-2506). Notably, p16 promoter methylation and transcriptional silencing have been shown to exist in histologically normal mammary tissue of cancer-free women. This suggests that these aberrant epigenetic changes may represent a cancerous pre-condition and an early event in promoting genomic instability that leads to tumorigenesis (Hoist et al. (2003) “Methylation of p16(INK4a) promoters occurs in vivo in histologically normal human mammary epithelia.” Cancer Res 63: 1596-1601).

Although the precise mechanisms underlying epigenetic loss-of-function of the p16 gene remain unresolved, an examination of proteins important for its regulation may provide insight into the cause of aberrant silencing. The transcription factors Ets, JunB and Sp1 have each been shown to directly activate p16 expression through cis elements (Ohtani et al. (2001) “Opposing effects of ETS and ID proteins on p16INK4a expression during cellular senescence.” Nature 409: 1067-1070; Passegue and Wagner (2000) “JunB suppresses cell proliferation by transcriptional activation of p16(INK4a) expression.” EMBO J. 19: 2969-2979; Wu et al. (2007) “Sp1 is essential for p16 expression in human diploid fibroblasts during senescence.” PLoS ONE 2: e164). Therefore, dysfunction or aberrant recruitment of repressor complexes by these activators could result in p16 gene inactivation. However, at present little evidence exists that these factors are deregulated at the p16 promoter in cancer cells. The p38 signaling cascade has also been demonstrated to regulate p16 expression, albeit through an unknown mechanism (Bulavin et al. (2004) “Inactivation of the Wip1 phosphatase inhibits mammary tumorigenesis through p38 MAPK-mediated activation of the p16(Ink4a)-p19(Arf) pathway.” Nat Genet. 36: 343-350). Constitutive activation of p38 in mice null for the phosphatase Wip-1 significantly reduces tumor formation in mouse models of breast cancer. Furthermore, this reduction is abrogated by deletion of the p16 and p14 genes. Thus, p38 signaling serves as a potential therapeutic target whereby p16 could be reactivated by stimulation of this pathway. One study revealed that transcription of all three INK4/ARF genes is controlled by a common Cdc6-binding regulatory element (Gonzalez et al. (2006) “Oncogenic activity of Cdc6 through repression of the INK4/ARF locus.” Nature 440: 702-706). When this putative origin-of-replication is heterochromatinized by targeted RNA-interference, transcriptional repression of the three tumor suppressor genes ensues. Moreover, the oncogenic activity of Cdc6 is greatly reduced in INK4/ARP^{-/-} MEFs and a reciprocal abundance of p16 and Cdc6 proteins is found in non-small cell lung carcinomas. However, inactivation of the INK4/ARF locus through one governing element is unlikely to be the sole cause of aberrant silencing of these tumor suppressors. This is supported by RNA expression and DNA methylation profiles of p15, p14 and p16 genes in a variety of tumors and cancer cell lines that show no coupling of p16 silencing with the two other genes (Bisogna et al. (2001) “Molecular analysis of the INK4A and INK4B gene loci in human breast cancer cell lines and primary carcinomas.”Cancer Genet Cytogenet 125: 131-138; Paz et al. (2003) “A systematic profile of DNA methylation in human cancer cell lines.” Cancer Res 63: 1114-1121).

p16 silencing could also result from gain-of-function or aberrant targeting of repressor proteins that modulate epigenetic processes. In this regard, several known repressors of the p16 gene may be involved. For example, the ID family member IDI plays a critical role in p16 regulation during senescence in human fibroblasts through exchange of ID for ETS activators (Ohtani et al. (2001) “Opposing effects of ETS and ID proteins on p16INK4a expression during cellular senescence.” Nature 409: 1067-1070). However, it is unclear if this contributes to p16 deregulation during tumorigenesis. Another repressor of p16, the polycomb group member BMI1, has been shown to have oncogenic activity (Haupt et al. (1991) “Novel zinc finger gene implicated as myc collaborator by retrovirally accelerated lymphomagenesis in E mu-myc transgenic mice.” Cell 65: 753-763; van Lohuizen et al. (1991) “Identification of cooperating oncogenes in E mu-myc transgenic mice by provirus tagging.” Cell 65: 737-752) and to control cell proliferation and senescence through the INK4a locus (Jacobs et al. (1999) “The oncogene and Polycomb-group gene BMI1 regulates cell proliferation and senescence through the ink4a locus.”Nature 397: 164-168; Smith et al. (2003) “BMI1 regulation of INK4A-ARF is a downstream requirement for transformation of hematopoietic progenitors by E2a-Pbx1.” Mol Cell 12: 393-400). BMI1 directly interacts with the p16 gene and maintains low levels of its expression in early passage proliferating fibroblasts while in senescent cells BMI1 association is lost. In primary breast tumors, however, no correlation between BMIland p16 expression is observed (Silva et al. (2006) “Implication of polycomb members BMI1, MeI-18, and Hpc-2 in the regulation of p16INK4a, p14ARF, h-TERT, and c-Myc expression in primary breast carcinomas.” Clin Cancer Res 12: 6929-6936). Other polycomb members such as EZH2 and SUZ12 also interact with p16 in proliferating fibroblasts (Bracken et al. (2007) “The Polycomb group proteins bind throughout the INK4A-ARF locus and are disassociated in senescent cells.” Genes Dev 21: 525-530; Kotake et al. (2007) “pRB family proteins are required for H3K27 trimethylation and Polycomb repression complexes binding to and silencing p16INK4alpha tumor suppressor gene.” Genes Dev 21: 49-54). Recent evidence indicates that EZH2 can recruit DNA methyltransferases to target promoters and maintain methylation patterns at silenced genes in cancer cells (Vire et al. (2006) “The Polycomb group protein EZH2 directly controls DNA methylation.” Nature 439: 871-874). As EZH2 was shown to bind the p16 gene in fibroblasts, deregulation of EZH2 may represent a direct link to epigenetic changes occurring at the p16 locus during oncogenesis.

To further understand the mechanism(s) by which the p16 gene becomes aberrantly silenced in human cancers, we examined epigenetic regulation at the level of the INK4/ARF chromosomal locus rather than solely at the p16 promoter. Here we present evidence that a chromosomal boundary exists at approximately 2 kb upstream of the p16 transcriptional start site. This boundary separates the p16 gene locus into discrete domains characterized by the presence or absence of repressive epigenetic marks and the histone variant H2A.Z, a functionally diverse protein recently shown to confer memory of transcriptional status and facilitate re-activation of target promoters (Brickner et al. (2007) “H2A.Z-Mediated Localization of Genes at the Nuclear Periphery Confers Epigenetic Memory of Previous Transcriptional State.” PLoS Biol 5: e81; Guillemette and Gaudreau (2006) “Reuniting the contrasting functions of H2A.Z.” Biochem Cell Biol 84: 528-535; Raisner and Madhani (2006) “Patterning chromatin: form and function for H2A.Z variant nucleosomes.” Curr Opin Genet Dev 16: 119-124). By contrast, in breast cancer cells containing aberrantly silenced p16 genes, the epigenetically defined domain at −2 kb disappears and regions 3′ of this boundary acquire characteristics of heterochromatin, which is accompanied by loss of histone H2A.Z. Upon further examination, we noticed the presence of a recognition sequence for the zinc finger protein CTCF 3′ of the boundary. CTCF is a multi-functional transcription factor known to have a critical role in regulating chromosomal boundaries/insulators (Filippova (2008) “Genetics and epigenetics of the multifunctional protein CTCF.” Curr Top Dev Biol 80: 337-360; Recillas-Targa et al. (2006) “Epigenetic boundaries of tumour suppressor gene promoters: the CTCF connection and its role in carcinogenesis.” J Cell Mol Med 10: 554-568; Wallace and Felsenfeld (2007) “We gather together: insulators and genome organization.” Curr Opin Genet Dev 17: 400-407). Unexpectedly, we observed CTCF association with this region in numerous p16-expressing cell lines but complete absence in p16 non-expressing breast cancer cells, even though CTCF binds to another target, the c-Myc gene, in all cases. Moreover, ablation of CTCF protein from p16-expressing cells by shRNA results in epigenetic changes to the p16 promoter and loss of transcription. In addition to breast cancer, aberrant p16 gene silencing is widely documented in a variety of human malignancies. We examined multiple myeloma cell lines and found that inactivation of the p16 gene is also correlated with absence of CTCF binding. Thus, our studies indicate that p16 gene repression can result from destabilization of a chromosomal boundary through dissociation of CTCF.

A similar examination of other well characterized epigenetically silenced genes in human cancers, RASSF1A and CDH1 (E-cadherin), also revealed a strong correlation between transcriptional inactivation and a loss of CTCF binding. The insulator function of CTCF has been shown to require its post-translational modification by poly(ADP-ribosyl)ation (PARlation) (Yu et al. (2004) “Poly(ADP-ribosylation regulates CTCF-dependent chromatin insulation.” Nat Genet. 36: 1105-1110) and crosstalk between PARP-1 and CTCF strongly affects DNA methylation (Guastafierro et al. (2008) “CCCTC-binding factor activates PARP-1 affecting DNA methylation machinery.” J Biol Chem 283: 21873-21880). Strikingly, we found a defect in the poly(ADP-ribosyl)ation pathway in p16-silenced cells resulting in the absence of CTCF PARlation and dissociation from a new coregulator, Nucleolin. Furthermore, we demonstrated that chemical inhibition of PARlation or knockdown of PARP-1 directly impacts p16 and RASSF1A expression. We propose that destabilization of specific chromosomal boundaries is caused by aberrant interactions between CTCF and the poly(ADP-ribosyl)ation enzymatic machinery and can be a general mechanism to initiate potentially reversible genomic instability and tumorigenesis in human cancers and aging-related diseases.

Loss of a Chromosomal Boundary at the p16 Gene Locus in Epigenetically Silenced Breast Cancer Cells

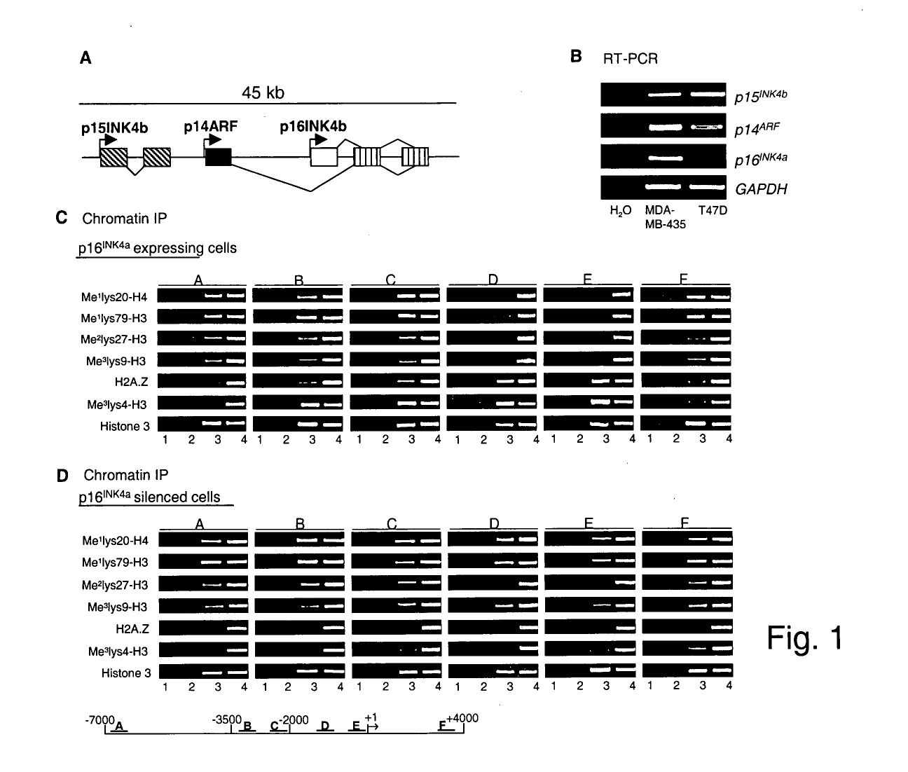

Aberrant transcriptional silencing of tumor suppressor genes is accompanied by dynamic changes in chromatin structure as revealed by the acquisition of histone modifications that are characteristic of repressed chromatin. To gain insight into chromatin structural alterations that may accompany p16 gene inactivation, we analyzed histone modifications surrounding the gene in p16-expressing (MDA-MB-435) and non-expressing (T47D) human breast cancer cell lines (FIG. 1). Initially, we examined the transcriptional status of the three genes within the INK4/ARF locus, p15, p14, and p16 (diagrammed in FIG. 1A). We found that each gene is active in MDA-MB-435 cells whereas p16 alone is silenced in T47D cells (See FIG. 1B, which shows RT-PCR analysis of gene expression at the INK4locus in MDA-MB-435 cells, e.g., p16-expressing cells, and T47D breast cancer cells, e.g., p16-non-expressing cells.) This indicates that event(s) leading to p16 deregulation in these cells can specifically impact this gene without affecting the entire INK4/ARF locus.

We next performed chromatin immunoprecipitations (ChiPs) to analyze a variety of histone modifications within the vicinity of the active p16 gene in MDA-MB-435 cells. These modifications include those that are typically associated with repressed chromatin, like monomethylated H4K20, dimethylated H3K27, and trimethylated H3K9, as well as marks that correlate with mammalian gene activation, such as trimethylated H3K4 and the histone variant H2A.Z. We also examined the presence of monomethylated H3K79 which is generally correlated with transcriptionally active genes in mammalian cells (Klose and Zhang (2007) “Regulation of histone methylation by demethylimination and demethylation.” Nat Rev Mol Cell Biol 8: 307-318). Localization of bulk histone 3 was measured to control for any large changes in nucleosomal placement and density. ChIP analyses of histone modifications surrounding the p16 gene in p16-expressing cells are shown in FIG. 1C (Lanes: 1. H2O; 2. no antibody; 3. antibodies to various histone modifications; 4. 1.6% total input DNA). ChIP-enriched DNA was PCR-amplified using specific amplicons (A-F) distributed throughout the p16gene locus. Surprisingly, in p16-expressing cells, we found an enrichment of marks that are normally associated with silenced genes, as well as monomethylated H3K79, between 2-7 kb upstream of the proximal promoter (FIG. 1C; amplicons A-C, FIG. 19). This chromatin structural organization is lost in the vicinity of the p16 proximal promoter between −2 kb and +1 (amplicons D-E). As expected in this region of an expressed gene, trimethylated H3K4 is enriched and H2A.Z is distributed in a similar pattern. The region of “active” chromatin between −2 kb and +1 is reversed downstream of the p16 gene at +4 kb where chromatin again becomes repressed (amplicon F). These data indicate that the 11 kb region encompassing the p16 gene is arranged into clearly demarcated domains of repressive versus active chromatin structures. Moreover, the data are consistent with the presence of a distinct chromosomal boundary/insulator within 2 kb upstream of the p16 transcriptional start site.

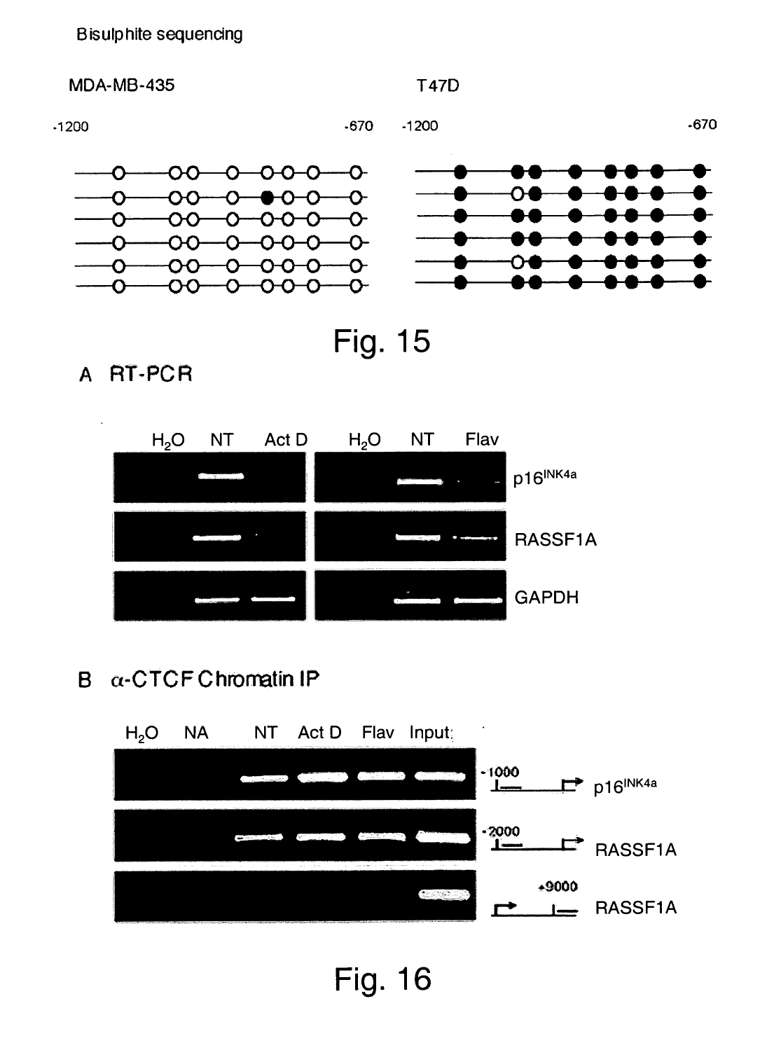

A similar ChIP analysis was conducted in T47D breast cancer cells in which the p16 gene is silenced and methylated (FIG. 15) (Di Vinci et al. (2005) “p16(INK4a) promoter methylation and protein expression in breast fibroadenoma and carcinoma.” Int J Cancer 114: 414-421). Methylation analysis of the region upstream of p16 was performed. FIG. 15 provides a schematic diagram showing the results of bisulphate sequencing of the CTCF-associated region upstream of the p16 gene in MDA-MB-435 and T47D cells. In this figure, DNA from p16-expressing and non-expressing human breast cancer cell lines have been subjected to bisulphite sequencing to identify DNA sequences in the p16 promoter region that are methylated at cytosine-guanine (CG) bases. DNA methylation of p16 and of genes in general is correlated with gene silencing. In the data shown in FIG. 15, each circle represents a CpG dinucleotide. An open circle means that the cytosine residue is not methylated, whereas a filled circle indicates that the cytosine of this particular CpG is methylated. In these cells, the chromatin structure of the aberrantly inactivated p16 gene is quite different from that found in p16-expressing MDA-MB-435 cells and, most strikingly, the chromosomal domain organization is lost (FIG. 1D). This is apparent from the spread of repressive histone marks, monomethylated H4K20 and trimethylated H3K9 as well as monomethylated H3K79 from the upstream 2-7 kb domain through the −2 kb demarcation to encompass the entire 11 kb p16 gene locus. A dramatic loss of H2A.Z and trimethylated H3K4 from approximately −3 kb to +1 (amplicons B-E) is also evident. However, no significant change in the pattern of dimethylated H3K27 is observed, indicating that the enzymatic activity associated with this mark can function in an independent manner. Overall, these data substantiate the existence of a chromatin boundary upstream of the p16 initiation site that functions to maintain the promoter in an active configuration by preventing the spread of repressive nucleosomal modifications from a neighboring domain. Interestingly, the disappearance of this boundary is correlated with aberrant epigenetic silencing of the p16 gene in certain breast cancer cell lines.

The Boundary/Insulator Protein CTCF Associates with the Transcriptionally Active but not Silenced p16 Gene

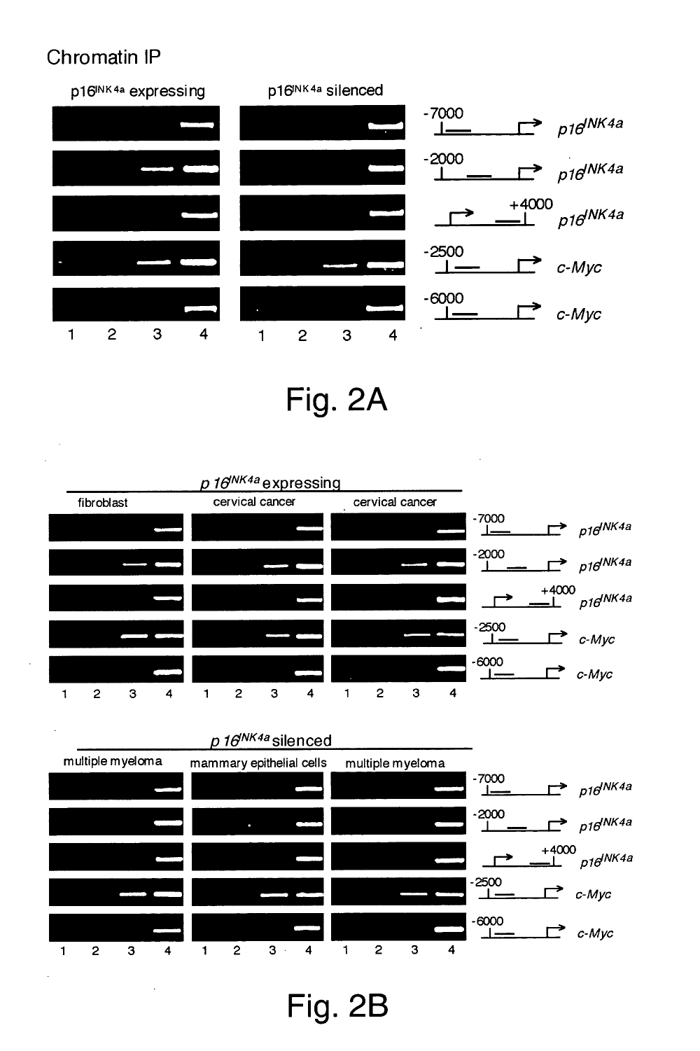

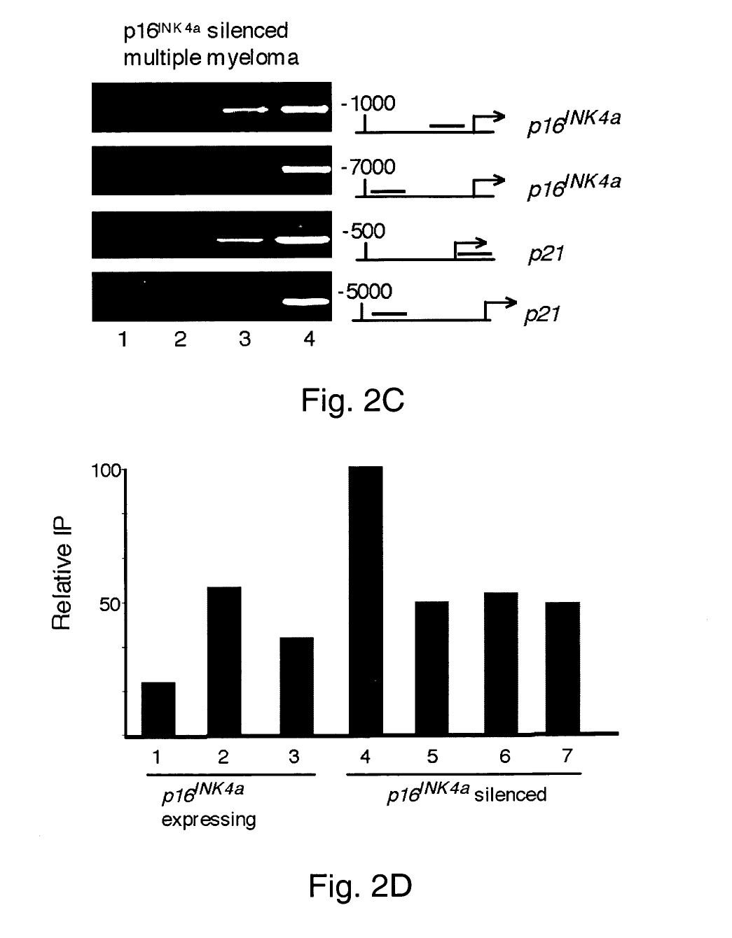

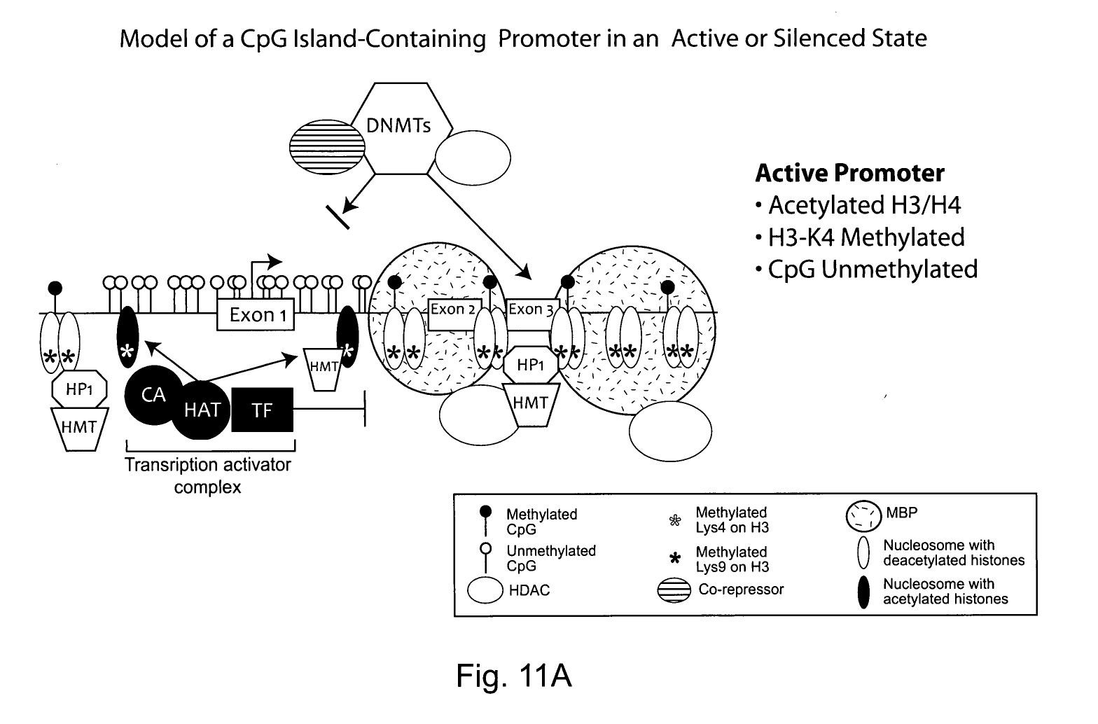

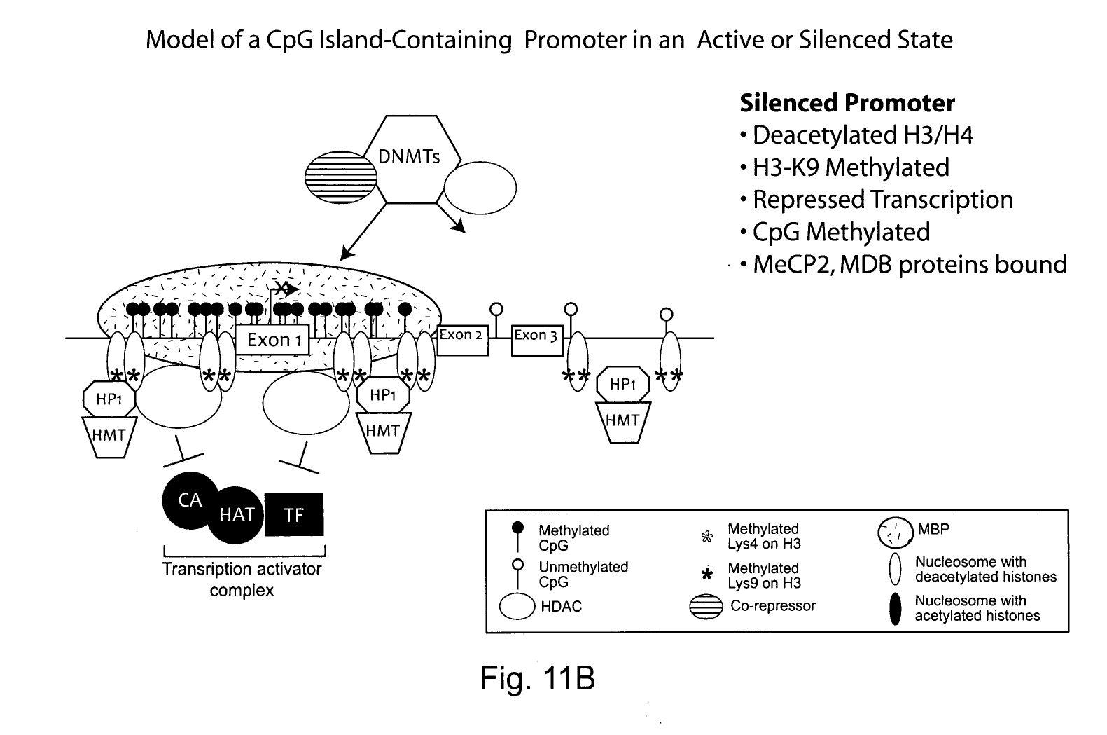

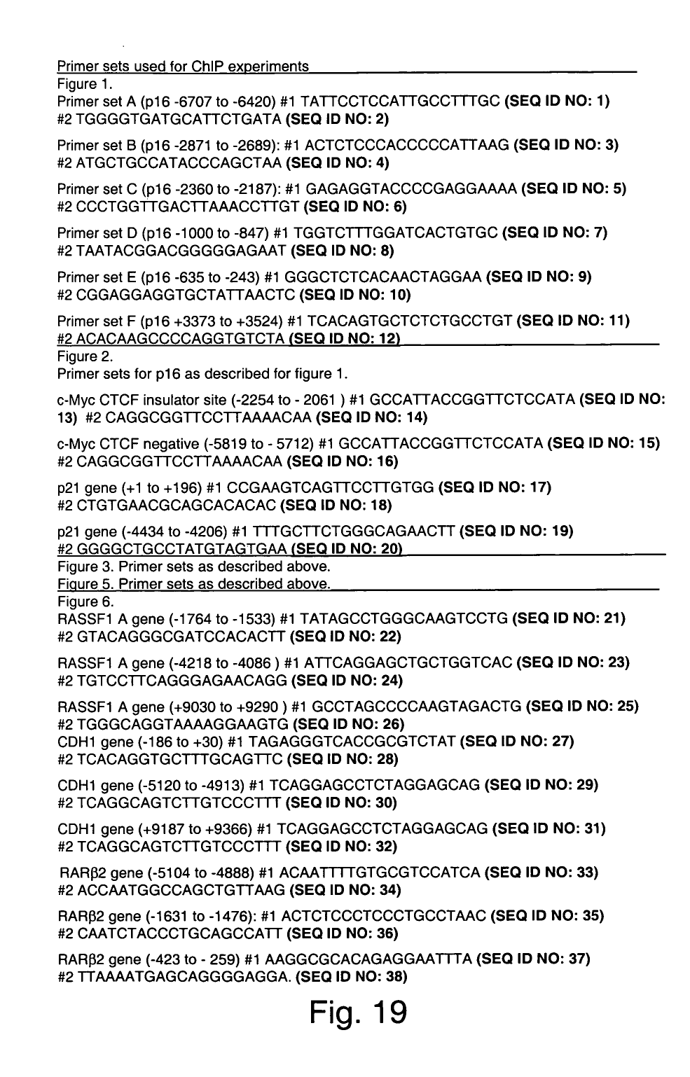

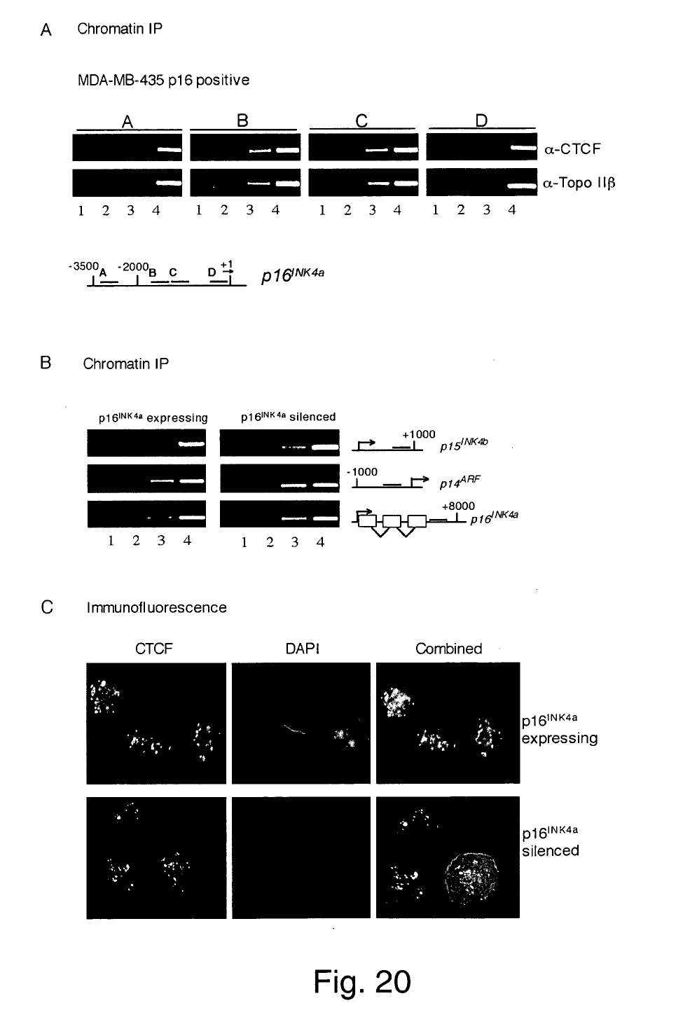

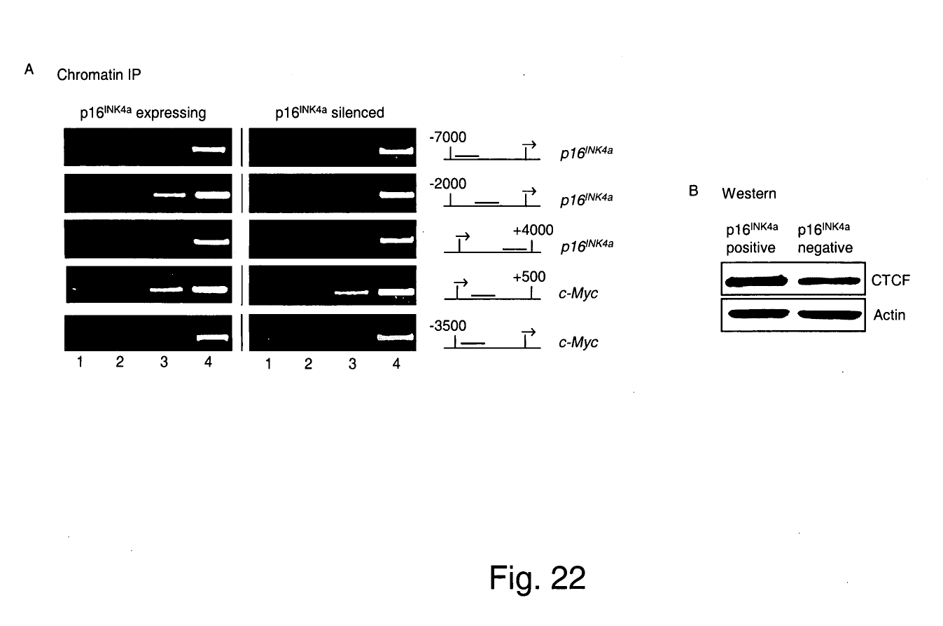

CTCF is a ubiquitous, multifunctional protein that has a critical role in organizing distinct chromosomal domains through boundary/insulator formation (Filippova et al. (2005) “Boundaries between chromosomal domains of X inactivation and escape bind CTCF and lack CpG methylation during early development.” Dev Cell 8: 31-42; Ishihara et al. (2006) “CTCF-dependent chromatin insulator is linked to epigenetic remodeling.” Mol Cell 23: 733-742; Splinter et al. (2006) “CTCF mediates long-range chromatin looping and local histone modification in the beta-globin locus.” Genes Dev 20: 2349-2354) and in repressing or activating transcription. A model of a CpG island-containing promoter in an active or silenced state is shown in FIG. 11. Because we saw a pronounced change in chromatin structure upstream of the p16 gene when active or silenced (FIG. 1), we explored the possibility that CTCF can associate within this region. To address this issue, a ChIP analysis was performed to identify sites of CTCF interaction within −7 to +4 kb of the p16 gene locus in expressing and non-expressing cells. FIG. 20 provides the results of experiments that were performed to analyze CTCF binding and cellular localization. Our data revealed that in p16-expressing cells, CTCF clearly binds downstream (amplicon D) of the region enriched for marks of heterochromatin within −2 kb and +1 of the active p16 gene (FIGS. 2A, 20A and 22A). Chromatin IP using anti-CTCF antibody localizes CTCF binding to a region approximately 1 kb upstream of the p16 start site. The CTCF binding partner Topo IIβ also binds this region. (Lanes are as follow: 1. H₂O control; 2. No antibody control; 3. IP using anti-CTCF antibody; 4. 1.6% total input DNA.) However, no CTCF binding was observed at other distal regions in the locus near −7 kb (amplicon A) or +4 kb (amplicon F). Surprisingly, when we examined cells containing a silenced p16 gene, CTCF interaction at the upstream promoter site was not apparent. Yet, we detected CTCF binding at a well-characterized target gene, c-Myc, in both p16-expressing and non-expressing cell types. This indicates that loss of CTCF binding from the p16 gene in T47D cells is not due to a general defect in the ability of the endogenous protein to associate with its chromosomal targets. Moreover, CTCF dissociation from the p16 gene is not mechanistically linked to the stability of other CTCF interactions that we examined in the INK4/ARF locus (FIG. 20B). FIG. 20B provides the results of ChIP experiments using CTCF specific antibody. Amplification of known CTCF sites demonstrates a different binding pattern in T47D cells, e.g., p16^{INK4a} silenced cells, and MDA-MB-435 cells, e.g., p16^{INK4a} expressing cells, at these loci than is observed at the p16^{INK4a} gene. (Lanes are as follow: 1. H₂O control; 2. No antibody control; 3. IP using anti-CTCF antibody; 4. 1.6% input.) No significant reduction in bulk CTCF protein levels (see western blot in FIG. 22B) or in cellular localization was observed between T47D and MDA-MB-435 cells (see FIG. 20C, which depicts CTCF staining using immunofluorescent antibodies in MDA-435 cells (top panels) and T47D cells (bottom panels)). Importantly, loss of CTCF interaction is not a consequence of cessation of p16 transcription since CTCF binding remains stable upon p16 gene inactivation by pharmacological inhibitors (FIG. 16). FIG. 16 shows the results of experiments that were performed to determine whether inhibition of transcription impacts CTCF binding. FIG. 16A shows the results of RT-PCR that was performed to confirm the inhibition of p16 and RASSF1A transcription in response to 24 hour treatments of MDA-MB-435 cells with Actinomycin D or Flavopiridol. FIG. 16B shows ChIP analyses of MDA-MB-435 and T47D cells treated with 2.5 μg/ml Actinomycin D or 1 μM Flavopiridol for 24 hours. CTCF was immunoprecipitated and analyzed for association with the p16^{INK4a} and RASSF1A gene. NA represents no antibody control.

CTCF is a predominately nuclear protein that is delocalized to the cytoplasm in some primary breast tumor samples (Butcher and Rodenhiser (2007) “Epigenetic inactivation of BRCA1 is associated with aberrant expression of CTCF and DNA methyltransferase (DNMT3B) in some sporadic breast tumours.” Eur J Cancer 43: 210-219). We examined the cellular localization of CTCF protein in MDA-MB-435 and T47D cells by immunofluorescent staining and found that CTCF is primarily nuclear with little cytoplasmic redistribution in either cell type (FIG. 20C). Mutation of CTCF can also occur as a rare event in some breast cancers (Zhou et al. (2004) “A screen for germline mutations in the gene encoding CCCTC-binding factor (CTCF) in familial non-BRCA1/BRCA2 breast cancer.” Breast Cancer Res 6: R187-190); however, our DNA sequencing analysis failed to identify any CTCF mutation in T47D cells.

Next, we monitored the stability of CTCF binding at other genes in T47D cells in addition to the c-Myc promoter. To this end, we examined CTCF interaction at three known recognition sites within the INK4/ARF locus. Previous work suggested the existence of CTCF sites at the p14 promoter (Filippova et al. (2002) “Tumor-associated zinc finger mutations in the CTCF transcription factor selectively alter tts DNA-binding specificity.” Cancer Res 62: 48-52), and two recent studies found novel CTCF sites near the p15 start site and at the 3′ flanking region of the last p16 exon (Barski et al. (2007) “High-resolution profiling of histone methylations in the human genome.” Cell 129: 823-837; Kim et al. (2007) “Analysis of the vertebrate insulator protein CTCF-binding sites in the human genome.” Cell 128: 1231-1245).

In the ChIP analysis shown in FIG. 20B, we observed CTCF binding at the p14 promoter in both MDA-MB-435 and T47D cells. However loss of CTCF from the p15 gene and potentially weak binding near the last p16 exon was apparent in MDA-MB-435 cells. Thus, the pattern of binding at these CTCF sites is distinct from the CTCF binding at the p16 upstream promoter (FIG. 1B). This supports the notion that when bound to the upstream promoter, CTCF plays a functionally distinct role in regulating p16 gene activity. Moreover, its dissociation from this particular region is not mechanistically linked to the stability of the other CTCF interactions that we examined in the INK4/ARF locus.

p16 Gene Expression Correlates with CTCF Binding Near its Chromosomal Boundary in Multiple Types of Human Cancer Cells

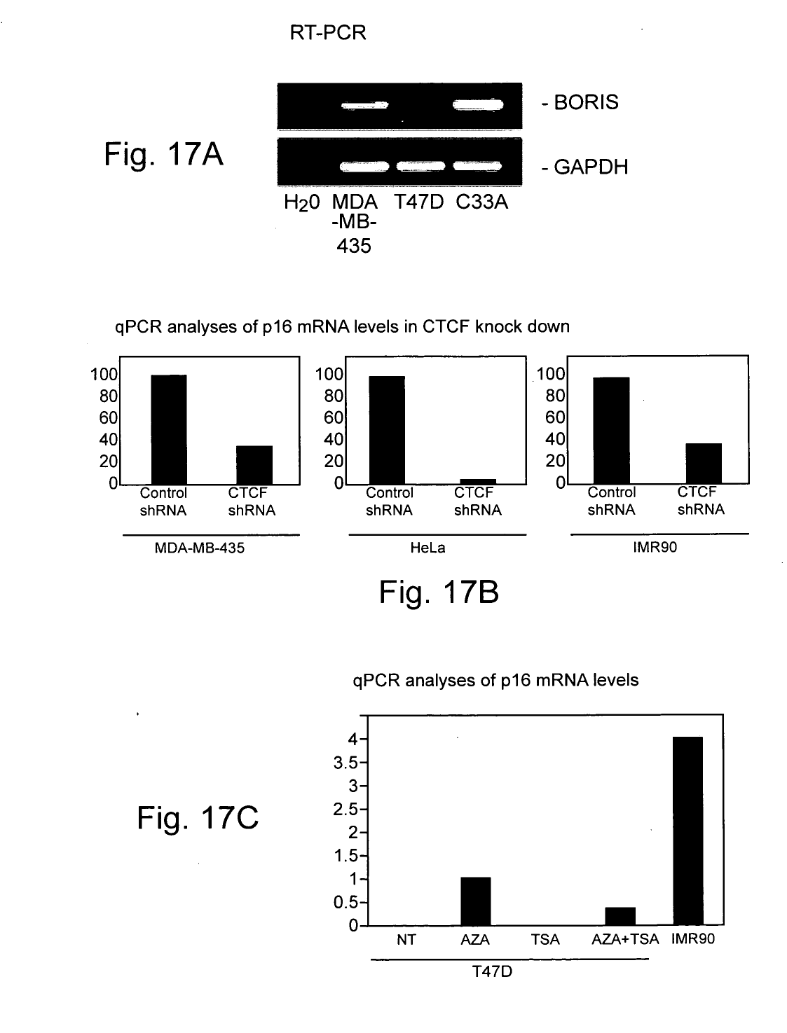

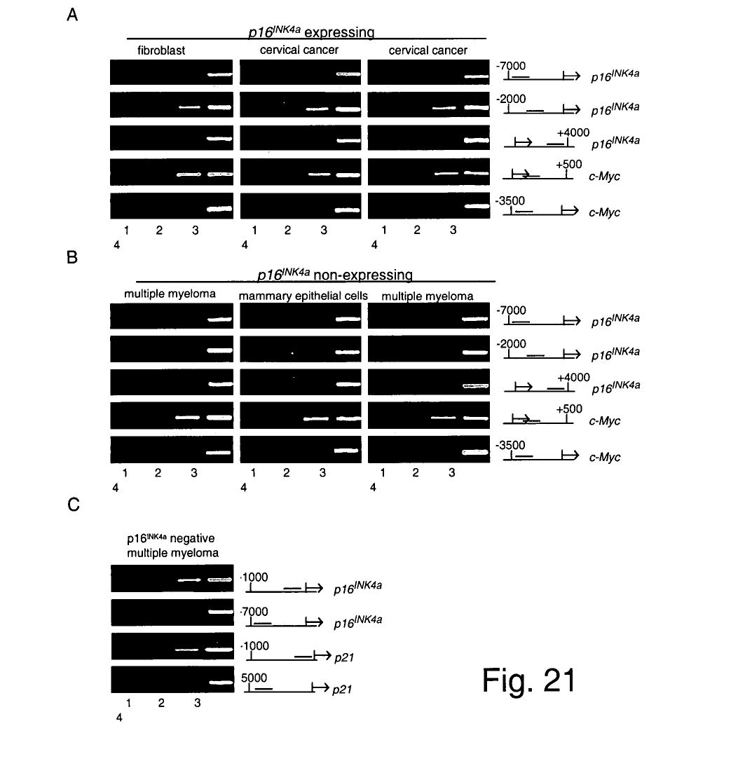

Having established a strong correlation between CTCF interaction with the p16 upstream promoter and p16 expression in breast cancer cell lines, we asked whether our observations could be extended to other types of human cancer cells. For example, the p16 gene is a frequent target of epigenetic inactivation in primary multiple myeloma cells (Ng et al. (1997) “Frequent hypermethylation of p16 and p15 genes in multiple myeloma.” Blood 89: 2500-2506). As shown in the ChIP analysis in (FIGS. 2B and 21A), CTCF binding is highly correlated with p16 expression in diverse cell types such as non-transformed fibroblasts (IMR90) and the cervical cancer cell lines (HeLa, C33A). Conversely, in two multiple myeloma cell lines (U266, KMS12) and a primary breast epithelial-derived cell line (vHMEC), each of which harbors a silenced p16 gene, CTCF binding was lost from the upstream promoter (FIG. 21B). Consistent with our findings in the MDA-MB-435 and TD47 breast cancer cell lines (FIG. 2A, Lanes: 1. H₂O; 2. no antibody; 3. anti-CTCF antibody; 4. 1.6% input DNA; and FIG. 22A), CTCF interaction at the c-Myc promoter was constant in all cell types examined (FIG. 2B, Lanes: 1. H₂O; 2. no antibody; 3. anti-CTCF antibody; 4. 1.6% input DNA; and FIGS. 21A and B). Thus, CTCF associates with the active, but not silent, p16 gene. Loss of CTCF binding from the p16 upstream promoter near its chromosomal boundary is correlated with transcriptional silencing in both human breast cancer and multiple myeloma cell lines even though CTCF interaction with c-Myc remains unaffected. The loss of CTCF binding at p16 could not be attributed to aberrant expression and recruitment of BORIS to replace CTCF, as we saw no correlation between p16 silencing and BORIS expression (FIG. 17A). FIG. 17 provides the results of experiments that were performed to quantify p16 mRNA levels by qPCR. In FIG. 17A, RT-PCR analysis of BORIS expression in human cancer cells shows that BORIS expression does not correlate with p16 silencing in T47D cells. qPCR analyses of p16 mRNA levels in CTCF knockdown cells in FIG. 17B show that all cell types studied show significant reduction of p16 transcripts. The most pronounced reduction was observed in HeLa cells. (mRNA levels were normalized to 18S mRNA.) qPCR analyses of p16 mRNA levels in T47D cells treated with AZA and trichostatin A (shown in FIG. 17C) show that cellular p16 levels were not restored to physiological levels in response to AZA as demonstrated by comparison to IMR90 p16 levels.

Next, we asked whether the striking relationship between CTCF binding and p16 gene transcription could be extended to other factors that are postulated to regulate p16 expression. To address this, we examined association of the ubiquitous nuclear factor Spl with the p16 promoter in both p16-expressing and non-expressing cell lines, because Spl has been implicated in p16 gene transactivation (Wu et al. (2007) “Sp1 is essential for p16 expression in human diploid fibroblasts during senescence.” PLoS ONE 2: e164). ChIP analyses of Sp1 binding to the p16 promoter were performed using multiple myeloma (U266) cells (FIG. 2C). p21 was used as a positive control. Unexpectedly, we found strong Sp1 binding to the p16 promoter in multiple myeloma cells (U266) where the gene is silent (FIG. 2C, Lanes: 1. H₂O; 2. no antibody; 3. anti-CTCF antibody; 4. 1.6% input DNA; FIG. 21C). The extent of Sp1 interaction with p16 was comparable to its association with the p21 gene (FIG. 21C), where Sp1 has previously been shown to interact (Biggs et al. (1996) “The role of the transcription factor Sp1 in regulating the expression of the WAF1/CIP1 gene in U937 leukemic cells.” J Biol Chem 271: 901-906). To extend this analysis, Sp1 binding to the p16 promoter was examined using real-time PCR. Quantitative analysis of Sp1 binding revealed that, unlike CTCF, there is no clear correlation between Sp1 interaction with the p16 promoter and its transcriptional activity (FIG. 2D, Lanes correspond to Sp1 IPs from the following cells: 1. cervical cancer (HeLa); 2. human primary fibroblasts (IMR90); 3. breast cancer (MDA-MB-435); 4. multiple myeloma (U266); 5. multiple myeloma (KMS12); 6. breast cancer (T47D); 7. primary breast epithelial-derived (vHIMEC)). From these data, we conclude that transcriptional silencing of the p16 promoter is not due to occlusion of binding to regulatory factors in general. Instead, silencing can result from the spread of heterochromatin caused by loss of the upstream chromatin boundary that is maintained by CTCF binding. Dissociation of another promoter-bound activator, Spl, does not have this effect.

CTCF Epigenetically Regulates the p16 Promoter and Gene Expression

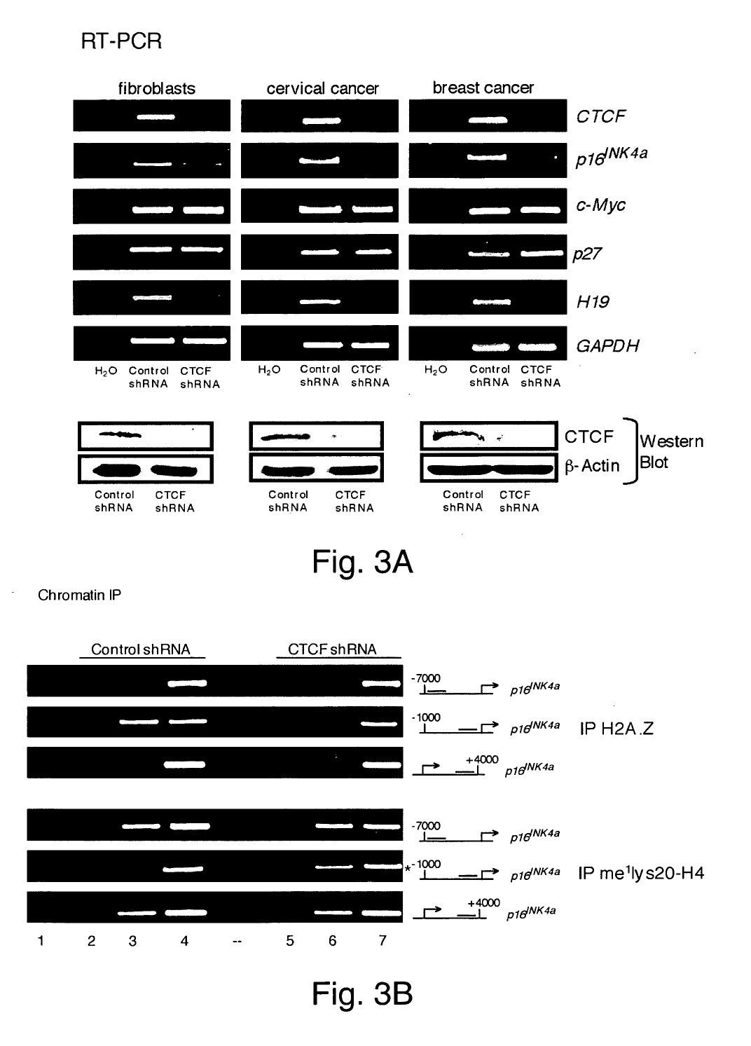

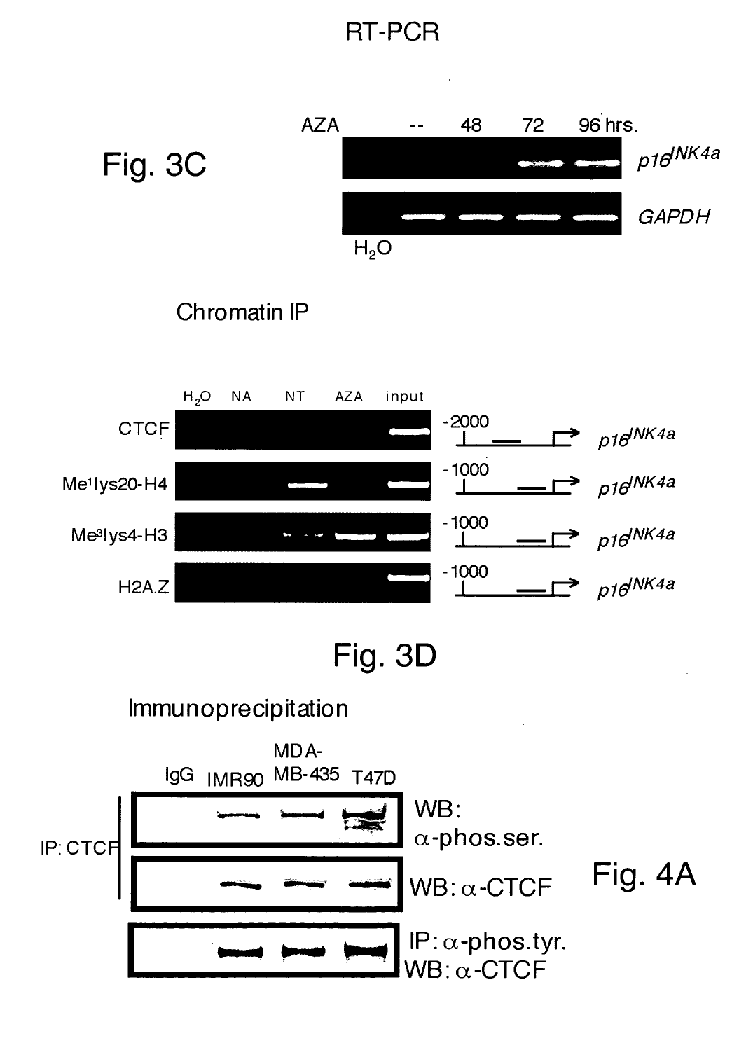

CTCF has previously been shown to have a myriad of nuclear functions including regulating insulator/boundary activity and repressing or activating transcription (Recillas-Targa et al. (2006) “Epigenetic boundaries of tumour suppressor gene promoters: the CTCF connection and its role in carcinogenesis” J Cell Mol Med 10: 54-568; Wallace and Felsenfeld (2007) “We gather together: insulators and genome organization.” Curr Opin Genet Dev 17: 400-407). To investigate the functional role of CTCF at the p16 upstream region, we used shRNA to decrease expression of CTCF in several cell lines that contained an active p16 gene. The results of CTCF knockdown in transcriptional silencing of the p16 gene are shown in FIG. 3. Reduction of CTCF in each cell line infected with either control (scrambled) or CTCF-specific shRNA was confirmed at the level of mRNA by RT-PCR (FIG. 3A, upper panels) and protein by Western analysis (FIG. 3A, lower panels). Near complete ablation of cellular CTCF resulted in considerably reduced p16 mRNA levels in fibroblasts (IMR90), cervical cancer cells (HeLa), and breast cancer cells (MDA-MB-435), whereas no effect on p16 expression was observed in cells infected with a scrambled shRNA (FIGS. 3A and 17B). In addition, mRNA abundance of the H19 gene was significantly decreased in each CTCF knockdown cell line, consistent with the demonstrated involvement of CTCF in H19 expression (Szabo et al. (2004) “Role of CTCF binding sites in the Igf2/H19 imprinting control region.” Mol Cell Biol 24: 4791-4800). Expression of the GAPDH gene, which served as a control for total mRNA abundance, was also unchanged by the absence of CTCF. In contrast to a previous report (Qi et al. (2003) “CTCF functions as a critical regulator of cell-cycle arrest and death after ligation of the B cell receptor on immature B cells.” Proc Natl Acad Sci USA 100: 633-638), we observed no change of the cell cycle inhibitor p27 transcript levels upon CTCF knockdown, which may reflect tissue-specific consequences of CTCF depletion. Reduction of p16 and H19 transcripts is therefore unlikely to be cell cycle-specific since mRNA levels of p27, were not affected. Unexpectedly, transcript levels of the CTCF target gene c-Myc remained impervious to loss of CTCF. This suggests that CTCF can have distinct functional roles at the p16, H19, and c-Myc genes with different requirements for continuous binding versus transient binding.

Recent reports have implicated CTCF as a regulator of epigenetic modifications in mammalian cells (Splinter et al. (2006) “CTCF mediates long-range chromatin looping and local histone modification in the beta-globin locus.” Genes Dev 20: 2349-2354; Zhao and Dean (2004) “An insulator blocks spreading of histone acetylation and interferes with RNA polymerase II transfer between an enhancer and gene.” Nucleic Acids Res 32: 4903-4919). Knockdown of the CTCF interacting partner CHD8 also causes changes in histone modifications at CTCF-bound sites (Ishihara et al. (2006) “CTCF-dependent chromatin insulator is linked to epigenetic remodeling.” Mol Cell 23: 733-742). To explore the possibility that CTCF plays a role in chromatin organization at the p16 locus, we analyzed histone modifications at the p16 promoter in breast cancer cells (MDA-MB-435) whose CTCF levels were ablated by shRNA treatment. ChIP analysis of the p16 gene were performed after CTCF knockdown in p16-expressing cells (MDA-MB-435) using antibodies to the histone variant H2A.Z and Me1H4K20 (FIG. 3B). Reactions from cells infected with scrambled shRNA (lanes 2-4) and CTCF-specific shRNA (lanes 5-7) are designated. (Lanes: 1. H2O; 2 no antibody; 3. anti-H2A.Z antibody; 4. 1.6% input DNA; 5. no antibody; 6. anti-Me1H4K20 antibody; 7. 1.6% input DNA; * denotes 0.25% input DNA in lanes 4 and 7 for the fifth panel (p16 proximal promoter) only.) Most strikingly, we observed a significant reduction of the histone variant H2A.Z at the p16 promoter upon loss of cellular CTCF (FIG. 3B, upper panels) and an increase in monomethylation of H4K20 in the same region (lower panels). The loss of H2A.Z and 3′ shift of the repressive histone mark to the region downstream of the −2 kb boundary corresponds to the epigenetic characteristics of the silenced p16 gene (FIG. 1D), which no longer interacts with CTCF (FIGS. 2A and 22A) and apparently undergoes heterochromatin “spreading” from upstream regions. Thus, our results are consistent with the idea that CTCF binding is required to maintain a chromosomal boundary near −2 kb, which preserves the p16 gene in a transcriptionally active chromatin domain.

Pharmacological Treatment of Cancer Cells Restores Temporary p16 Gene Transcription but not CTCF Binding

The p16 tumor suppressor gene is commonly silenced in numerous types of human cancers and remains a relevant therapeutic target of wide interest. One method that is extensively employed to restore p16 expression, both clinically and in vitro, is treatment of cancer cells with hypomethylating-nucleoside analogues such as 5′AZA-2′-deoxycytidine (AZA) (Otterson et al. (1995) “CDKN2 gene silencing in lung cancer by DNA hypermethylation and kinetics of p16INK4 protein induction by 5-aza 2′ deoxycytidine.” Oncogene 11: 1211-1216; Schrump et al. (2006) “Phase I study of decitabine-mediated gene expression in patients with cancers involving the lungs, esophagus, or pleura.” Clin Cancer Res 12: 5777-5785), which reverses DNA methylation. We reasoned that treatment of cells with AZA might also restore CTCF binding at the p16 upstream promoter through one of two mechanisms. First, CTCF is known to bind DNA in a methylation-sensitive fashion (Hark et al. (2000) “CTCF mediates methylation-sensitive enhancer-blocking activity at the H19/Igf2 locus.” Nature 405: 486-489); thus, demethylation of the p16 locus might allow CTCF to reassociate. Second, demethylation of target promoters by AZA can change the surrounding chromatin structure (Fahrner et al. (2002) “Dependence of histone modifications and gene expression on DNA hypermethylation in cancer.” Cancer Res 62: 7213-7218), which can facilitate rebinding of regulatory proteins, as observed for Spl (Zhang et al. (2005) “Insensitivity to transforming growth factor-beta results from promoter methylation of cognate receptors in human prostate cancer cells (LNCaP).” Mol Endocrinol 19: 2390-2399). We conducted a time course of p16 mRNA induction after AZA treatment of breast cancer cells (T47D), which contain a methylated and silenced p16 gene. The results of this experiment, which were analyzed via RT-PCR, are shown in FIG. 3C. Significant reactivation of p16 expression was observed by 72 hours (FIGS. 3C and 17C). No synergistic p16 gene reactivation was observed in T47D cells treated with both AZA and the HDAC inhibitor Trichostatin A (FIG. 17C).

To examine whether any changes in histone modifications or potential reassociation of CTCF had occurred after reversal of p16 transcriptional silencing, a ChIP analysis of the p16 promoter was performed in T47D cells at 96 hours post-AZA treatment using the antibodies indicated in FIG. 3D. As shown in FIG. 3D, several alterations in chromatin structure at the p16 promoter were apparent (NA=no antibody, NT=no AZA treatment, AZA=AZA treatment, input=1.6% of total DNA). Notably, monomethylation of H4K20 and trimethylation of H3K4 were reversed in accordance with gene activity. However, AZA treatment did not result in the recruitment of CTCF or H2A.Z to the reactivated p16 gene. We also did not observe Sp1 at the p16 promoter after AZA treatment. While AZA treatment can reactivate p16 transcription, it is clear that it does not entirely reverse alterations that occur during gene silencing. This is consistent with a recent study showing only partial reversal of the histone code to an active state at the human MILH1 promoter after AZA treatment (McGarvey et al. (2006) “Silenced tumor suppressor genes reactivated by DNA demethylation do not return to a fully euchromatic chromatin state.” Cancer Res 66: 3541-3549). In fact, the general inability to sustain long-term p16 gene expression after reversal of epigenetic silencing by AZA (Egger et al. (2007) “Inhibition of histone deacetylation does not block resilencing of p16 after 5-aza-2′-deoxycytidine treatment.” Cancer Res 67: 346-353) can, in part, be explained by failure to reestablish the upstream chromatin domain boundary by CTCF.

Absence of CTCF PARlation in p16—Silenced Cells

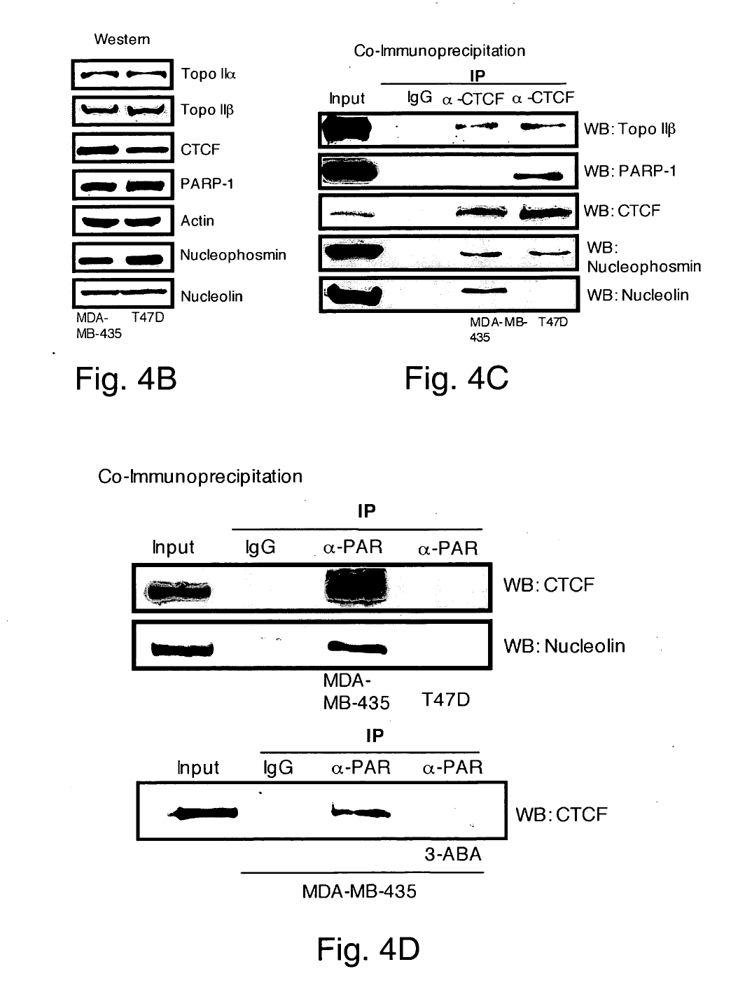

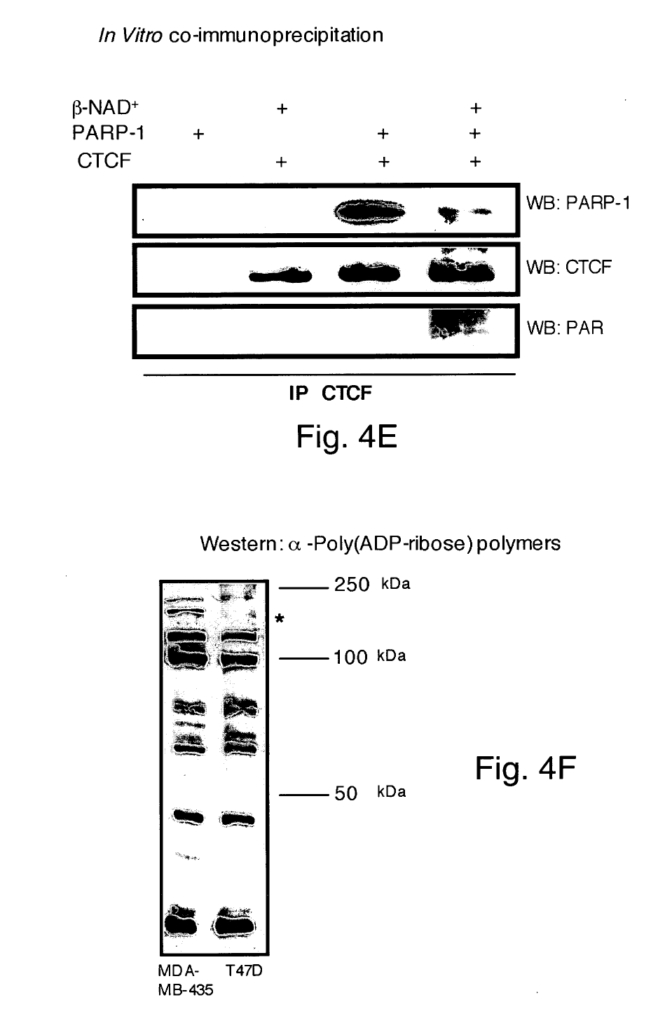

As described above, CTCF may be important for maintaining an active p16 gene. To further investigate possible CTCF defects that may impact its function in p16-silenced T47D breast cancer cells, we examined its post-translational modifications and association with several known protein interaction partners. CTCF is post-translationally modified by phosphorylation and poly(ADP-ribosyl)ation (PARlation) and interacts with multiple proteins, such as Toposiomerase IIα, Topoisomerase IIβ, Nucleolin, Nucleophosmin and PARP-1 (Yusufzai et al. (2004) “CTCF tethers an insulator to subnuclear sites, suggesting shared insulator mechanisms across species.” Mol Cell 13: 291-298). As shown in FIG. 4A, similar extents of CTCF phosphorylation at both serine (left panel) and tyrosine (right panel) residues were observed in normal fibroblasts, p 16-expressing MDA-MB-435 and non-expressing TD47 breast cancer cells as determined by immunoprecipitation. In addition, western blots were performed to determine the protein levels of CTCF and putative interacting partners in MDA-MB-435 and T47D cells (FIG. 4B). Cellular levels of CTCF and several known interaction partners, Topo IIα, Topo IIβ, Nucleophosmin and PARP-1, were comparable in MDA-MB-435 and TD47 cells as well as a new interactor, Nucleolin (FIG. 4B). Co-immunoprecipitation of CTCF complexes indicated that CTCF interacts with Topo HP and Nucleophosmin similarly in both cell types but has opposite interaction characteristics with PARP-1 which, surprisingly, only associates with CTCF in p16 silenced cells (FIG. 4C). Moreover, PARP-1 and Nucleolin appear to associate with CTCF in a mutually exclusive manner, with Nucleolin being present in CTCF complexes only in p16 expressing cells. To explore this further, we examined the PARlation status of CTCF in each cell type using an antibody that recognizes ADP-ribose polymers (PAR). Unexpectedly, we found PARlation associated with both CTCF and Nucleolin in p16-expressing MDA-MB-435 cells but not in p16-silenced TD47 cells (FIG. 4D, upper panels). This PARlation is inhibited upon addition of the PARlation inhibitor 3-Aminobenzamide (3-ABA) demonstrating the specificity of this reaction (FIG. 4D, lower panel). The lower panel of FIG. 4D shows similar IP using material treated with 3-ABA for 24 hours. Inputs are equal to 2.5% starting material.

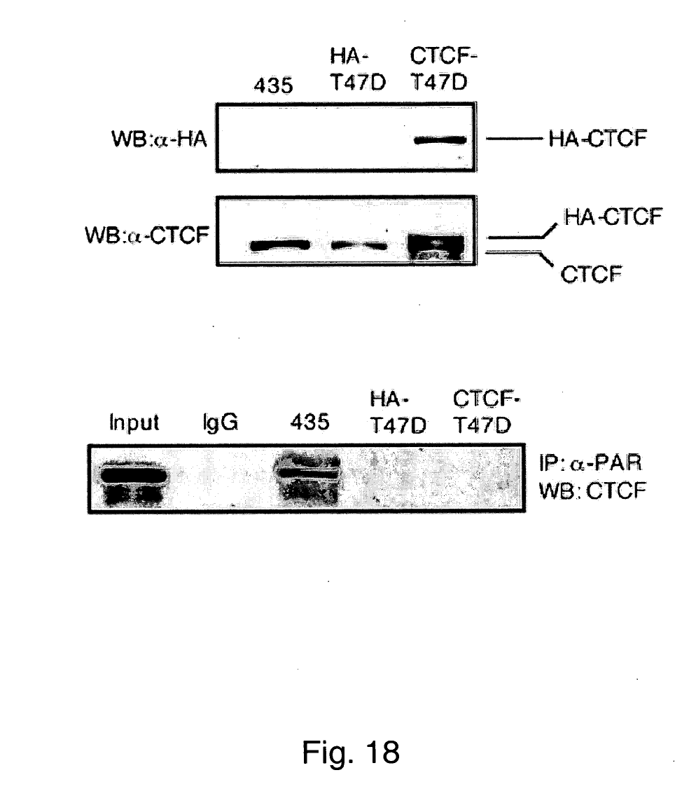

Initially, it appeared counterintuitive that CTCF could associate with PARP-1 but be unPARlated in TD47 cells, whereas in MDA-MB-435 cells the opposite was true: CTCF was PARlated and dissociated from PARP-1. We surmised that differences in CTCF-PARP-1 interaction dynamics might reflect defects in the poly(ADP)ribosylation enzymatic pathway in TD47 cells. To substantiate this, we performed an in vitro binding assay using recombinant PARP-1 and CTCF proteins with or without the obligate poly(ADP)ribosylation substrate, β-NAD⁺, under enzymatic reaction conditions. Immunoprecipitations of CTCF from the reactions following protein binding assays are shown in FIG. 4E. Interestingly, we found that in the absence of β-NAD⁺ a stable complex formed between CTCF and PARP-1 when co-precipitated. Yet when β-NAD⁺ was present and CTCF PARlated, the CTCF/PARP-1 complex dissociated (FIG. 4E). This supports the notion that upon CTCF PARlation, it dissociates from the PARP-1 enzyme as is observed in MDA-MB-435 cells. If the enzymatic reaction is not productive and CTCF remains unPARlated, the enzyme-substrate complex fails to release as seen in T47D cells (FIGS. 4C, 4D). An examination, e.g., via western blot, of total cellular proteins that are PARlated in either MDA-MB-435 or T47D extracts revealed a very similar pattern with the primary exceptions being two proteins in the size range of CTCF and larger whose modification is clearly impaired in T47D cells (FIG. 4F, asterisk denotes protein with similar molecular weight as CTCF that is differentially PARlated in MDA-MB-435 and T47D cells). This indicates that while there are no apparent gross defects in the poly(ADP)ribosylation enzymatic machinery, its ability to react with specific protein substrates, such as CTCF, is deregulated. Further supporting this hypothesis is our finding that reintroduction of exogenous CTCF into T47D cells does not reestablish CTCF PARlation (FIG. 18), possibly reflecting altered dynamics between PARP-1 Nucleolin, and Nucleophosmin. FIG. 18 provides the results of experiments that were performed to analyze the expression and PARlation of full-length recombinant CTCF in T47D cells. CTCF was introduced using a lentiviral delivery system (described below). Immunoprecipitations were done using an anti-PAR antibody on control cells infected with an empty vector as well as on CTCF-expressing cells. MDA-MB-435 cells were used as a positive control. Additional experiments show that the absence of CTCF PARlation results from defects in the addition of PAR residues by PAR polymerases rather than their deregulated turnover by poly(ADP-ribose)glycohydrolases (PARGs) (Klenova and Ohlsson (2005) “Poly(ADP-ribosyl)ation and epigenetics. Is CTCF PARt of the plot?” Cell Cycle 4:96-101).

Differential Patterns of PARlation and CTCF Partner Binding at the p16 Gene

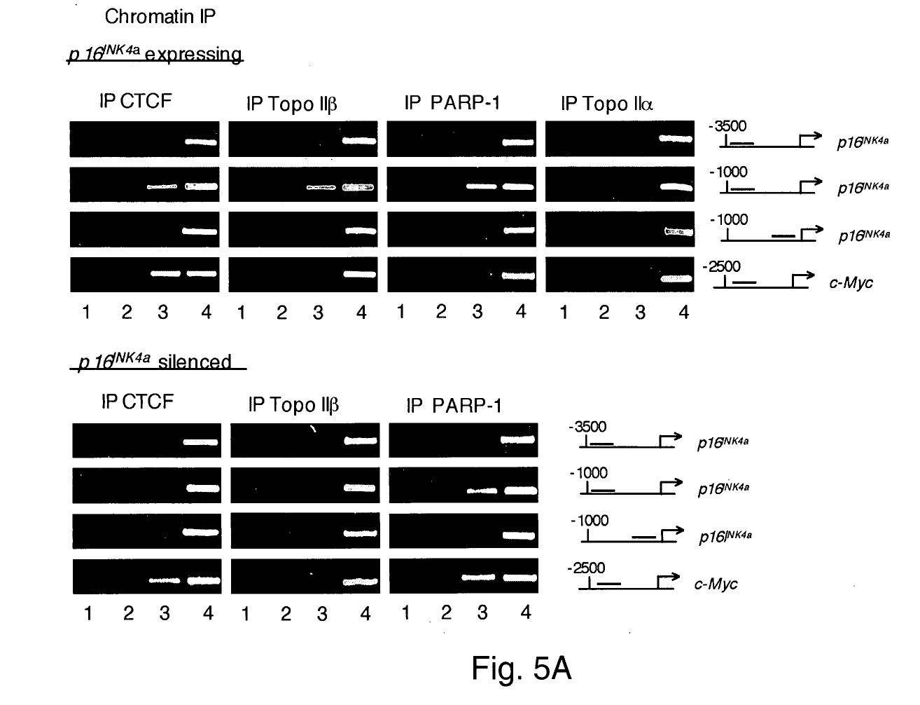

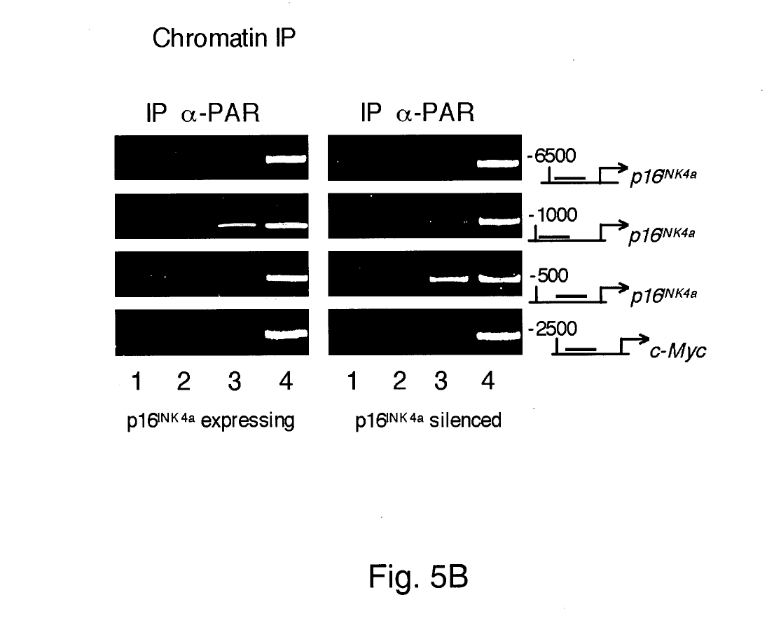

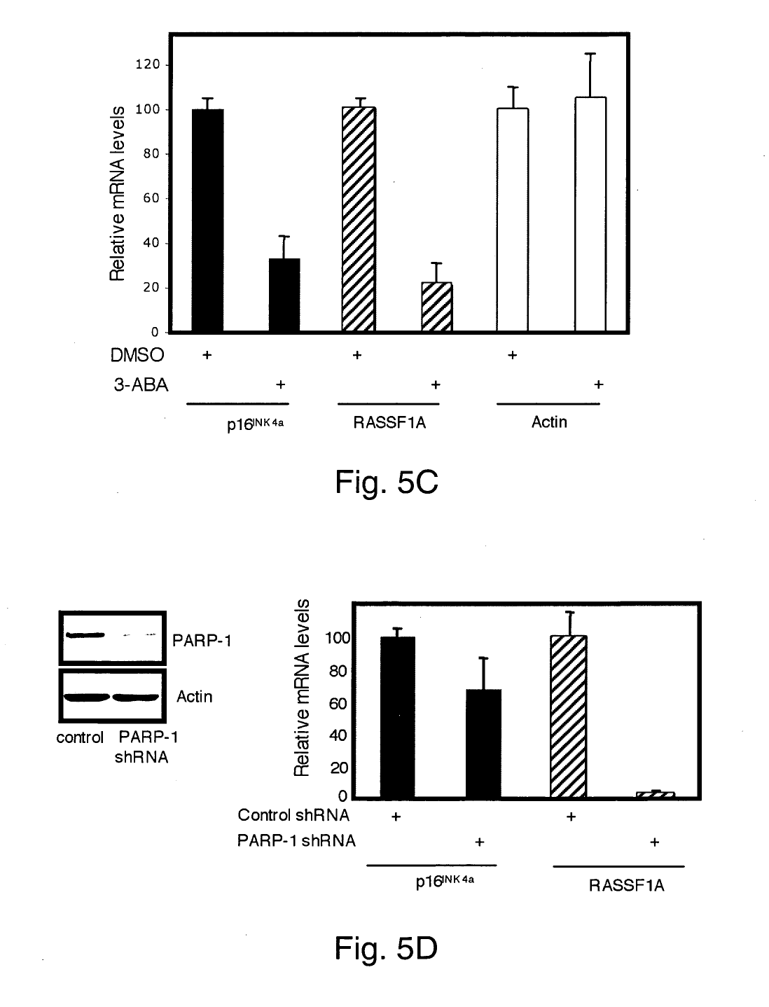

To determine whether known interacting partners of CTCF associate with the p16 gene when active or epigenetically silenced, we performed ChIP analyses on MDA-MB-435 or T47D extracts using antibodies to Topo Ha, Topo HO, and PARP-1. The pattern of PARlation at the p16 promoter region changes in p16-silenced cells. As shown in FIG. 5A, in p16-positive cells CTCF, Topo Hp, and PARP-1 each bind to the p16 gene in the region around −1 kb whereas no Topo Ha was detected in the distal or proximal promoter. At the CTCF binding site upstream of the c-Myc promoter, weak PARP-1 binding was observed but no association of Topo Ha or Topo H. (Lanes: 1. H₂O; 2. no antibody; 3. anti-H2A.Z antibody; 4. 1.6% input DNA.) By contrast, in p16-negative cells not only is CTCF lost from the silent p16 gene but Topo Hp is also dissociated. Interestingly, PARP-1 remains bound to the inactive p16 gene, apparently interacting independently of CTCF. PARP-1 binding to the CTCF site proximal to the c-Myc gene is also highly enriched in T47D cells. The presence of PARP-1 at the p16 and c-Myc genes led us to examine the distribution of chromatin-bound PARlated proteins using an anti-PAR antibody. ChIP analyses of PARlation pattern at the p16 and c-Myc genes were performed using the same cells as in FIG. 5A. The results of these analyses are provided in FIG. 5B. (Lane order is as in FIG. 5A, but showing amplification of 0.25% input material.) As shown in FIG. 5B, PARlation is enriched at the −1 kb region of the expressed p16 gene, (possibly indicating the presence of a PARlated CTCF), with low level PARlation within the proximal promoter. However, when the p16 gene is silenced upon loss of CTCF, the pattern of PARlation shifts from −1 kb to being highly enriched at the proximal promoter, even though PARP-1 is still bound near −1 kb. This redistribution may reflect PARlation of heterochromatin components that are enriched after CTCF dissociates, such as histone H1. Notably, modification of these components is unaffected by the aberration in the pathway that prevents poly(ADP)ribosylation of CTCF, underscoring the specific nature of this defect. In contrast to the p16 locus, no PARlation was observed at the c-Myc insulator site even in the vicinity of bound CTCF and PARP-1. These results indicate that separate CTCF binding sites are distinct from one another in terms of cofactor interactions and PARlation, potentially allowing CTCF to exert specialized regulatory functions on different target genes.

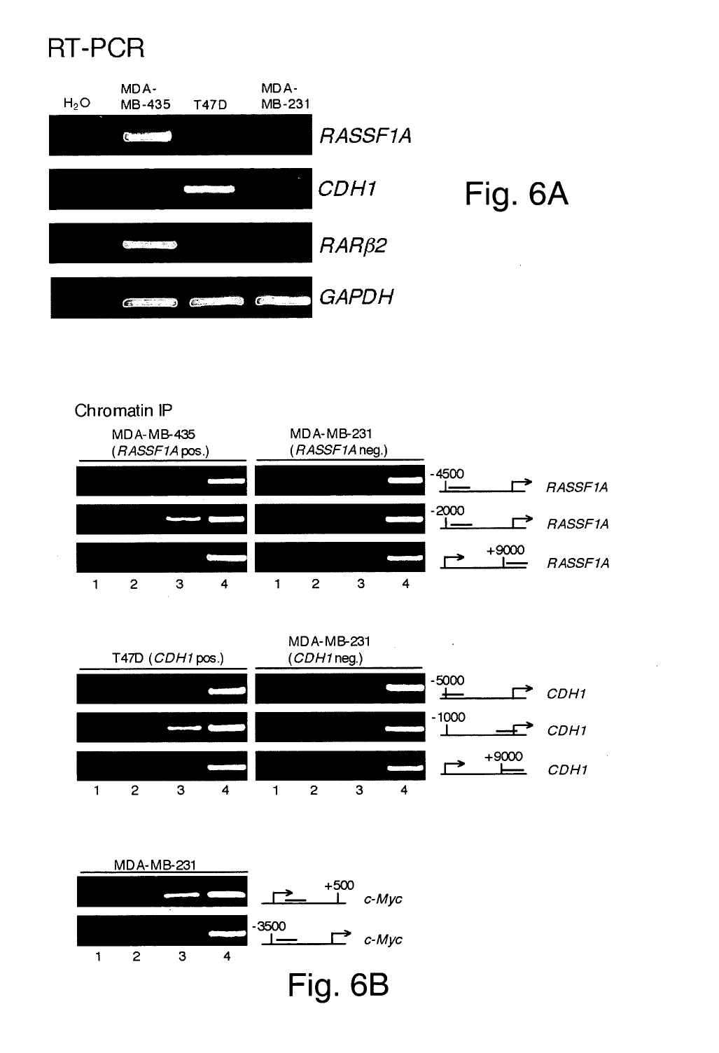

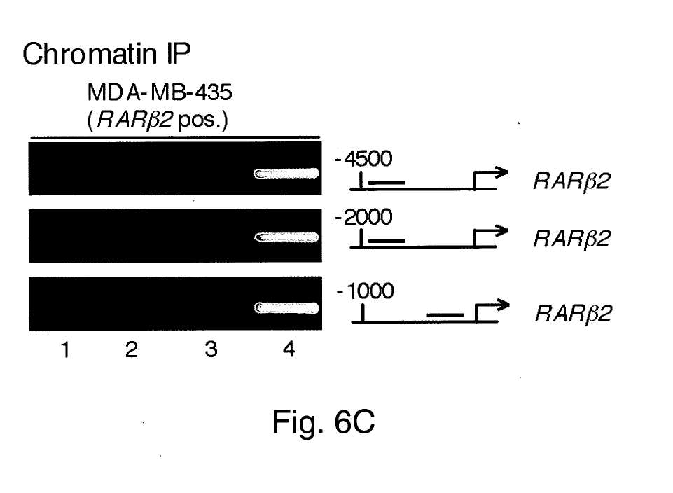

Next, we examined whether PARlation of target proteins impacts the expression of CTCF-regulated tumor suppressor genes. To achieve this, we perturbed cellular PARlation activity in p16-expressing cells by two approaches: first, incubation with the broad-spectrum PARP inhibitor 3-ABA; and second, shRNA-mediated ablation of PARP-1. The results are provided in FIGS. 5C and 5D, which show qPCR expression analysis of the CTCF target genes p16 and RASSF1A upon inhibition of PARP activity. Results are normalized to c-Myc levels. Error bars represent ±standard deviation. Amplification of mRNA from MDA-MB-435 cells treated for 24 hours with 5 mM 3-ABA is shown in FIG. 5C. Amplification of mRNA from MDA-MB-435 cells infected with shRNA directed towards PARP-1 is shown in FIG. 5D. In both cases, we observed a significant reduction of p16 mRNA levels as well as a dramatic decrease of the new CTCF target gene RASSF1A (FIGS. 5C, 5D, and 6A). Collectively, these data show that normal PARP activity is required for full activation of these CTCF target genes and that a disruption of this pathway can play a role in the long-term silencing of these tumor suppressors.

Epigenetic Silencing of the Tumor Suppressor Genes RASSF1a and CDH1 also Correlates with Loss of CTCF Binding

Having established that CTCF interaction upstream of the p16 promoter is abolished in several different types of human cancer cell lines in which the gene is hypermethylated and silenced, we speculated that CTCF binding sites might be present at other genes commonly silenced in cancer. To this end, we identified potential CTCF recognition sequences in the promoters of the RASSF1A, CDH1 and RAR/32 genes and analyzed these regions for expression, e.g., via RT-PCR, (FIG. 6A) and CTCF binding, e.g., via ChIP analysis, in several breast cancer cell lines. Similar to p16, the RASSF1a protein is a tumor suppressor (Dammann et al. (2000) “Epigenetic inactivation of a RAS association domain family protein from the lung tumour suppressor locus 3p21.3.” Nat Genet. 25: 315-319), and aberrant methylation of its gene is postulated to represent an early event in breast tumorigenesis (Strunnikova et al. (2005) “Chromatin inactivation precedes de novo DNA methylation during the progressive epigenetic silencing of the RASSF1A promoter.” Mol Cell Biol 25: 3923-3933; Yan et al. (2006) “Mapping geographic zones of cancer risk with epigenetic biomarkers in normal breast tissue.” Clin Cancer Res 12: 6626-6636). As shown in FIG. 6B (upper panels), a ChIP analysis of RASSF1A-positive cells (MDA-MB-435) clearly demonstrates recruitment of CTCF to a region upstream (−1.8 kb) of the promoter. However, in breast cancer cells (MDA-MB-231) in which RASSF1A is silenced and methylated, no such binding of CTCF was detected. (In FIG. 6B, Lanes: 1. H2O; 2. no antibody; 3. anti-CTCF antibody; 4. 1.6% input DNA.)

Hypermethylation of CDH1 in breast cancer results in a loss of E-cadherin expression (Graff et al. (1995) “E-cadherin expression is silenced by DNA hypermethylation in human breast and prostate carcinomas.” Cancer Res 55: 5195-5199) and is highly associated with an invasive and infiltrating phenotype (Shinozaki et al. (2005) “Distinct hypermethylation profile of primary breast cancer is associated with sentinel lymph node metastasis.” Clin Cancer Res 11: 2156-2162). CDH1-positive T47D breast cancer cells were examined to determine whether CTCF was bound to the CDH1 promoter when transcriptionally active. Again, we found CTCF binding at the immediate upstream (˜200 bp) region of a gene commonly methylated in cancer (FIG. 6B, middle panels). As with the p16 and RASSF1A genes, CTCF binding was not detectable in MDA-MB-231 cells where CDH1 is hypermethylated (FIG. 6B, middle panels). Interestingly, although CTCF is absent from the RASSF1A and CDH1 promoters in MDA-MB-231 cells, it is still bound to the c-Myc site (FIG. 6B, lower panels). This is consistent with our observations in other cell lines and indicates that CTCF in these cells is still functional to bind a subset of its target promoters even if it can no longer interact with specific tumor suppressor genes.

The RAR/32 gene is another common target of hypermethylation in breast cancer (Bovenzi et al. (1999) “DNA methylation of retinoic acid receptor beta in breast cancer and possible therapeutic role of 5-aza-2′-deoxycytidine.” Anticancer Drugs 10: 471-476). Clinical evidence has shown that hypermethylation of this gene, along with RASSF1A, can be a useful marker of increased breast cancer risk (Lewis et al. (2005). “Promoter hypermethylation in benign breast epithelium in relation to predicted breast cancer risk.” Clin Cancer Res 11: 166-172). However, upon examination of RARβ2-positive MDA-MB-435 breast cancer cells by ChM, we were unable to find CTCF association within the RARβ2 promoter or regions upstream (FIG. 6C, Lanes: 1. H2O; 2. no antibody; 3. anti-CTCF antibody; 4. 1.6% input DNA). From these data we conclude that loss of CTCF binding from critical sites is a feature common to several genes that are frequently silenced in human cancers; however, this correlation does not apparently exist for all targets of aberrant hypermethylation.

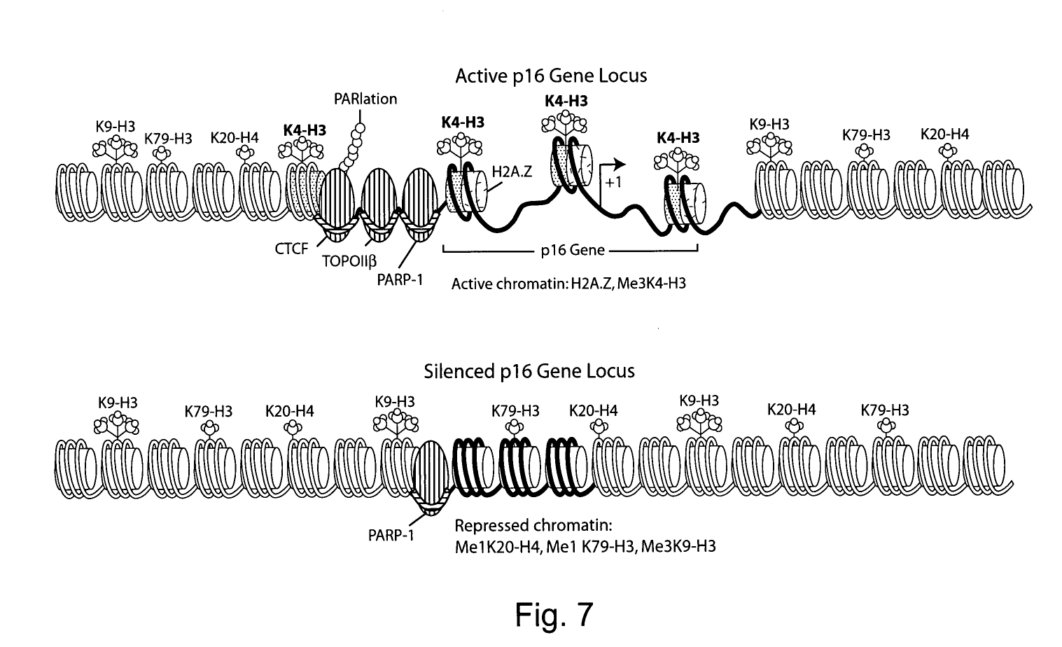

Our studies reveal a novel epigenetic mechanism of p16 transcriptional control that is deregulated when the gene is aberrantly silenced in human cancer cells. A model of CTCF function in aberrant tumor suppressor gene silencing in human cancers is schematically depicted in FIG. 7. We observed that the chromatin structure surrounding the active p16 gene is highly organized with a discrete partition of histone modifications at approximately 2 kb upstream of the transcription start site. We were surprised to find highly enriched marks of repressed chromatin, including monomethylated-H4K20, dimethylated-H3K27, trimethylated-H3K9, and monomethylated-H3K79, so close to a transcriptionally active gene. As a biological output, H3K79 methylation is primarily associated with transcriptional elongation (Krogan et al. (2003) “The Pafl complex is required for histone H3 methylation by COMPASS and Dotlp: linking transcriptional elongation to histone methylation.” Mol Cell 11: 721-729). The enrichment of this mark upstream of p16 can be a reflection of transcription through the adjacent p14 gene or general disorganization of chromatin 5′ of the −2 kb boundary. As expected, high levels of trimethylated-H3K4 are present surrounding the active p16 promoter plus a significant enrichment of the histone variant H2A.Z. In mammalian cells the role of H2A.Z is somewhat controversial. H2A.Z is implicated as a stabilizer of heterochromatin (Rangasamy et al. (2004) “RNA interference demonstrates a novel role for H2A.Z in chromosome segregation.” Nat Struct Mol Biol 11: 650-655), but is present in promoters of genes poised for activation (Farris et al. (2005) “Transcription-induced chromatin remodeling at the c-myc gene involves the local exchange of histone H2A.Z.” J Biol Chem 280: 25298-25303; Gevry et al. (2007) “p21 transcription is regulated by differential localization of histone H2A.Z.” Genes Dev 21: 1869-1881), and can contribute to nucleosome instability (Jin and Felsenfeld (2007) “Nucleosome stability mediated by histone variants H3.3 and H2A.Z.” Genes Dev 21: 1519-1529). Our data support a role for H2A.Z in poising the p16 gene for activation. Future studies can further illuminate the role of H2A.Z positioning at the p16 promoter in response to activation signals during cell senescence.

In p16-silenced breast cancer cells, partitioning of the p16 upstream region into distinct chromatin domains is lost and accompanied by disappearance of H2A.Z and trimethylated-H3K4 within 2 kb of the inactivated promoter. This demonstrates that deregulation of epigenetic processes at silenced promoters is not limited to DNA methylation and histone modification but can include placement of variant histones like H2A.Z. Upon loss of the −2 kb boundary, the repressive histone marks trimethylated-H3K9, monomethylated-H4K20, and monomethylated-H3K79 spread throughout the entire p16 promoter region. A widespread decrease in trimethylated-H4K20 is characteristic of cancer cells (Fraga et al. (2005) “Loss of acetylation at Lys16 and trimethylation at Lys20 of histone H4 is a common hallmark of human cancer.” Nat Genet. 37: 391-400), but levels of monomethylated-H4K20 have not been extensively examined. It is possible that H4K20 switches from the “tri” to “mono” methyl form in cancer cells. The observed increase in methylated-H3K79 is consistent with previous reports that showed upregulation of this modification in cancer (Okada et al. (2005) “hDOT1L links histone methylation to leukemogenesis.” Cell 121: 167-178). To date, however, enhanced H3K79-methylation has been associated with aberrantly activated genes in leukemia. Further work should clarify whether these differences are tissue-specific. Overall, our analyses indicate that a chromatin boundary exists upstream of the p16 gene, which chromatin boundary is destabilized in certain human cancer cells leading to aberrant transcriptional inactivation.

CTCF is a multifunctional protein that has previously been associated with establishing transitions between distinct chromatin domains (Bell et al. (1999) “The protein CTCF is required for the enhancer blocking activity of vertebrate insulators.” Cell 98: 387-396; Filippova et al. (2005) “Boundaries between chromosomal domains of X inactivation and escape bind CTCF and lack CpG methylation during early development.” Dev Cell 8: 31-42) and with acting as a shield against the spread of heterochromatin (Cho et al. (2005) “Antisense transcription and heterochromatin at the DM1 CTG repeats are constrained by CTCF.” Mol Cell 20: 483-489). These studies, coupled with the detection of a chromatin boundary in the p16 upstream region led us to look for CTCF binding in the p16 promoter. Our analyses demonstrated CTCF interaction with this region that is strongly correlated with p16 transcription in a variety of human cell types. This data is consistent with recently published genome-wide screens that also identified sites of CTCF binding within the p16 promoter (Barski et al. (2007) “High-resolution profiling of histone methylations in the human genome.” Cell 129: 823-837; Kim et al. (2007) “Analysis of the vertebrate insulator protein CTCF-binding sites in the human genome.” Cell 128: 1231-1245). shRNA-knockdown studies revealed that CTCF plays an active role in maintaining p16 gene expression when associated near the upstream boundary perhaps through stabilization of chromatin in this region. A dramatic loss of H2A.Z and gain of monomethylated-H4K20 upon depletion of CTCF emphasizes an integral epigenetic organizational function for CTCF at this locus. It also suggests that CTCF facilitates the stabilization or deposition of this histone variant. Considering that both CTCF and H2A.Z are posited to play important structural roles (Rangasamy et al. (2004) “RNA interference demonstrates a novel role for H2A.Z in chromosome segregation.” Nat Struct Mol Biol 11: 650-655; Yusufzai et al. (2004) “CTCF tethers an insulator to subnuclear sites, suggesting shared insulator mechanisms across species.” Mol Cell 13: 291-298), we speculate that these two proteins can act cooperatively to organize nuclear chromatin in a spatial manner.

Intriguingly, we observed that CTCF is absent from the p16 upstream region in multiple types of human cancer cells where the p16 gene is silenced and methylated. We extended this finding to two other genes that are commonly silenced in cancer, RASSF1a and CDH1 (E-cadherin). Together, this indicates that loss of CTCF binding to critical regions can be a common event in epigenetic silencing of cancer-related genes. Indeed, CTCF has been suggested to regulate other genes such as BRCA1 (Butcher et al. (2004) “DNA binding sites for putative methylation boundaries in the unmethylated region of the BRCA1 promoter.” Int J Cancer 111: 669-678) and Rb (De La Rosa-Velazquez et. al. (2007) “Epigenetic regulation of the human retinoblastoma tumor suppressor gene promoter by CTCF.” Cancer Res 67: 2577-2585) by protecting them against DNA methylation. Our studies directly link loss of CTCF from endogenous tumor suppressor genes with their epigenetic silencing. Because CTCF is involved in blocking the spread of heterochromatin and directing interactions between chromosomes (Ling et al. (2006) “CTCF mediates interchromosomal co-localization between Igf2/H19 and Wsbl/Nfl.” Science 312: 269-272), its dissociation from these tumor suppressors can have multiple consequences that are detrimental to transcription and localized genomic stability. Furthermore, silencing of the p 16 gene is an early step in breast carcinogenesis (Foster et al. (1998) “Inactivation of p16 in human mammary epithelial cells by CpG island methylation.” Mol Cell Biol 18: 1793-1801), which can lead to subsequent genomic instability (McDermott et al. (2006) “p16(INK4a) prevents centrosome dysfunction and genomic instability in primary cells.”PLoS Biol 4: e51) and downstream methylation events (Reynolds et al. (2006) “Tumor suppressor p16INK4A regulates polycomb-mediated DNA hypermethylation in human mammary epithelial cells.” J Biol Chem 281: 24790-24802). CTCF can be considered a potentially critical target in tumor progression.

Surprisingly, we found that the probable cause of impaired CTCF binding to the p16 upstream region is defective poly(ADP-ribosyl)ation, resulting in the absence of CTCF PARlation in p16-silenced cells. Poly(ADP-ribosyl)ation has been shown to regulate multiple biological processes including DNA methylation, DNA repair, genotoxic stress, and epigenetic programming by post-translational modification of critical regulatory proteins and chromatin components (Kraus (2008) “Transcriptional control of PARP-1: chromatin modulation, enhancer-binding, coregulation, and insulation.” Curr Opin Cell Biol 20: 294-302). In fact, inhibition of CTCF PARlation is correlated with failure to maintain IGF2 gene imprinting and insulator function in general (Yu et al. (2004) “Poly(ADP-ribosyl)ation regulates CTCF-dependent chromatin insulation.” Nat Genet. 36: 1105-1110).

We find that in p16-expressing cells, PARlated CTCF dissociates from PARP-1 and is complexed with cofactors Topo HP, Nucleophosmin, and a new interactor, Nucleolin. Nucleolin is a multifunctional protein with roles in cell membrane signaling, ribosomal RNA processing within the nucleolus, chromatin remodeling and transcription (Mongelard and Bouvet (2007) “Nucleolin: a multiFACeTed protein.” Trends Cell Biol 17: 80-86). The functional connection between PARP-1, Nucleolin and Nucleophosmin is very intriguing. These proteins have been isolated as a complex, and PARP-1 and Nucleolin have been shown to organize genomic DNA into topologically distinct domains through interaction with matrix/scaffold attachment regions that anchor chromatin onto the nuclear matrix (Galande (2002) “Chromatin (dis)organization and cancer: BUR-binding proteins as biomarkers for cancer.” Curr Cancer Drug Targets 2: 157-190). Strikingly, unPARlated CTCF fails to release from PARP-1 and loses its association with Nucleolin, but not Topo IIβ or Nucleophosmin. Such a complex is apparently insufficient to generate the p16 gene boundary even in the presence of chromatin-bound PARP-1. This is supported by previous work that underscored the importance of PARlation for proper CTCF insulator function (Yu et al. (2004) “Poly(ADP-ribosyl)ation regulates CTCF-dependent chromatin insulation.” Nat Genet. 36: 1105-1110). Our data also reveal that functionally distinct CTCF complexes associate with the p16 and c-Myc genes that differ in the requirement for specific cofactors. These include PARP-1 and Topo IV, which can coregulate transcription of some genes through transient DNA breakage and repair mechanisms (Ju et al. (2006) “A topoisomerase II beta-mediated dsDNA break required for regulated transcription.” Science 312: 1798-1802). Thus, the absence of CTCF binding to p16, or other epigenetically silenced genes may result from defects in specific post-translational modifications, such as PARlation, or cofactor interactions without affecting the majority of CTCF genomic functions.

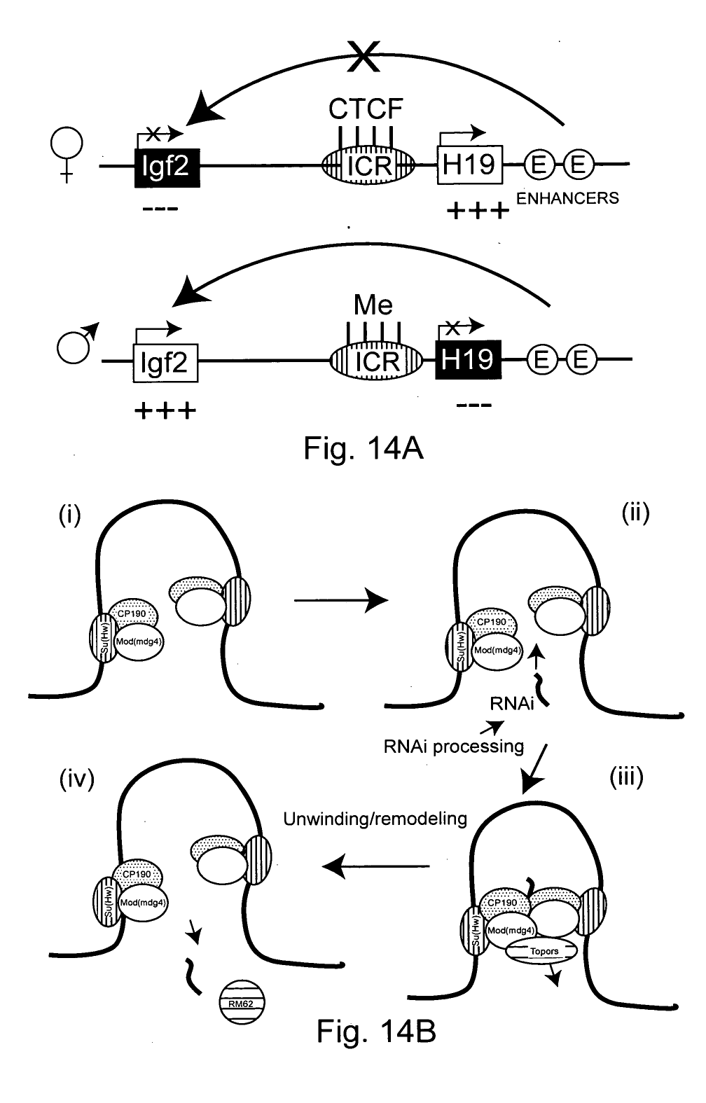

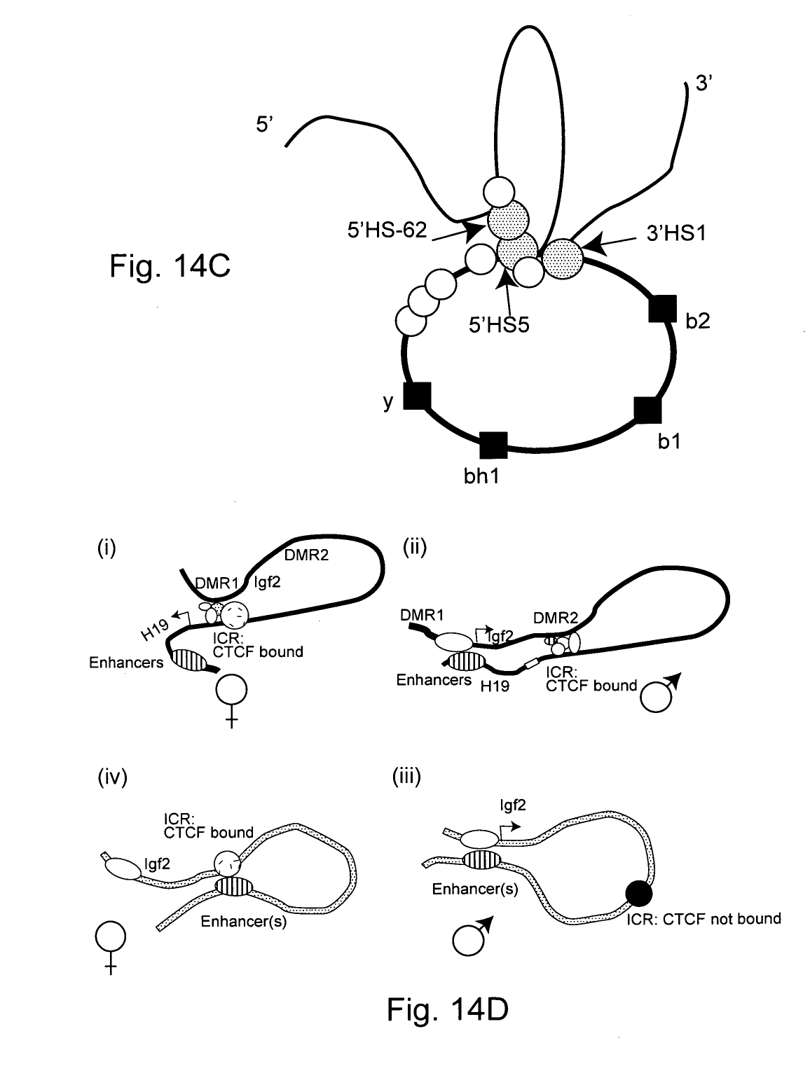

Seminal studies have demonstrated that CTCF controls imprinting at the IGF2 locus through methylation-sensitive DNA binding (Bell and Felsenfeld (2000) “Methylation of a CTCF-dependent boundary controls imprinted expression of the Igf2 gene.” Nature 405: 482-485; Hark et al. (2000) “CTCF mediates methylation-sensitive enhancer-blocking activity at the H19/Igf2 locus.” Nature 405: 486-489; Holmgren et al. (2001) “CpG methylation regulates the Igf2/H19 insulator.” Curr Biol 11: 1128-1130). (A model of CTCF binding in the Igf2/H19 imprinting control region is depicted in FIG. 14.) In fact, loss of imprinting is one of the most frequent alterations in human cancers and is observed at IGF2 in colorectal tumors (Jelinic and Shaw (2007) “Loss of imprinting and cancer.” J Pathol 211: 261-268). Although histone modification precedes DNA methylation during the course of gene silencing (Bachman et al. (2003) “Histone modifications and silencing prior to DNA methylation of a tumor suppressor gene.” Cancer Cell 3: 89-95; Strunnikova et al. (2005) “Chromatin inactivation precedes de novo DNA methylation during the progressive epigenetic silencing of the RASSF1A promoter.” Mol Cell Biol 25: 3923-3933), it is possible that a single aberrant DNA methylation occurrence could destabilize CTCF interaction and initiate a cascade of events culminating in epigenetic deregulation of a locus. Silencing of the p16 gene is an early step in breast carcinogenesis (Foster et al. (1998) “Inactivation of p16 in human mammary epithelial cells by CpG island methylation.” Mol Cell Biol 18: 1793-1801), which can lead to subsequent genomic instability (McDermott et al. (2006) “p16(INK4a) prevents centrosome dysfunction and genomic instability in primary cells.” PLoS Biol 4: e51) and downstream methylation events (Reynolds et al. (2006) “Tumor suppressor p16INK4A regulates polycomb-mediated DNA hypermethylation in human mammary epithelial cells.” J Biol Chem 281: 24790-24802). Therefore, dissociation of CTCF from the p16 upstream region at initial stages of tumorigenesis could have dire consequences on cell growth and genome integrity. The exact cause of impaired CTCF binding to the p16 upstream region is currently under investigation. We observed no gross defects or obvious mutations in CTCF, which remained capable of interacting with the c-Myc promoter in all cells that we examined. It is quite likely that functionally distinct CTCF complexes associate with the p16 and c-Myc genes. Such complexes can be distinguished by differences in cofactor interactions such as CHD8, YB-1, nucleophosmin (Chemukhin et al. (2000) “Physical and functional interaction between two pluripotent proteins, the Y-box DNA/RNA-binding factor, YB-1, and the multivalent zinc finger factor, CTCF.” J Biol Chem 275: 29915-29921; Ishihara et al. (2006) “CTCF-dependent chromatin insulator is linked to epigenetic remodeling.” Mol Cell 23: 733-742; Yusufzai et al. (2004) “CTCF tethers an insulator to subnuclear sites, suggesting shared insulator mechanisms across species.” Mol Cell 13: 291-298), post-translational modifications like phosphorylation and poly(ADP-ribosyl)ation (Klenova et al. (2001) “Functional phosphorylation sites in the C-terminal region of the multivalent multifunctional transcriptional factor CTCF.” Mol Cell Biol 21: 2221-2234; Yu et al. (2004) “Poly(ADP-ribosyl)ation regulates CTCF-dependent chromatin insulation.” Nat Genet. 36: 1105-1110), and interaction specificity among subsets of its 11 zinc fingers (Ohlsson et al. (2001) “CTCF is a uniquely versatile transcription regulator linked to epigenetics and disease.” Trends Genet. 17: 520-527). Thus, the absence of CTCF binding to p16, RASSF1A and CDH1 can result from defects in any of these parameters without affecting the majority of CTCF genomic functions.

Reactivating silenced tumor suppressor genes has long been a goal of cancer therapeutics. Epigenetic approaches to reestablish normal gene expression in cancer cells by agents such as AZA alone or in combination with other modifiers (Cameron et al. (1999) “Synergy of demethylation and histone deacetylase inhibition in the re-expression of genes silenced in cancer.” Nat Genet. 21: 103-107) have been used clinically for many years but with limited success (Abele et al. (1987) “The EORTC Early Clinical Trials Cooperative Group experience with 5-aza-2′-deoxycytidine (NSC 127716) in patients with colorectal, head and neck, renal carcinomas and malignant melanomas.” Eur J Cancer Clin Oncol 23: 1921-1924; Soriano et al. (2007) “Safety and clinical activity of the combination of 5-azacytidine, valproic acid, and all-trans retinoic acid in acute myeloid leukemia and myelodysplastic syndrome.” Blood 110: 2302-2308). This can result from the inability of AZA (or AZA and HDAC inhibitors) to completely restore the normal histone code (McGarvey et al. (2006) “Silenced tumor suppressor genes reactivated by DNA demethylation do not return to a fully euchromatic chromatin state.” Cancer Res 66: 3541-3549) to reestablish long-term expression of tumor suppressors (Egger et al. (2007) “Inhibition of histone deacetylation does not block resilencing of p16 after 5-aza-2′-deoxycytidine treatment.” Cancer Res 67: 346-353).

As expected, we found that AZA treatment reactivates p16 transcription in non-expressing breast cancer cells. This was associated with an increase in trimethylated H3K4 and a decrease of trimethylated H3K9, but neither CTCF binding nor H2A.Z deposition was restored. These data may have clinical applications as AZA or AZA and HDAC inhibitors are incapable of completely restoring the normal histone code (McGarvey et al. (2006) “Silenced tumor suppressor genes reactivated by DNA demethylation do not return to a fully euchromatic chromatin state.” Cancer Res 66: 3541-3549) or post-translational modifications of CTCF or other proteins to reestablish long-term expression of tumor suppressors, thus limiting their usefulness as therapeutic agents.

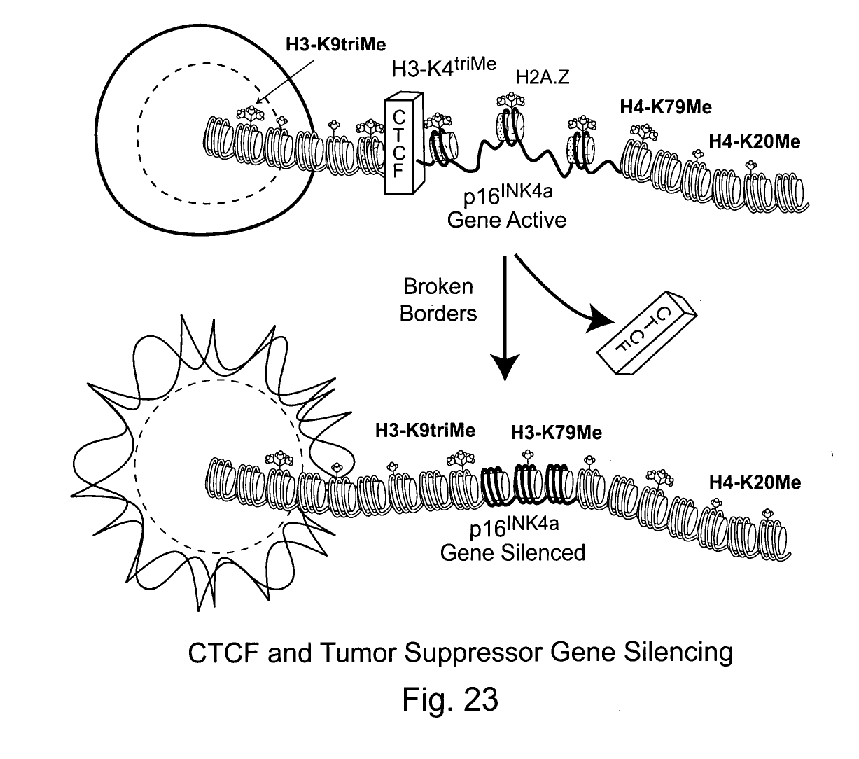

Overall, our results substantiate the critical role of CTCF in establishing and maintaining p16 and other tumor suppressor genes in higher-order chromosomal domains through appropriate boundary formation. These data raise the possibility that dissociation of CTCF from p16 during early tumorigenesis is not due to DNA methylation alone, but, rather, can result from loss of PARlated CTCF that impairs the ability of CTCF to act as a functional component of a boundary or insulator element. This can result in secondary changes in chromatin structure that are incompatible with CTCF binding to DNA. (See schematic model in FIG. 23.) When this integrity is breached by destabilized CTCF binding and loss of long-range epigenetic organization, aberrant gene silencing can ensue (FIG. 23). Thus, the ability to restore CTCF interactions at vulnerable gene loci may have important therapeutic implications. Current efforts are now focused on targeted pharmacological intervention to restore CTCF PARlation and potentially reverse silencing of p16 and other tumor suppressor genes in human cancer cells.

Additional Details Regarding Experimental Procedures

Cell Culture

U266, KMS12 and MDA-MB-435 cell lines were maintained in RPMI-1640 media supplemented with 10% FBS (Hyclone). HeLa S3, C33A, IMR90, MDA-MB-231 and T47D cells were grown in DMEM with 10% FBS. Variant mammary epithelial cells (vHMECs) were maintained in MCDB 170 with supplements as previously described (Hammond et al. (1984) “Serum-free growth of human mammary epithelial cells: rapid clonal growth in defined medium and extended serial passage with pituitary extract.” Proc Natl Acad Sci USA 81: 5435-5439). Subconfluent cells were routinely passaged and grown in the absence of antibiotics. T47D cells were treated with 5′-AZA-2′-deoxycytidine (Sigma) at a final concentration of 10 μM.

Western Blotting and RT-PCR

Nuclear extracts were separated on 10% SDS-PAGE, transferred to nitrocellulose membranes, and blotted using antibodies against CTCF, Poly(ADP-Ribose) polymers (Upstate), Actin (Sigma), Topo IIβ, PARP-1, Nucleolin, Nucleophosmin (Santa Cruz), and phosphoserine (Zymed). For RT-PCR assays, cDNA was made from 500 ng of total RNA using the Superscript II kit (Invitrogen).

### Chromatin Immunoprecipitations

ChIPs were performed according to the Upstate Biotechnology protocol with some modifications. Briefly, 2×10⁶ cells per immunoprecipitation were collected and crosslinked in full media with formaldehyde (final concentration=1%) for 10 minutes at 37° C. Crosslinking reactions were terminated with 125 mM Glycine. Cells were washed with PBS containing 1 mM PMSF and resuspended in 200 μl ChIP lysis buffer (1% SDS, 10 mM EDTA, 50 mM Tris-HCl, pH 8.1). After chilling on ice, samples were sonicated to appropriate DNA length using a Fisher 550 sonicator (15 bursts of 15 seconds duration at power 3). Next, each sample was diluted to 1.2 ml with 1.0 ml of ChIP IP buffer (0.01% SDS, 1.1% Triton X-100, 1.2 mM EDTA, 16.7 mM Tris-HCl, pH 8.1) with protease inhibitors. This chromatin solution was pre-cleared with 60 μl of a 50% protein A/G bead slurry at 4° C. Afterwards, the supernatant was collected and proteins were immunoprecipitated overnight at 4° C. by the addition of antibody. Chromatin complexes were captured with 40 μl of a 50% protein A/G slurry supplemented with 2.5 mg/ml BSA and 200 μg/ml salmon sperm DNA for 4 hours. Beads were collected and washed 6 times with 1 ml of the following wash solutions for 5 minutes each: Wash 1=0.1% SDS, 1% Triton, 2 mM EDTA, 20 mM Tris-HCl, pH 8.0, 150 mM NaCl; Wash 2=Wash 1 except with 300 mM NaCl; Wash 3=Wash 1 except with 500 mM NaCl; Wash 4=0.25 M LiCl, 1% NP-40, 1% sodium deoxycholate, 1 mM EDTA, 10 mM Tris-HCl, pH 8.0; Wash 5 and 6=Tris-EDTA, pH 8.0. Complexes were eluted from beads twice with 250 μl Elution buffer (1% SDS in 0.1 M NaHCO₃) for 15 minutes. Formaldehyde crosslinks were reversed with the addition of 20 μl 5M NaCl and incubation at 65° C. overnight. Next, proteins were digested by the addition of 10 μl 0.5M EDTA, 20 μl 1M Tris-HCl, pH 6.8 and 2 μl of 10 mg/ml Proteinase K for 2 hours at 45° C. DNA was recovered by phenol-chloroform extraction followed by ethanol precipitation using yeast tRNA as a carrier. Amplification of DNA was carried out within a linear range of all primers. Antibody sources for ChIP: bulk H3, H2A.Z, monomethylated H3K79, monomethylated H4K20 (Abcam); bimethylated H3K27, trimethylated H3K9, trimethylatedH3K4, CTCF, Poly(ADP-Ribose) polymers (Millipore); and Topo PARP-1 (Santa Cruz).

CTCF and PARP-1 Knockdown

pSHAG-MAGIC2 retroviral vectors encoding CTCF-specific or scrambled shRNAs were purchased from OpenBiosystem. Plasmid vectors were transfected in Phoenix amphotropic packaging cells using calcium phosphate/chloroqine-mediated precipitation. Supernatant containing viral particles was collected 48 hours post-transfection. Cells were infected with retrovirus and polybrene on two sequential days. 72 hours after viral exposure, successfully infected cells were selected using puromycin for a further 72 hours. Protein and mRNA were collected and ChIP experiments were performed within two passages after puromycin selection.

PARP-1 knockdown was achieved using the MISSION™ Lentiviral shRNA system from Sigma. Lentiviral particles were packaged in HEK293T cells, with virus collected 24 hours post-transfection. Cells infected with shRNA-containing virus and polybrene were selected using puromycin at 72 hours post infection.

Inhibition of Transcription

MDA-MB-435 cells were treated with Flavopiridol (Sigma) or Actinomycin D (Sigma) at 1 μM and 2.5 μg/ml, respectively, for 24 hours. At this time cells were harvested and analyzed for gene expression and CTCF binding.

Immunofluorescence

Immunofluorescence was performed with a Zeiss Axioplan 2 microscope using software from Openlab and Improvision as previously described (Verdun et al. (2005) “Functional human telomeres are recognized as DNA damage in G2 of the cell cycle.” Mol Cell 20: 551-561) except that cells were fixed with a 90:10 mix of methanol-acetic acid on ice. CTCF antibody (Upstate) was used at a 1:200 dilution and secondary FITC-coupled anti-rabbit antibody (Jackson Laboratories) was used at a 1:300 dilution.

Co-Immunoprecipitations

2 mg of whole cell lysates were diluted in IP buffer with 0.5% Triton X-100. Protein mixes were pre-cleared for 1-2 hours with protein G Sepharose, after which the beads were removed and CTCF (Upstate) or anto-phosphotyrosine (Upstate) antibody added overnight at 4° C. to capture complexes. Complexes were recovered with protein G Sepharose, washed 4 times in IP buffer and subsequently analyzed by SDS-PAGE.

In Vitro Binding of PARP-1 and CTCF

Reactions were performed such that PARP-1 was catalytically active in presence of 1 mM β-NAD⁺. Reaction buffer contained 20 mM Tris-HCl, pH 8.0, 1 mM MgCl₂, 1 mM DTT, 50 ng salmon sperm DNA, 50 ng BSA. 250 ng of recombinant CTCF (isolated from overexpressing NIH3T3 cells) or PARP-1 (Alexis Biochemicals) protein was added where appropriate. Binding was carried out at 30° C. for 1 hour. At this time, reactions were diluted in 0.5% Triton IP buffer and CTCF was immunoprecipitated as described.

Bisulphite Sequencing

2.5 μg genomic DNA was digested with Eco RV followed by repurification. DNA was denatured at 95° C. for 15 minutes, cooled on ice and then denatured with 0.3M NaOH at 37° C. for 20 minutes. After this, hydroquinone was added to a final concentration of 1.3 mM and sodium metabisulphite was added to a final concentration of 3M. Reaction mixes were subjected to the following heating procedure: 4 times in thermal cycler at 55° C. 4 hr, 90° C. 2 min, 20° C. 10 min. Next, DNA was isolated from the reaction mix using DNA binding columns (Qiagen). Resupended DNA was treated with NaOH at a concentration of 0.3M for 20 minutesat room temperature. Sodium acetate (pH 5.4) was added to a concentration of 3M and DNA was precipitated with ethanol. Recovered DNA was resuspended in water and amplified using primers specific for bisuphite-modified DNA.

Expression of CTCF in T47D Cells

Full length CTCF cloned from IMR90 cells was inserted into an HA-tagged lentiviral packaging vector. Lentivirus was produced and delivered as described above. The parent vector was also used to infect cells as a control. Anti-HA western blots were done using the F-7 antibody from Santa Cruz.

While the foregoing invention has been described in some detail for purposes of clarity and understanding, it will be clear to one skilled in the art from a reading of this disclosure that various changes in form and detail can be made without departing from the true scope of the invention. For example, all the techniques and methods described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of identifying a compound that binds to or modulates an activity of a CTCF polypeptide or CTCF polypeptide complex, the method comprising:
(a.) contacting a biological or biochemical sample comprising the polypeptide or complex with a test compound; and,
(b.) detecting binding of the test compound to the polypeptide or complex, or modulation of the activity of the polypeptide or complex by the test compound, thereby identifying the compound that binds to or modulates the activity of the CTCF polypeptide or complex.

2. The method of claim 1, wherein the sample comprises a tumor suppressor gene and wherein the activity comprises suppression of gene silencing of the tumor suppressor gene or restoration of tumor suppressor gene expression.

3. The method of claim 1, wherein the activity comprises:
(a) induction or loss of tumorigenesis in a cell present in the biological or biochemical sample;
(b) binding of the polypeptide or the complex to a histone, a post-translationally modified histone, a chromatin, or a chromatin boundary in the biological or biochemical sample;
(c) chromatin boundary stabilization, insulation, or formation, or suppression of a loss of a chromosome boundary during gene silencing in the biological or biochemical sample;
(d) binding of the polypeptide or complex to a chromatin boundary within or proximal to an INK4/ARF gene locus, a p16^{INK4a} gene, a RASSF1A gene, a CDH1 gene or a C-Myc gene present in the biological or biochemical sample;
(e) activation of a p16^{INK4a} gene, a RASSF1A gene, a CDH1 gene or a C-Myc gene present in the biological or biochemical sample; or,
(f) stabilization of tumor suppressor gene reactivation for a tumor suppressor gene present in the biological or biochemical sample.

4-8. (canceled)

9. The method of claim 1, wherein the activity comprises one or more of:
an increase or decrease in aberrant methylation in or proximal to a promoter or gene of interest;
an increase or decrease in H2A.Z binding proximal to or within a promoter or gene of interest;
an increase or decrease in trimethylation of H3K4 proximal to or within a promoter or gene of interest;
an increase or decrease in monomethylation of H4K20 proximal to or within a promoter or gene of interest;
an increase or decrease in dimethylation of H3K27 proximal to or within a promoter or gene of interest; or
an increase or decrease in trimethylation of H3K9 proximal to or within a promoter or gene of interest.

10. The method of claim 1, wherein the activity comprises formation of an active CTCF polypeptide complex in the biological or biochemical sample.

11. The method of claim 10, wherein the active CTCF polypeptide complex is a gene specific complex.

12. The method of claim 11, wherein the gene is p16 and wherein the active CTCF polypeptide complex comprises Topoisomerase

13. The method of claim 10, wherein the active complex comprises:
(a) CHD8, Topoisomerase IIα, Topoisomerase IIβ, Nucleophosmin, Poly(ADP-ribose) polymerase (PARP-1), Importin alpha3/alpha1, Lamin A/C, YB-1, Nucleolin, a TFII-i, or YY1;
(b) a DNA repair enzyme, RAD50, MRE11, XRCC6/KU80, or a SWI/SNF chromatin remodeling enzyme; or,
(c) H2A.Z.

14-15. (canceled)

16. The method of claim 10, wherein the active complex comprises one or more post-translational modification.

17. The method of claim 1, wherein the biological or biochemical sample comprises a cancer cell, a multiple myeloma cell, a U266 cell, a KMS12 cell, a breast cancer cell, a T4D7 cell, a primary breast epithelial cancer cell, a vHMEC cell, a cervical cancer cell, a normal human mammary epithelial cell (HMEC), a HeLa cell, a non-transformed fibroblast cell, an MDA-MB-435 cell, an IMR90 cell, a primary cancer cell from a patient, or a cell derived through culture from a primary cancer cell from a patient.

18. The method of claim 1, wherein the method comprises screening a plurality of test compounds by performing steps (a) and (b) for each of the plurality of test compounds.

19. The method of claim 18, wherein the plurality of compounds is prescreened for one or more of: bioavailability, toxicity, and transport to the nucleus.

20. The method of claim 18, wherein the test compound is selected from the group consisting of: a kinase inhibitor, a phosphatase inhibitor, a post-translational modification reagent, a nucleoside analogue, a nucleotide analogue, a methylation reagent, a hypomethylating nucleoside analogue, an HDAC inhibitor, a polypeptide, a naturally occurring compound, and a small organic molecule.

21. The method of claim 18, wherein the test compound is a member of a combinatorial compound library.

22. The method of claim 21, wherein the combinatorial compound library is selected to comprise a majority of members that conform to Lipinsky's rule of 5, requiring that each member of the majority comprise not more than 5 hydrogen bond donors, not more than 10 hydrogen bond acceptors, a molecular weight under 500 g/mol and a partition coefficient log P less than 5.

23. The method of claim 21, wherein the combinatorial compound library is based upon at least one pharmacophore scaffold.

24. The method of claim 21, wherein the combinatorial compound library is based upon up to about 45 different pharmacophore scaffolds, where each scaffold is represented in the library by a plurality of members, and the overall library comprises at least about 4,000 unique compounds.

25. The method of claim 24, wherein each scaffold is represented, on average, by at least about 96 members.

26. The method of claim 1, wherein the compound induces or potentiates the activity of the CTCF polypeptide or complex.

27. The method of claim 27, wherein the compound induces or potentiates the activity of the CTCF polypeptide or complex by promoting poly(ADP-ribosyl)ation of CTCF or by promoting poly(ADP-ribosylation) of a CTCF-associated cofactor.

28. (canceled)

29. The method of claim 1, wherein the compound inhibits the activity of the CTCF polypeptide or complex.

30. The method of claim 29, wherein the compound inhibits the activity of the CTCF polypeptide or CTCF polypeptide complex by preventing poly(ADP-ribosyl)ation of CTCF or by preventing poly(ADP-ribosylation) of a CTCF-associated cofactor.

31. (canceled)

32. A method of monitoring a cancer or age-related disease state of a cell, the method comprising detecting destabilization of a chromatin boundary proximal to a tumor suppressor gene of the cell, wherein destabilization of the chromatin boundary correlates with genomic instability or a tumorigenesis process in the cell.

33-40. (canceled)

41. A method of selecting a treatment or determining a prognosis for a cancer- or age-related disease, the method comprising:
measuring CTCF protein or CTCF complex binding within or proximal to a gene in a patient, wherein CTCF protein or complex binding within or proximal to gene is correlated with disease progression, or treatment selection; and,
providing a patient prognosis based upon said CTCF protein or complex binding, or
selecting a treatment course based upon said CTCF protein or complex binding.

42-50. (canceled)

51. A recombinant cell, comprising:
a recombinant gene comprising a gene encoding CTCF under the control of a heterologous promoter; and,
a recombinant gene comprising a tumor suppressor promoter operably linked to a reporter.

52-61. (canceled)


SEQUENCE LISTING (38 sequences)

SEQ ID NO: 1 - DNA, 20 bp, Artificial Sequence
  Oligonucleotide
tattcctccattgcctttgc

SEQ ID NO: 2 - DNA, 20 bp, Artificial Sequence
  Oligonucleotide
tggggtgatgcattctgata

SEQ ID NO: 3 - DNA, 20 bp, Artificial Sequence
  Oligonucleotide
actctcccacccccattaag

SEQ ID NO: 4 - DNA, 20 bp, Artificial Sequence
  Oligonucleotide
atgctgccatacccagctaa

SEQ ID NO: 5 - DNA, 20 bp, Artificial Sequence
  Oligonucleotide
gagaggtaccccgaggaaaa

SEQ ID NO: 6 - DNA, 22 bp, Artificial Sequence
  Oligonucleotide
ccctggttgacttaaaccttgt

SEQ ID NO: 7 - DNA, 20 bp, Artificial Sequence
  Oligonucleotide
tggtctttggatcactgtgc

SEQ ID NO: 8 - DNA, 20 bp, Artificial Sequence
  Oligonucleotide
taatacggacgggggagaat

SEQ ID NO: 9 - DNA, 19 bp, Artificial Sequence
  Oligonucleotide
gggctctcacaactaggaa

SEQ ID NO: 10 - DNA, 21 bp, Artificial Sequence
  Oligonucleotide
cggaggaggtgctattaactc

SEQ ID NO: 11 - DNA, 20 bp, Artificial Sequence
  Oligonucleotide
tcacagtgctctctgcctgt

SEQ ID NO: 12 - DNA, 20 bp, Artificial Sequence
  Oligonucleotide
acacaagccccaggtgtcta

SEQ ID NO: 13 - DNA, 20 bp, Artificial Sequence
  Oligonucleotide
gccattaccggttctccata

SEQ ID NO: 14 - DNA, 20 bp, Artificial Sequence
  Oligonucleotide
caggcggttccttaaaacaa

SEQ ID NO: 15 - DNA, 20 bp, Artificial Sequence
  Oligonucleotide
gccattaccggttctccata

SEQ ID NO: 16 - DNA, 20 bp, Artificial Sequence
  Oligonucleotide
caggcggttccttaaaacaa

SEQ ID NO: 17 - DNA, 20 bp, Artificial Sequence
  Oligonucleotide
ccgaagtcagttccttgtgg

SEQ ID NO: 18 - DNA, 19 bp, Artificial Sequence
  Oligonucleotide
ctgtgaacgcagcacacac

SEQ ID NO: 19 - DNA, 20 bp, Artificial Sequence
  Oligonucleotide
tttgcttctgggcagaactt

SEQ ID NO: 20 - DNA, 20 bp, Artificial Sequence
  Oligonucleotide
ggggctgcctatgtagtgaa

SEQ ID NO: 21 - DNA, 20 bp, Artificial Sequence
  Oligonucleotide
tatagcctgggcaagtcctg

SEQ ID NO: 22 - DNA, 20 bp, Artificial Sequence
  Oligonucleotide
gtacagggcgatccacactt

SEQ ID NO: 23 - DNA, 20 bp, Artificial Sequence
  Oligonucleotide
attcaggagctgctggtcac

SEQ ID NO: 24 - DNA, 20 bp, Artificial Sequence
  Oligonucleotide
tgtccttcagggagaacagg

SEQ ID NO: 25 - DNA, 20 bp, Artificial Sequence
  Oligonucleotide
gcctagccccaagtagactg

SEQ ID NO: 26 - DNA, 20 bp, Artificial Sequence
  Oligonucleotide
tgggcaggtaaaaggaagtg

SEQ ID NO: 27 - DNA, 20 bp, Artificial Sequence
  Oligonucleotide
tagagggtcaccgcgtctat

SEQ ID NO: 28 - DNA, 20 bp, Artificial Sequence
  Oligonucleotide
tcacaggtgctttgcagttc

SEQ ID NO: 29 - DNA, 20 bp, Artificial Sequence
  Oligonucleotide
tcaggagcctctaggagcag

SEQ ID NO: 30 - DNA, 20 bp, Artificial Sequence
  Oligonucleotide
tcaggcagtcttgtcccttt

SEQ ID NO: 31 - DNA, 20 bp, Artificial Sequence
  Oligonucleotide
tcaggagcctctaggagcag

SEQ ID NO: 32 - DNA, 20 bp, Artificial Sequence
  Oligonucleotide
tcaggcagtcttgtcccttt

SEQ ID NO: 33 - DNA, 20 bp, Artificial Sequence
  Oligonucleotide
acaattttgtgcgtccatca

SEQ ID NO: 34 - DNA, 20 bp, Artificial Sequence
  Oligonucleotide
accaatggccagctgttaag

SEQ ID NO: 35 - DNA, 20 bp, Artificial Sequence
  Oligonucleotide
actctccctccctgcctaac

SEQ ID NO: 36 - DNA, 20 bp, Artificial Sequence
  Oligonucleotide
caatctaccctgcagccatt

SEQ ID NO: 37 - DNA, 20 bp, Artificial Sequence
  Oligonucleotide
aaggcgcacagaggaattta

SEQ ID NO: 38 - DNA, 20 bp, Artificial Sequence
  Oligonucleotide
ttaaaatgagcaggggagga